(19) United States
(12) Patent Application Publication
(10) Pub. No.: US 2023/0405109 A1
(43) Pub. Date: Dec. 21, 2023

(54) NUCLEIC ACIDS, PROTEINS, AND VACCINES OF SARS-COV-2

(71) Applicant: City of Hope, Duarte, CA (US)

(72) Inventors: Larry W. Kwak, Duarte, CA (US); Soungchul Cha, Duarte, CA (US); Szymon Szymura, Duarte, CA (US)

(21) Appl. No.: 18/033,980

(22) PCT Filed: Nov. 11, 2021
(86) PCT No.: PCT/US2021/058929

§ 371 Date: Apr. 26, 2023

Related U.S. Application Data
(60) Provisional application No. 63/112,591, filed on Nov. 11, 2020.

(51) Int. Cl. A61K 39/215; A61P 37/04

(52) U.S. Cl. CPC A61K 39/215; A61P 37/04; A61K 2039/53

(57) ABSTRACT

Provided herein are, inter alia, SARS-CoV-2 spike (S) proteins; nucleic acids and plasmids encoding the proteins; vaccines and pharmaceutical compositions comprising the proteins, nucleic acids, or plasmids; methods for treating or preventing COVID-19; and methods for increasing immunity or providing acquired immunity to SARS-CoV in a subject.

### CROSS-REFERENCE TO RELATED APPLICATIONS

This application claims priority to U.S. Application No. 63/112,591 filed Nov. 11, 2020, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440776001WO.txt, created 2121, x bytes, machine format IBM-PC, MS Windows operating system, is hereby incorporated by reference.

### BACKGROUND

Due to the epidemiological progression and rapid spread of the COVID-19 pandemic, caused by the SARS-CoV-2 virus, America has become one of the major epicenters of the crisis.

Effective vaccines are needed to manage the spread of COVID-19 and to prevent future outbreaks. Provided herein are solutions to these and other problems in the art.

### BRIEF SUMMARY

Provided herein are proteins having at least 85% sequence identity to SEQ ID NO:4, SEQ ID NO:6, SEQ ID NO:8, SEQ ID NO:10, SEQ ID NO:12, SEQ ID NO:14, SEQ ID NO:16, or SEQ ID NO:2. Provided herein are nucleic acids encoding proteins having at least 85% sequence identity to SEQ ID NO:4, SEQ ID NO:6, SEQ ID NO:8, SEQ ID NO:10, SEQ ID NO:12, SEQ ID NO:14, SEQ ID NO:16, or SEQ ID NO:2. Provided herein are plasmids containing nucleic acids encoding proteins having at least 85% sequence identity to SEQ ID NO:4, SEQ ID NO:6, SEQ ID NO:8, SEQ ID NO:10, SEQ ID NO:12, SEQ ID NO:14, SEQ ID NO:16, or SEQ ID NO:2. In embodiments, the protein does not comprise the HA-tag having SEQ ID NO:31. Provided herein are nucleic acids encoding these proteins, and plasmids containing the nucleic acids.

Provided herein are nucleic acids having at least 85% sequence identity to SEQ ID NO:3, SEQ ID NO:5, SEQ ID NO:7, SEQ ID NO:9, SEQ ID NO:11, SEQ ID NO:13, SEQ ID NO:15, or SEQ ID NO:1. Provided herein are plasmids containing nucleic acids having at least 85% sequence identity to SEQ ID NO:3, SEQ ID NO:5, SEQ ID NO:7, SEQ ID NO:9, SEQ ID NO:11, SEQ ID NO:13, SEQ ID NO:15, or SEQ ID NO:1. In embodiments, the nucleic acids do not comprise the HA-tag having SEQ ID NO:30.

Provided herein are proteins comprising an amino acid sequence of formula (I): R¹-L¹-R²-L²-R³-R⁴-R⁵, wherein R¹ is an amino acid sequence having at least 85% sequence identity to SEQ ID NO:46; L¹ is SEQ ID NO:20, absent, —S(G₄S)ₓ—, -(G₄S)ₓ—, —S(G₃S)ₓ—, or -(G₃S)ₓ—; wherein x is an integer from 1 to 10; R² is an amino acid sequence having at least 85% sequence identity to SEQ ID NO:47; L² is SEQ ID NO:21, absent, —S(G₄S)ₓ—, -(G₄S)ₓ—, —S(G₃S)ₓ—, or -(G₃S)ₓ—; wherein x is an integer from 1 to 10; R³ is an amino acid sequence having at least 85% sequence identity to SEQ ID NO:48; R⁴ is absent, an amino acid sequence having at least 85% sequence identity to SEQ ID NO:41, or a truncated transmembrane domain; and R³ is absent or a truncated cytoplasmic domain; provided that R⁵ is absent when R⁴ is absent or a truncated transmembrane domain. In embodiments, R⁴ is an amino acid sequence having at least 85% sequence identity to SEQ ID NO:41 and R⁵ is absent. In embodiments, R⁴ is absent and R⁵ is absent. In embodiments, the protein further comprises a human MIP3α protein has the amino acid sequence of SEQ ID NO:29. Provided herein are nucleic acids encoding the protein of formula (I), and plasmids comprising the nucleic acid encoding the protein of formula (I).

Provided herein are SARS-CoV-2 spike (S) proteins comprising a truncated cytoplasmic domain; SARS-CoV-2 spike (S) protein comprising a truncated transmembrane domain; wherein the protein does not comprise a cytoplasmic domain; SARS-CoV-2 spike (S) proteins that do not comprise a transmembrane domain or a cytoplasmic domain; and SARS-CoV-2 spike (S) proteins wherein the S1/S2 protease cleavage site and/or S2′ protease cleavage site are absent or replaced with a peptide linker. Provided herein are nucleic acids encoding these proteins, and plasmids containing the nucleic acids.

Provided herein are methods of increasing immunity to a SARS coronavirus or providing acquired immunity to SARS coronavirus to a human in need thereof by administering an effective amount of a protein, nucleic acid, or plasmid, as provided herein; a pharmaceutical composition comprising a protein, nucleic acid, or plasmid, as provided herein; or a vaccine comprising a protein, nucleic acid, or plasmid, as provided herein. In embodiments, the SARS coronavirus is SARS-CoV-2.

Provided herein are methods of preventing COVID-19 in a human in need thereof by administering an effective amount of a protein, nucleic acid, or plasmid, as provided herein; a pharmaceutical composition comprising a protein, nucleic acid, or plasmid, as provided herein; or a vaccine comprising a protein, nucleic acid, or plasmid, as provided herein.

### BRIEF DESCRIPTION OF THE DRAWINGS

FIGS. 1A-1B present data showing immunization and long term memory immune responses in macaques administered with a plasmid DNA encoding MCP3-gp120 fusion protein. FIG. 1A presents graphs showing the immunization of rhesus macaques with plasmid DNA encoding MCP3-gp120 fusion primed viral-reactive T-cell immunity, which was boosted by HIV-1 envelope peptide-cocktail vaccine. Viral-reactive T-cell immunity in peripheral blood mononuclear cells collected after DNA vaccination is shown by the cell proliferation (panel (a)) and IFN-γ producing cells (panel (b)) in response to in vitro stimulation with cell-free, heat-inactivated SHIV89.6P. Antigen-specific T-cell immunity was boosted by the peptide cocktail vaccine, especially in monkey #J160 showing significant elevation of IFN-γ producing cells in response to heat-inactivated SHIV89.6P (panel (b)). The immunogenicity of the peptide vaccine was confirmed by analysis of peptide-specific T-cell immunity (panel (c)). IL-6 was predominantly detected in both macaques after DNA vaccination at Week 12 (panel (d)). Virus-reactive IL-6 secretion was more apparent in monkey #J160, particularly during the period of peptide-cocktail boost. FIG. 1B presents Fluorescence Assisted Cell Sorting (FACS) plots showing mucosal long term memory T-cell immune responses to the immunization strategy presented in FIG. 1A, as shown by production of IFN-γ by CD3+CD4+ or CD3+CD8+memory T cells isolated from colon in the vaccinated macaques one year after final peptide-cocktail boost. Lamina propria lymphocytes (LPL) from colon biopsy samples were stimulated with peptide-mix or mitogens for 6 h. Both untreated (control) and stimulated cells were stained for surface markers, followed by fixation, permeabilization, and intracellular staining of IFN-γ. Live cells were identified by gating on Aqua-negative cells. The cells gated on CD3+CD4+ and CD3+CD8+ were further separated as memory population according to the expression of CD95. The percentage values indicate the population of IFN-γ producing CD3+CD95+CD4+ or CD3+CD95+CD8+ lymphocytes.

FIGS. 2A-2D present results from phase 1 clinical trial of chemokine-antigen fusion DNA vaccines, showing favorable immune perturbation of the LPL tumor microenvironment (TMC) post-vaccination. FIG. 2A shows Uniform Manifold Approximation and Projection (UMAP) representations of pre- and post-vaccine TMCs following graph-based clustering for representative patients LPL-005, 007, and 008 using R based Seurat analysis pipelines. FIG. 2B presents graphs showing the proportions of pre- and post-vaccine subpopulations of interest in the TMCs for in sub-populations of interest in each patient (Pt 005 [blue], Pt 007 [green], and Pt 008 [red]). FIG. 2C shows bar graphs presenting the percentage of normal B cells and LPL tumor clonotypes in pre- and post-vaccine TMCs. The percentage of tumor LPL B cells and normal B cells is shown in pre- and post-vaccine TMCs. P values (statistical difference pre/post) were calculated using 2×5 Fisher's exact test. White: normal healthy B cells percentage, black: LPL VH/VL tumor cells percentage, grey: LPL tumor cells that only express VL chain percentage, and red: new tumor VH/VL clonotype percentage. Additional new LPL tumor clonotypes that expressed only the VH (dark orange) and VL (light orange) chains were also observed in Patient 007. FIG. 2D presents heatmaps showing changes in T-cell frequency after vaccination. The top 20 T-cell clonotypes were first ranked from the post-vaccination T-cell repertoire. Their matching, identical clonotype was then found in the pre-vaccine T-cell repertoire and results graphed in a heatmap depicting T-cell clonotype percentages. The left vertical legend identifies the “Clone identity number” for the top 20 clonotypes, and the right vertical legend identifies the “% of clonotype T cells from clonal repertoire.” For accurate comparison between pre- and post-vaccination T-cell repertoires, p-values were calculated using the paired two-tailed Student's test.

FIGS. 3A-3B present schemas of plasmid DNA constructs comprising the chemokine-fused SARS-CoV-2 spike (S) protein. FIG. 3A is a schema of the SARS-CoV-2 S protein structure diagram including functional domains (RBD), receptor binding domain; S1/S2, S1/S2 protease cleavage site; S2′, S2′ protease cleavage site; TM, transmembrane domain; CT, cytoplasmic tail. Arrows denote protease cleavage sites. Amino acid sequences around the two protease recognition sites for wild type SARS-CoV-2-S protein are indicated in red, arrow heads indicate the cleavage site. FIG. 3B presents schemas of all constructs based on a same mammalian expression vector, regulated with the CMV early gene promoter and enhancer and the SV40 transcription termination region (polyA). The genes contain optimized Kozak 5′-untranslated regions. The SARS-CoV-2 S protein gene was cloned in frame with the murine IP-10 (SL) signal sequence with (pSARS-CoV-2-FL) or without (pSARS-CoV-2-ΔT) the TM-CT. Murine MIP-3α gene sequences were fused in-frame with DNA encoding either SARS-CoV-2 S protein (pMIP3α-SARS-CoV-2-FL), (pMIP3α-SARS-CoV-2-S) or modified SARS-CoV-2 S protein gene (pMIP3α-SARS-CoV-SL). To enable detection, HA tags were fused to the COO end of constructs (HA). Spacer fragment (SP) between MIP3α and the spike protein enables proper protein folding.

FIGS. 4A-4D present data showing that the sera from mice immunized with chemokine constructs fused with HIV gp120, particularly with gp140, elicited antibodies with a broader neutralizing activity. FIG. 4A is a graph showing that Env-protein (HIV-1, isolate 89.6)—specific serum antibody responses in pooled sera from 5 mice immunized was 5 times with DNA plasmids expressing the following proteins: secretable gp120 alone (pgp120), gp120 fused with human MIP-3 (pMCP3gp120), human MDC (pMDCgp140-14). The data shown is representative of 3 independent experiments. FIG. 4B is a graph showing pooled sera (at various dilutions, shown) from mice immunized with gp120 fused with MDC or MCP3 (pMCP3gp120 and pMDCgp120, respectively) compared with the pooled sera from mice immunized with gp140-14 fused with MDCs (pMDCgp140-14) for their ability to inhibit entry of pseudotype HIV virus expressing viral envelope proteins (Envs); the expressed Envs were HIV-1 gp160 proteins from three different HIV-1 strains (89.6, JR-FL, and NL43 strains). FIG. 4C is a graph showing pooled sera (at various dilutions, shown) from mice immunized with gp120 fused with MDC or MCP3 (pMCP3gp120 and pMDCgp120, respectively) compared with the pooled sera from mice immunized with gp140-14 fused with MDCs (pMDCgp140-14) for their ability to inhibit entry of pseudotype HIV virus expressing Envs from various isolates, such as NL4-3 for X4. FIG. 4C is a graph showing pooled sera (at various dilutions, shown) from mice immunized with gp120 fused with MDC or MCP3 (pMCP3gp120 and pMDCgp120, respectively) compared with the pooled sera from mice immunized with gp140-14 fused with MDCs (pMDCgp140-14) for their ability to inhibit entry of pseudotype HIV virus expressing Envs from various isolates, such as JRFL for R⁵. For FIGS. 4B-4D, control sera were from mice immunized with MCP3 fusion construct with EGFP (pMCP3-EGFP), or from PBS-treated mice. For FIG. 4B, P indicates comparison between pMDCgp140-14 and pMCP3gp120. For FIGS. 4C-4D, P indicates comparison with pMCP3-EGFP, P* comparison between pMDCgp140-14 and pMCP3gp120, and P** comparison between pMDCgp140-14 and pMDCgp120).

FIG. 5 is schema of a plasmid for use as clinical grade chemokine-fused SARS-CoV-2 S protein DNA vaccine. The plasmid comprises of the human MIP3 gene and leader sequence and the HA tag. The vector sequences from pUCMVC3 contain a kanamycin resistance gene, the CMV promoter, and the rabbit β-globulin polyadenylation signal.

FIG. 6 is a dot plot showing humoral response in sera from mice immunized with SARS-CoV-2 DNA vaccine candidates. In this figure, mice were immunized with four separate SARS-CoV-2 DNA vaccine candidate plasmids; plates were coated with SARS-CoV-2 spike antigen; serum was test by ELISA. Sera tested was collected two weeks after the fifth and final vaccination, after all mice were euthanized.

FIG. 7 is a schema showing the map of the pUMVC3 plasmid.

FIG. 8 present graphs showing the induction of T cell responses in Balb/c mice post-administration of cellular immunity via the DNA vaccine. Balb/c mice (n=5/group) were immunized with 100 μg DNA vaccine. T cell responses were analyzed in the animals two weeks after the fifth vaccination. T cell responses were measured by IFN-γ ELISA assays in splenocytes stimulated for 48 h with overlapping peptide 3 pools spanning the SARS-CoV-2 Spike protein. S1: spike protein 1-692aa peptide pools; S+: spike protein 689-895aa peptide pools; S: 304-338aa, 421-475aa, 492-519aa, 683-707aa, 741-770aa, 785-802aa and 885-1273aa peptide pools. The constructs have been abbreviated to: FL: pSARS-CoV-2-FL (SEQ ID NO:1); MIP3a-F: pMIP3α-SARS-CoV-2-FL (SEQ ID NO:3); ΔT: pSARS-CoV-2-ΔT (SEQ ID NO:5); MIP3-ΔT: pMIP3α-SARS-CoV-2-ΔT (SEQ ID NO:7). Two-tailed unpaired Student's t-test was utilized for statistical analysis. Bars represent the mean+SD. Due to technical issues in group FL, two mice were not included in the IFN-γ ELISA assay.

FIGS. 9A-9D show antibody responses in immunized BALB/c mice against the SARS-CoV-2 Spike protein. FIG. 9A: the experimental timeline for BALB/c mice immunized with our SARS-CoV-2 Spike “S” DNA vaccines is shown over a course of 10 weeks. Humoral and cellular immune responses were detected post-euthanasia 10 weeks after the first vaccination. FIGS. 9B-9E: antibody response against the SARS-CoV-2 Spike “S” protein in BALB/c mice immunized with pSARS-CoV-2 FL (SEQ ID NO:1)(FIG. 9B), pMIP3α-SARS-CoV-2-FL (SEQ ID NO:3)(FIG. 9C), pSARS-CoV-2-ΔT (SEQ ID NO:5)(FIG. 9D), and pMIP3α-SARS-CoV-2-ΔT (SEQ ID NO:7)(FIG. 9E) using a needle-free PharmaJet® injector. Mice were immunized with four separate SARS-CoV-2 DNA vaccine candidate plasmids; plates were coated with SARS-CoV-2 spike antigen; serially diluted serum was tested by ELISA. Sera tested was collected two weeks after the fifth and final vaccination, after all mice were euthanized.

FIG. 10 shows the total area under the curve (AUC) of Antibody Response in Immunized BALB/c mice Against the SARS-CoV-2 Spike protein. Total calculated area under the curve (AUC) of the measured antibody response (FIG. 9) against the SARS-CoV-2 S protein in BALB/c mice immunized with pSARS-CoV-2-FL (SEQ ID NO:1), pMIP3α-SARS-CoV-2-FL (SEQ ID NO:3), pSARS-CoV-2-ΔT (SEQ ID NO:5), pMIP3α-SARS-CoV-2-ΔT (SEQ ID NO:7), and PBS using a needle-free Pharmajet injector. The baseline for AUC calculations was the mean value of the antibody response levels for mice immunized with PBS as a negative control. BALB/c mice were immunized with four separate SARS-CoV-2 DNA vaccine candidate plasmids; plates were coated with SARS-CoV-2 spike antigen; serially diluted serum was tested by ELISA. Sera tested was collected two weeks after the fifth and final vaccination, after all mice were euthanized. For accurate comparisons between experimental vaccine constructs, p values were calculated using the unpaired two-tailed Student's test.

FIGS. 11A-11B show antibody response against the spike protein over the course of five vaccinations in immunized BALB/c mice. Antibody response against the SARS-COV-2 Spike “S” protein in BALB/c mice immunized with pSARS-CoV-2 FL (SEQ ID NO:1) (FIG. 11A), pMIP3α-SARS-CoV-2-FL (SEQ ID NO:3) (FIG. 11B) over the course of five subsequent vaccinations using a needle-free Pharmajet injector. The x-axis depicts the time passed in weeks after the first vaccination. ELISA plates were coated with SARS-CoV-2 spike antigen, and a 1:200 dilution of serum was tested by ELISA. Sera was collected prior to each vaccination and two weeks after the fifth and final immunization over the course of 10 weeks.

FIGS. 12A-12G show antibody responses against the SARS-CoV-2 Spike protein and the top mutational variants of the Receptor Binding Domain (RBD) proteins in K18-hACE2 mice immunized with pSARS-CoV-2 FL (SEQ ID NO:1), pMIP3α-SARS-CoV-2-FL (SEQ ID NO:3), and PBS using a needle-free Pharmajet injector. Plates were coated with SARS-CoV-2 spike antigen (Wuhan-Hu-1 Isolate)(FIG. 12A), RBD protein (Wuhan-Hu-1 Isolate) (FIG. 12B), Alpha B.1.1.7 Isolate RBD Protein (United Kingdom) (FIG. 12C), Delta B.1.617.1 Isolate RBD Protein (India) (FIG. 12D), Delta B.1.617.2 Isolate RBD Protein (India) (FIG. 12E), the Beta B.1.351 Isolate RBD protein (South Africa) (FIG. 12F), and the Gamma P.1 RBD protein (Brazil) (FIG. 12G); serially diluted serum was tested by ELISA. Sera collected from mice immunized with PBS was used as a negative control and a positive control for ELISA was SARS-CoV-2 positive serum collected from rhesus macaques (BEI Resources Manassas, VA). Sera tested was collected two weeks after the third and final vaccination, after all mice were euthanized.

FIG. 13 shows total area under the curve (AUC) of antibody response in immunized K18-hACE2 mice against the Spike protein and top mutational variants of the Receptor Binding Domain (RBD) proteins for SARS-CoV-2. Total calculated area under the curve (AUC) of the measured antibody response (FIG. 12) against the spike protein and top mutational variants of the receptor binding domain protein in K18-hACE2 mice immunized with pSARS-CoV-2 FL (SEQ ID NO:1), pMIP3α-SARS-CoV-2-FL (SEQ ID NO:3), and PBS using a needle-free Pharmajet injector. Orange AUC values are from sera collected from K18-hACE2 mice immunized with pSARS-CoV-2 FL, and red AUC values are from sera collected from K18-hACE2 mice immunized with pMIP3α-SARS-CoV-2-FL. Plates were coated with SARS-CoV-2 spike antigen, RBD protein (Wuhan-Hu-1 Isolate), Alpha B.1.1.7 Isolate RBD Protein, Delta B.1.617.1 Isolate RBD Protein, Delta B.1.617.2 Isolate RBD Protein, the Beta B.1.351 Isolate RBD protein, and the Gamma P.1 RBD protein; serially diluted serum was tested by ELISA. The baseline for AUC calculations was the mean value of the antibody response levels for mice immunized with PBS as a negative control. Sera tested was collected two weeks after the third and final vaccination, after all mice were euthanized. For accurate comparisons between antibody responses to the spike protein and RBD mutational variant proteins between mice immunized with pSARS-CoV-2 FL and pMIP3α-SARS-CoV-2-FL, p values were calculated using the unpaired two-tailed Student's test.

FIGS. 14A-14F are measured cytokine levels for IL-2 (FIG. 14A), IL-6 (FIG. 14B), IFN-γ (FIG. 14C), IL-5 (FIG. 14D), TNF-α (FIG. 14E), and IL-4 (FIG. 14F) in stimulated T cells collected from immunized K18-hACE2 mice. Cytokine levels in stimulated T cells collected from euthanized K18-hACE2 mice immunized with pSARS-CoV-2 FL (SEQ ID NO:1), pMIP3α-SARS-CoV-2-FL (SEQ ID NO:3), and PBS using a needle-free Pharmajet Injector. T cells were stimulated using three separate peptide pools together composing separate domains of the full length spike protein (S, S1, and S+, Miltenyi Biotec); a cytokine beads based assay was performed and measured using FLOW. T cells stimulated with the CMV pp65 peptide pool were used as an additional negative control as well as unstimulated T cells; PMA/ionomycin stimulated T cells were used as a positive control. The six bars on the left of each graft are cytokine levels in T cells from mice immunized with pSARS-CoV-2 FL, the six bars in the middle of each graph are from mice immunized with pMIP3α-SARS-CoV-2-FL, and the six bars on the right of each graph are from mice immunized with PBS. T cells were isolated from spleen collected from euthanized K18-hACE2 mice two weeks after the third and final vaccination, after all mice were euthanized. S peptide pool covers the predicted immunodominant domains of the SARS-CoV-2 spike glycoprotein (protein S; 304-338, 421-475, 492-519, 638-707, 741-770, 785-802, and 885-1273), S1 peptide pool covers the N-terminal S1 domain (1-692), S+ peptide pool covers a part of the C-terminal S2 domain (698-895). p values were calculated using the unpaired two-tailed Student's test. *: 0.05<P<0.01, **: 0.01<P<0.001, ***: 0.001<P<0.0001: **** P<0.0001, no*=not significant.

### DETAILED DESCRIPTION

### Definitions

As used herein, the singular forms “a”, “an” and “the” are intended to include the plural forms as well, unless the context clearly indicates otherwise.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“SARS-CoV” refers to severe acute respiratory syndrome (SARS) coronavirus (CoV). Examples of SARS-CoV include SARS-CoV-1, SARS-CoV-2, and MERS-CoV. In embodiments of the disclosure, SARS-CoV refers to SARS-CoV-1. In embodiments of the disclosure, SARS-CoV refers to SARS-CoV-2. In embodiments, the SARS-CoV-2 is the strain of coronavirus that causes COVID-19. In embodiments, the SARS-CoV-2 is a Baltimore class IV positive-sense single-stranded RNA virus that is contagious in humans. In embodiments, the SARS-CoV-2 comprises the delta variant.

“SARS-CoV-2 spike (S) protein” or “SARS-CoV-2 spike protein” or “SARS-CoV-2 S protein” or “spike protein” or “S protein” refer to the spike (S) protein of SARS-CoV-2, or variants or homologs thereof. The spike (S) protein of the SARS-CoV-2 includes any of the recombinant or naturally-occurring forms of the spike (S) protein of the SARS-CoV-2, or variants or homologs thereof, that maintain S protein activity (e.g., within at least 50%, 80%, 90%, 95%, 96%, 97%, 98%, 99% or 100% activity compared to the S protein). In embodiments, the S protein is present on the viral surface as a trimer. The S protein may include two domains, S1 and S2. In embodiments, the S1 domain mediates receptor binding and is divided into two sub-domains, with the N-terminal subdomain (NTD) often binding sialic acid and the C-terminal subdomain (also known as C-domain) binding a specific proteinaceous receptor. In embodiments, the S2 domain mediates viral-membrane fusion through the exposure of a highly conserved fusion peptide. The fusion peptide may be activated through proteolytic cleavage at a site found immediately upstream (S2′), which is common to all coronaviruses. In many (but not all) coronaviruses, additional proteolytic priming may occur at a second site located at the interface of the S1 and S2 domains (S1/S2). In embodiments, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring the S protein polypeptide (e.g., YP_009724390.1). In embodiments, the SARS-CoV-2 spike (S) protein has the amino acid sequence in SEQ ID NO:44 or NCBI Reference Sequence: YP_009724390.1, a mutational variant, a homolog, or a functional fragment thereof. In embodiments, the SARS-CoV-2 spike (S) protein has the amino acid sequence set forth as SEQ ID NO:45, which is a functional fragment of the amino acid sequence set forth as SEQ ID NO:44 without the first fifteen amino acids (SEQ ID NO:18) at the N-terminus. In embodiments, the SARS-CoV-2 spike (S) protein is a mutational variant of the amino acid sequences described herein. In embodiments, the SARS-CoV-2 spike (S) protein has one or more mutations corresponding to, or aligning with, L5, L54, S221, V367, G476, S477, V483, D614, P681, A845, T859, or P1263 in SEQ ID NO:44. In embodiments, the SARS-CoV-2 spike (S) protein comprises a D614G mutation corresponding to or aligning with D614 of the amino acid sequence set forth as SEQ ID NO:44.

The terms “MIP3α,” “MIP-3α,” “Macrophage Inflammatory Protein 3α,” “CCL20” or “C—C motif chemokine ligand 20” refer to a chemotactic cytokine (chemokine) which binds to the chemokine receptor protein CCR6 which plays a key role in the development of adaptive immunity. This receptor has been shown to be important for B-lineage maturation and antigen-driven B-cell differentiation, and is thought to regulate the migration and recruitment of dendritic cells and T cells during inflammatory and immunological responses. MIP3α is produced by mucosa and skin by activated epithelial cells and attracts Th17 (T-helper 17) cells to the site of inflammation. MIP3α includes any of the protein naturally occurring forms, variants, or homologs, that maintain the activity of MIP3α (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the MIP3α protein is any of the proteins as identified by NCBI sequence reference NP_001123518.1 (MIP3α isoform 2 precursor), homolog or functional fragment thereof. In embodiments, MIP3α has the amino acid sequence set forth as SEQ ID NO:17. The MIP3α is binding to the receptor CCR6, and is produced by mucosa and skin by activated epithelial cells and attracts Th17 cells to the site of inflammation. It is also produced by Th17 cells themselves. It further attracts activated B cells, memory T cells and immature dendritic cells and has part in migration of these cells in secondary lymphoid organs.

The term “cytoplasmic domain” refers to the cytoplasmic domain of the spike (S) protein of SARS-CoV-2. The cytoplasmic domain has the amino acid sequence set forth as SEQ ID NO:42, which corresponds to positions 1255 to 1273 in SEQ ID NO:44. In embodiments, the cytoplasmic domain can have one or more mutations.

The term “truncated cytoplasmic domain” as used herein refers to a SARS-CoV-2 spike (S) protein cytoplasmic domain that has been shortened relative to a naturally occurring S protein polypeptide (e.g., SEQ ID NO:42). In embodiments, the SARS-CoV-2 S protein having a truncated cytoplasmic domain is able to trimerize in its soluble form to a greater extent than an equivalent S protein having a full length cytoplasmic domain. The cytoplasmic domain contains 19 amino acids, such that a truncated cytoplasmic domain comprises from 1 amino acid to 18 amino acids. In a truncated cytoplasmic domain, the amino acids are sequentially removed from the C-terminus of the cytoplasmic domain (i.e., the amino acids are not randomly removed from the cytoplasmic domain). Thus, a truncated cytoplasmic domain in which 16 amino acids have been removed means that the amino acid sequence of the truncated cytoplasmic domain is KFD (with reference to SEQ ID NO:42). As an another example stated another way, if 14 amino acids are removed from the cytoplasmic domain, the 14 amino acids that have been removed are the amino acids in SEQ ID NO:19.

The term “transmembrane domain” refers to the transmembrane domain of the spike (S) protein of SARS-CoV-2. The transmembrane domain has the amino acid sequence set forth as SEQ ID NO:41, which corresponds to positions 1209 to 1254 in SEQ ID NO:4.

The term “truncated transmembrane domain” as used herein refers to a SARS-CoV-2 spike (S) protein transmembrane domain that has been shortened relative to a naturally occurring S protein polypeptide (e.g., SEQ ID NO:41). In embodiments, the transmembrane domain is only truncated when the cytoplasmic domain has been completely removed from the SARS-CoV-2 spike (S) protein. In other words, if there are amino acids present in the cytoplasmic membrane, then the transmembrane domain cannot be truncated. The transmembrane domain contains 46 amino acids, such that a truncated transmembrane domain comprises from 1 amino acid to 45 amino acids. In a truncated transmembrane domain, the amino acids are sequentially removed from the C-terminus of the transmembrane domain (i.e., the amino acids are not randomly removed from the transmembrane domain). Thus, a truncated transmembrane domain in which 43 amino acids have been removed means that the amino acid sequence of the truncated cytoplasmic domain is YIK (with reference to SEQ ID NO:41).

The terms “linker” or “peptide linker” as used herein refer to short amino acid or peptide sequences that occur between protein domains. Linkers are often composed of flexible amino acids such as glycine (G) and/or serine (S), and allow a relative freedom of movement to adjacent protein domains. Linkers can be used when it is necessary to ensure that two adjacent domains do not sterically interfere with one another.

The terms “protease” and “protease cleavage site” is used herein according to its ordinary meaning in molecular biology. The term “protease” refers to an enzyme that catalyzes proteolysis, which is the breakdown of proteins into smaller polypeptides or single amino acids.

Proteolysis can be highly promiscuous such that a wide range of protein substrates are hydrolyzed. Promiscuous proteases can bind to a single amino acid on the substrate and so only have specificity for that residue. Conversely, some proteases are highly specific and only cleave substrates with a certain sequence.

The term “protease cleavage site” refers to a sequence recognized by a protease on a given peptide or protein. In embodiments, the protease cleavage site is the S1/S2 site of the SARS-CoV-2 spike protein, which can be referred to as the “S1/S2 protease cleavage site” of said SARS-CoV-2 spike protein. In embodiments, the S1/S2 site includes four amino acids. In embodiments, the S1/S2 site includes the sequence RRAR (SEQ ID NO:20). In embodiments, the S1/S2 is found at position 682 to 685 in the SARS-CoV-2 spike protein with reference to SEQ ID NO:44. In embodiments, the protease cleavage site is the S2′ site of the SARS-CoV-2 spike protein, which can be referred to as the “S2′ protease cleavage site” of the SARS-CoV-2 spike protein. In embodiments, the S2′ site includes four amino acids. In embodiments, the S2′ site includes the sequence PSKR (SEQ ID NO:21). In embodiments, the S2′ is found at position 812 to 815 n the SARS-CoV-2 spike protein with reference to SEQ ID NO:44.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus (chimeric poxvirus) that is capable of replicating in a cell.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic. The administration of vaccines is referred to as vaccination. In embodiments, a vaccine can provide a nucleic acid, e.g. DNA that encodes antigenic molecules (e.g. peptides) to a subject. The nucleic acid that is delivered via the vaccine in the subject can be expressed into antigenic molecules and allow the subject to acquire immunity against the antigenic molecules. In the context of the vaccination against infectious disease, the vaccine composition can provide DNA encoding antigenic molecules that are associated with a certain pathogen, e.g. one or more peptides that are known to be expressed in the pathogen (e.g. pathogenic bacterium or virus).

The term “adjuvant” or “vaccine adjuvant” or “pharmaceutically acceptable adjuvant” refer to compounds used in a vaccine to enhance (e.g., increase, accelerate, prolong, and/or target) the specific immune response to the vaccine in order to enhance the subject's immune response to the vaccine. Suitable adjuvants include aluminum salts, calcium salts, iron salts, zinc salts, acylated tyrosine, acylated sugars, cationically or anionically derivatized saccharides, polyphosphazenes, biodegradable microspheres, monophosphoryl lipid A, lipid A derivatives, 3-O-deacylated MPL, quil A, saponin, QS21, tocol, Freund's incomplete adjuvant, Adjuvant 65 (Merck and Company, Inc., Rahway, NJ), AS-2 (Glaxo-Smith-Kline, Philadelphia, PA), toll like receptor agonists (e.g., CpG ODNs), bioadhesives, mucoadhesives, microparticles, liposomes, polyoxyethylene ether formulations, polyoxyethylene ester formulations, muramyl peptides, imidazoquinolone compounds (e.g., imiquamod and its homologues), and the like. Human immunomodulators suitable for use as adjuvants include cytokines such as interleukins (e.g., IL-I, IL-2, IL-4, IL-5, IL-6, IL-7, IL-12, etc), macrophage colony stimulating factor, tumor necrosis factor, granulocyte, macrophage colony stimulating factor, and the like. In aspects, the adjuvant comprises a toll-like receptor agonist. In aspects, the adjuvant comprises an aluminum salt. In aspects, the adjuvant comprises a toll-like receptor agonist and an aluminum salt. In aspects, the adjuvant comprises an aluminum salt and monophosphoryl lipid A. In aspects, the adjuvant comprises squalene. In aspects, the adjuvant comprises monophosphoryl lipid A and QS-21. In aspects, the adjuvant comprises a toll-like receptor agonist, an aluminum salt, monophosphoryl lipid A, or a combination of two or more thereof.

In aspects, the adjuvant comprises a surfactant (e.g., hexadecylamine, octadecylamine, lysolecithin, dimethyldioctadecylammonium bromide, N,N-dioctadecyl-N′,N-bis(2-hydroxy-ethylpropane diamine), methoxyhexadecyl glycerol, pluronic polyols), polyanions (e.g., pyran, dextran sulfate, poly IC, polyacrylic acid, Carbopol), peptides (e.g., muramyl dipeptide, aimethylglycine), tuftsin, oil emulsions, B peptide subunits of E. coli, or a combination of two or more thereof. In aspects, the adjuvant comprises a surfactant.

The vaccines and compositions may be lyophilized or in aqueous form, i.e., solutions or suspensions. Liquid formulations allow the compositions to be administered direct from their packaged form, without the need for reconstitution in an aqueous medium, and are thus ideal for injection. Compositions may be presented in vials, or they may be presented in ready filled syringes or needle-free injectors. The syringes may be supplied with or without needles. A syringe will include a single dose of the composition, whereas a vial may include a single dose or multiple doses (e.g., 2, 3, or 4 doses). In aspects, the dose is for a human and may be administered by injection.

Liquid vaccines are also suitable for reconstituting other vaccines from a lyophilized form. Where a vaccine is to be used for such extemporaneous reconstitution, the disclosure provides a kit, which may comprise two vials, or may comprise one ready-filled syringe and one vial, with the contents of the syringe being used to reconstitute the contents of the vial prior to injection. Vaccines may be packaged in unit dose form or in multiple dose form (e.g. 2, 3, or 4 doses). For multiple dose forms, vials can be pre-filled syringes. Effective dosage volumes can be routinely established, but a typical human dose of the composition has an injection volume of 0.1 mL to 1 mL.

In embodiments, vaccines have a pH of between 5.5 and 8.5, and may be buffered at this pH. Stable pH may be maintained by the use of a buffer, such as a phosphate buffer or a histidine buffer. The composition should be sterile and/or pyrogen free. The compositions and vaccines may be isotonic. Vaccines may include an antimicrobial, particularly when packaged in a multiple dose format. Other antimicrobials may be used, such as 2-phenoxyethanol or parabens (methyl, ethyl, propyl parabens). Preservative may be added exogenously and/or may be a component of the bulk haptens or hapten conjugates which are mixed to form the composition (e.g. present as a preservative in pertussis antigens). Vaccines may comprise a detergent, e.g., a Tween (polysorbate), such as Tween 80. Detergents are generally present at low levels, e.g. less than 0.1%. Vaccines may include sodium salts (e.g. sodium chloride) for tonicity.

As used herein, the term “vector” refers to a nucleic acid molecule capable of transporting another nucleic acid to which it has been linked. One type of vector is a “plasmid,” which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In embodiments, “plasmid” and “vector” are used interchangeably as the plasmid is the most commonly used form of vector.

However, the disclosure is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule (e.g., mRNA, DNA, RNP) and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” or “sequence identity” in the context of two or more nucleic acids or polypeptide sequences refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the intemucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference, homolog or functional fragment thereof.

The terms “downstream” and “upstream” are used according to its plain ordinary meaning and refers to the relative positions of genetic code in DNA or RNA. Each strand of DNA or RNA has a 5′ end and a 3′ end, in relation to the carbon position on the deoxyribose (or ribose) ring of the nucleic acid being considered (respectively DNA or RNA). By convention, upstream and downstream relate to the 5′ to 3′ direction respectively in which RNA transcription takes place. Upstream is toward the 5′ end of the RNA molecule and downstream is toward the 3′ end. When considering double-stranded DNA, upstream is toward the 5′ end of the coding strand for the gene in question and downstream is toward the 3′ end. Due to the anti-parallel nature of DNA, this means the 3′ end of the template strand is upstream of the gene and the 5′ end is downstream.

The term “promoter” is used according to its plain ordinary meaning within molecular biology and refers to a sequence of DNA to which proteins bind that initiate transcription of a single RNA from the DNA downstream of it. Promoters are located near the transcription start sites of genes, upstream on the DNA (towards the 5′ region of the sense strand).

The terms “polyadenylation” and “polyadenylation signal” are used according to ordinary meaning in molecular biology. “Polyadenylation” refers to the addition of a polyadenylic acid (poly(A)) tail to a messenger RNA. The poly(A) tail consists of multiple adenosine monophosphates; in other words, it is a stretch of RNA that has only adenine bases. In eukaryotes, polyadenylation is part of the process that produces mature messenger RNA (mRNA) for translation. The term “polyadenylation signal” refers to a sequence motif recognized by the RNA cleavage complex which triggers the cleavage of a portion of the 3′ end of a newly produced RNA, prior to the polyadenylation of the RNA. In embodiments, the polyadenylation signal is a 1-globulin polyadenylation signal. In embodiments, the polyadenylation signal is a rabbit 1-globulin polyadenylation signal.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “effective dose” or “effective dosage” is defined as an amount sufficient to achieve or at least partially achieve the desired effect.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “immune response” used herein encompasses, but is not limited to, an “adaptive immune response”, also known as an “acquired immune response” or “acquired immunity” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

An “inhibitor” refers to a compound (e.g. compounds described herein) that reduces activity when compared to a control, such as absence of the compound or a compound with known inactivity.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “patient” or “subject” includes human and other mammalian subjects that receive either prophylactic or therapeutic treatment. In embodiments, the subject is human.

The term “prophylactic treatment” or “prevention” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual upon subsequent exposure to an amount of infectious viral agent that would otherwise, i.e. in the absence of such a prophylactic treatment, be sufficient to cause a viral infection.

The term “therapeutically effective dose” is defined as an amount sufficient to cure or at least partially arrest the disease, e.g. COVID-19 and its complications in a patient already suffering from the disease.

“Treatment” or “treating” refers to the administration of a therapeutic agent or combination of therapeutic agents to a patient, or application or administration of the active agent to a patient, who has a virus infection, e.g. SARS-CoV, with the purpose to cure, heal, alleviate, relieve, alter, remedy, ameliorate, improve or affect the infection, or symptoms thereof. The term “treatment” or “treating” is also used herein in the context of administering agents prophylactically. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and. This applies regardless of the breadth of the range.

SARS-CoV-2 Spike (S) Protein

Provided herein is a SARS-CoV-2 spike (S) protein comprising a truncated cytoplasmic domain. In embodiments, the truncated cytoplasmic domain comprises a deletion of one, more than one, or all amino acids from the C-terminal end of a naturally-occurring spike (S) protein cytoplasmic domain. In embodiments, the truncated cytoplasmic domain comprises a deletion of at least 1 amino acid. In embodiments, the truncated cytoplasmic domain comprises a deletion of at least 2 amino acid. In embodiments, the truncated cytoplasmic domain comprises a deletion of at least 3 amino acids. In embodiments, the truncated cytoplasmic domain comprises a deletion of at least 4 amino acids. In embodiments, the truncated cytoplasmic domain comprises a deletion of at least 5 amino acids. In embodiments, the truncated cytoplasmic domain comprises a deletion of at least 6 amino acids. In embodiments, the truncated cytoplasmic domain comprises a deletion of at least 7 amino acids. In embodiments, the truncated cytoplasmic domain comprises a deletion of at least 8 amino acids. In embodiments, the truncated cytoplasmic domain comprises a deletion of at least 9 amino acids. In embodiments, the truncated cytoplasmic domain comprises a deletion of at least 10 amino acids. In embodiments, the truncated cytoplasmic domain comprises a deletion of at least 11 amino acids. In embodiments, the truncated cytoplasmic domain comprises a deletion of at least 12 amino acids. In embodiments, the truncated cytoplasmic domain comprises a deletion of at least 13 amino acids. In embodiments, the truncated cytoplasmic domain comprises a deletion of at least 14 amino acids. In embodiments, the truncated cytoplasmic domain comprises a deletion of at least 15 amino acids. In embodiments, the truncated cytoplasmic domain comprises a deletion of at least 16 amino acids. In embodiments, the truncated cytoplasmic domain comprises a deletion of at least 17 amino acids. In embodiments, the truncated cytoplasmic domain comprises a deletion of at least 18 amino acids. In embodiments, the truncated cytoplasmic domain comprises a deletion of at least 19 amino acids. In embodiments, SARS-CoV-2 spike (S) protein does not contain a cytoplasmic domain.

In embodiments, the truncated cytoplasmic domain of the SARS-CoV-2 spike (S) protein has the amino acid sequence corresponding to the amino acid acids from position 1 to position 18 in SEQ ID NO:42 (i.e., one amino acid deletion from the cytoplasmic domain). In embodiments, the truncated cytoplasmic domain has the amino acid sequence corresponding to the amino acid acids from position 1 to position 17 in SEQ ID NO:42 (i.e., two amino acid deletions from the cytoplasmic domain). In embodiments, the truncated cytoplasmic domain has the amino acid sequence corresponding to the amino acid acids from position 1 to position 16 in SEQ ID NO:42 (i.e., three amino acid deletions from the cytoplasmic domain). In embodiments, the truncated cytoplasmic domain has the amino acid sequence corresponding to the amino acid acids from position 1 to position 15 in SEQ ID NO:42 (i.e., four amino acid deletions from the cytoplasmic domain). In embodiments, the truncated cytoplasmic domain has the amino acid sequence corresponding to the amino acid acids from position 1 to position 14 in SEQ ID NO:42 (i.e., five amino acid deletions from the cytoplasmic domain). In embodiments, the truncated cytoplasmic domain has the amino acid sequence corresponding to the amino acid acids from position 1 to position 13 in SEQ ID NO:42 (i.e., six amino acid deletions from the cytoplasmic domain). In embodiments, the truncated cytoplasmic domain has the amino acid sequence corresponding to the amino acid acids from position 1 to position 12 in SEQ ID NO:42 (i.e., seven amino acid deletions from the cytoplasmic domain). In embodiments, the truncated cytoplasmic domain has the amino acid sequence corresponding to the amino acid acids from position 1 to position 11 in SEQ ID NO:42 (i.e., eight amino acid deletions from the cytoplasmic domain). In embodiments, the truncated cytoplasmic domain has the amino acid sequence corresponding to the amino acid acids from position 1 to position 10 in SEQ ID NO:42 (i.e., nine amino acid deletions from the cytoplasmic domain). In embodiments, the truncated cytoplasmic domain has the amino acid sequence corresponding to the amino acid acids from position 1 to position 9 in SEQ ID NO:42 (i.e., ten amino acid deletions from the cytoplasmic domain). In embodiments, the truncated cytoplasmic domain has the amino acid sequence corresponding to the amino acid acids from position 1 to position 8 in SEQ ID NO:42 (i.e., eleven amino acid deletions from the cytoplasmic domain). In embodiments, the truncated cytoplasmic domain has the amino acid sequence corresponding to the amino acid acids from position 1 to position 7 in SEQ ID NO:42 (i.e., twelve amino acid deletions from the cytoplasmic domain). In embodiments, the truncated cytoplasmic domain has the amino acid sequence corresponding to the amino acid acids from position 1 to position 6 in SEQ ID NO:42 (i.e., thirteen amino acid deletions from the cytoplasmic domain). In embodiments, the truncated cytoplasmic domain has the amino acid sequence corresponding to the amino acid acids from position 1 to position 5 in SEQ ID NO:42 (i.e., fourteen amino acid deletions from the cytoplasmic domain). In embodiments, the truncated cytoplasmic domain has the amino acid sequence corresponding to the amino acid acids from position 1 to position 4 in SEQ ID NO:42 (i.e., fifteen amino acid deletions from the cytoplasmic domain). In embodiments, the truncated cytoplasmic domain has the amino acid sequence corresponding to the amino acid acids from position 1 to position 3 in SEQ ID NO:42 (i.e., sixteen amino acid deletions from the cytoplasmic domain). In embodiments, the truncated cytoplasmic domain has the amino acid sequence corresponding to the amino acid acids from position 1 to position 2 in SEQ ID NO:42 (i.e., seventeen amino acid deletions from the cytoplasmic domain). In embodiments, the truncated cytoplasmic domain has the amino acid sequence corresponding to the amino acid acids at position 1 t in SEQ ID NO:42 (i.e., eighteen amino acid deletions from the cytoplasmic domain). In embodiments, the cytoplasmic domain is completely removed from the spike (S) protein.

In embodiments, the cytoplasmic domain comprises a deletion from 1 amino acid to about 18 amino acids. In embodiments, the cytoplasmic domain comprises a deletion from about 2 amino acids to about 18 amino acids. In embodiments, the cytoplasmic domain comprises a deletion from about 3 amino acids to about 18 amino acids. In embodiments, the cytoplasmic domain comprises a deletion from about 4 amino acids to about 18 amino acids. In embodiments, the cytoplasmic domain comprises a deletion from about 5 amino acids to about 18 amino acids. In embodiments, the cytoplasmic domain comprises a deletion from about 6 amino acids to about 18 amino acids. In embodiments, the cytoplasmic domain comprises a deletion from about 7 amino acids to about 18 amino acids. In embodiments, the cytoplasmic domain comprises a deletion from about 8 amino acids to about 18 amino acids. In embodiments, the cytoplasmic domain comprises a deletion from about 9 amino acids to about 18 amino acids. In embodiments, the cytoplasmic domain comprises a deletion from about 10 amino acids to about 18 amino acids. In embodiments, the cytoplasmic domain comprises a deletion from about 11 amino acids to about 18 amino acids. In embodiments, the cytoplasmic domain comprises a deletion from about 12 amino acids to about 18 amino acids. In embodiments, the cytoplasmic domain comprises a deletion from about 13 amino acids to about 18 amino acids. In embodiments, the cytoplasmic domain comprises a deletion from about 14 amino acids to about 18 amino acids. In embodiments, the cytoplasmic domain comprises a deletion from about 15 amino acids to about 18 amino acids. In embodiments, the cytoplasmic domain comprises a deletion from about 16 amino acids to about 18 amino acids. In embodiments, the cytoplasmic domain comprises a deletion from about 17 amino acids to about 18 amino acids. In embodiments, the truncation of the C-terminal cytoplasmic domain of the SARS-CoV-2 S protein results in an increased expression of the protein in comparison to the unmodified protein.

In embodiments, the cytoplasmic domain comprises a deletion from about 11 amino acids to about 17 amino acids. In embodiments, the cytoplasmic domain comprises a deletion from about 12 amino acids to about 16 amino acids. In embodiments, the cytoplasmic domain comprises a deletion from about 13 amino acids to about 15 amino acids. In embodiments, the cytoplasmic domain of the SARS-CoV-2 S protein comprises a deletion of 14 amino acids from its C-terminal end, the deleted sequence being SEQ ID NO:19. In embodiments, the truncation of the C-terminal cytoplasmic domain of the SARS-CoV-2 S protein results in an increased expression of the protein in comparison to the unmodified protein.

In embodiments the SARS-CoV-2 S protein further includes a truncated transmembrane domain. In embodiments, the SARS-CoV-2 spike (S) protein comprises a truncated transmembrane domain of SEQ ID NO:41 only when the cytoplasmic domain is not present in the SARS-CoV-2 spike (S) protein. In other words, a truncated transmembrane domain requires that the cytoplasmic domain is not present (i.e., all the amino acids are deleted from the cytoplasmic membrane in order for the transmembrane domain to be truncated).

In embodiments, the truncated transmembrane domain comprises a deletion of one, more than one, or all amino acids from the C-terminal end of a naturally-occurring S protein transmembrane domain. In embodiments, the truncated transmembrane domain comprises a deletion of at least 1 amino acids. In embodiments, the truncated transmembrane domain comprises a deletion of at least 2 amino acids. In embodiments, the truncated transmembrane domain comprises a deletion of at least 3 amino acids. In embodiments, the truncated transmembrane domain comprises a deletion of at least 4 amino acids. In embodiments, the truncated transmembrane domain comprises a deletion of at least 5 amino acids. In embodiments, the truncated transmembrane domain comprises a deletion of at least 6 amino acids. In embodiments, the truncated transmembrane domain comprises a deletion of at least 7 amino acids. In embodiments, the truncated transmembrane domain comprises a deletion of at least 8 amino acids. In embodiments, the truncated transmembrane domain comprises a deletion of at least 9 amino acids. In embodiments, the truncated transmembrane domain comprises a deletion of at least 10 amino acids. In embodiments, the truncated transmembrane domain comprises a deletion of at least 11 amino acids. In embodiments, the truncated transmembrane domain comprises a deletion of at least 12 amino acids. In embodiments, the truncated transmembrane domain comprises a deletion of at least 13 amino acids. In embodiments, the truncated transmembrane domain comprises a deletion of at least 14 amino acids. In embodiments, the truncated transmembrane domain comprises a deletion of at least 15 amino acids. In embodiments, the truncated transmembrane domain comprises a deletion of at least 16 amino acids. In embodiments, the truncated transmembrane domain comprises a deletion of at least 17 amino acids. In embodiments, the truncated transmembrane domain comprises a deletion of at least 18 amino acids. In embodiments, the truncated transmembrane domain comprises a deletion of at least 19 amino acids. In embodiments, the truncated transmembrane domain comprises a deletion of at least 20 amino acids. In embodiments, the truncated transmembrane domain comprises a deletion of at least 21 amino acids. In embodiments, the truncated transmembrane domain comprises a deletion of at least 22 amino acids. In embodiments, the truncated transmembrane domain comprises a deletion of at least 23 amino acids. In embodiments, the truncated transmembrane domain comprises a deletion of at least 24 amino acids. In embodiments, the truncated transmembrane domain comprises a deletion of at least 25 amino acids. In embodiments, the truncated transmembrane domain comprises a deletion of at least 26 amino acids. In embodiments, the truncated transmembrane domain comprises a deletion of at least 27 amino acids. In embodiments, the truncated transmembrane domain comprises a deletion of at least 28 amino acids. In embodiments, the truncated transmembrane domain comprises a deletion of at least 29 amino acids. In embodiments, the truncated transmembrane domain comprises a deletion of at least 30 amino acids. In embodiments, the truncated transmembrane domain comprises a deletion of at least 31 amino acids. In embodiments, the truncated transmembrane domain comprises a deletion of at least 32 amino acids. In embodiments, the truncated transmembrane domain comprises a deletion of at least 33 amino acids. In embodiments, the truncated transmembrane domain comprises a deletion of at least 34 amino acids. In embodiments, the truncated transmembrane domain comprises a deletion of at least 35 amino acids. In embodiments, the truncated transmembrane domain comprises a deletion of at least 36 amino acids. In embodiments, the truncated transmembrane domain comprises a deletion of at least 37 amino acids. In embodiments, the truncated transmembrane domain comprises a deletion of at least 38 amino acids. In embodiments, the truncated transmembrane domain comprises a deletion of at least 39 amino acids. In embodiments, the truncated transmembrane domain comprises a deletion of at least 40 amino acids. In embodiments, the truncated transmembrane domain comprises a deletion of at least 41 amino acids. In embodiments, the truncated transmembrane domain comprises a deletion of at least 42 amino acids. In embodiments, the truncated transmembrane domain comprises a deletion of at least 43 amino acids. In embodiments, the truncated transmembrane domain comprises a deletion of at least 44 amino acids. In embodiments, the truncated transmembrane domain comprises a deletion of at least 45 amino acids. In embodiments, the truncated transmembrane domain comprises a deletion of at least 46 amino acids. In embodiments, the transmembrane domain comprises a deletion of all the amino acids.

In embodiments, the truncated transmembrane domain has the amino acid sequence corresponding to the amino acids from position 1 to position 45 in SEQ ID NO:41 (i.e., one amino acid deletion from the transmembrane domain). In embodiments, the truncated transmembrane domain has the amino acid sequence corresponding to the amino acids from position 1 to position 44 in SEQ ID NO:40 (i.e., two amino acid deletions from the transmembrane domain). In embodiments, the truncated transmembrane domain has the amino acid sequence corresponding to the amino acids from position 1 to position 43 in SEQ ID NO:41 (i.e., three amino acid deletions from the transmembrane domain). In embodiments, the truncated transmembrane domain has the amino acid sequence corresponding to the amino acids from position 1 to position 42 in SEQ ID NO:41 (i.e., four amino acid deletions from the transmembrane domain). In embodiments, the truncated transmembrane domain has the amino acid sequence corresponding to the amino acids from position 1 to position 41 in SEQ ID NO:41 (i.e., five amino acid deletions from the transmembrane domain). In embodiments, the truncated transmembrane domain has the amino acid sequence corresponding to the amino acids from position 1 to position 40 in SEQ ID NO:41 (i.e., six amino acid deletions from the transmembrane domain). In embodiments, the truncated transmembrane domain has the amino acid sequence corresponding to the amino acids from position 1 to position 39 in SEQ ID NO:41 (i.e., seven amino acid deletions from the transmembrane domain). In embodiments, the truncated transmembrane domain has the amino acid sequence corresponding to the amino acids from position 1 to position 38 in SEQ ID NO:41 (i.e., eight amino acid deletions from the transmembrane domain). In embodiments, the truncated transmembrane domain has the amino acid sequence corresponding to the amino acids from position 1 to position 37 in SEQ ID NO:41 (i.e., nine amino acid deletions from the transmembrane domain). In embodiments, the truncated transmembrane domain has the amino acid sequence corresponding to the amino acids from position 1 to position 36 in SEQ ID NO:41 (i.e., ten amino acid deletions from the transmembrane domain). In embodiments, the truncated transmembrane domain has the amino acid sequence corresponding to the amino acids from position 1 to position 35 in SEQ ID NO:41 (i.e., eleven amino acid deletions from the transmembrane domain). In embodiments, the truncated transmembrane domain has the amino acid sequence corresponding to the amino acids from position 1 to position 34 in SEQ ID NO:41 (i.e., twelve amino acid deletions from the transmembrane domain). In embodiments, the truncated transmembrane domain has the amino acid sequence corresponding to the amino acids from position 1 to position 33 in SEQ ID NO:41 (i.e., thirteen amino acid deletions from the transmembrane domain). In embodiments, the truncated transmembrane domain has the amino acid sequence corresponding to the amino acids from position 1 to position 32 in SEQ ID NO:41 (i.e., fourteen amino acid deletions from the transmembrane domain). In embodiments, the truncated transmembrane domain has the amino acid sequence corresponding to the amino acids from position 1 to position 31 in SEQ ID NO:41 (i.e., fifteen amino acid deletions from the transmembrane domain). In embodiments, the truncated transmembrane domain has the amino acid sequence corresponding to the amino acids from position 1 to position 30 in SEQ ID NO:41 (i.e., sixteen amino acid deletions from the transmembrane domain). In embodiments, the truncated transmembrane domain has the amino acid sequence corresponding to the amino acids from position 1 to position 29 in SEQ ID NO:41 (i.e., seventeen amino acid deletions from the transmembrane domain). In embodiments, the truncated transmembrane domain has the amino acid sequence corresponding to the amino acids from position 1 to position 28 in SEQ ID NO:41 (i.e., eighteen amino acid deletions from the transmembrane domain). In embodiments, the truncated transmembrane domain has the amino acid sequence corresponding to the amino acids from position 1 to position 27 in SEQ ID NO:41 (i.e., nineteen amino acid deletions from the transmembrane domain). In embodiments, the truncated transmembrane domain has the amino acid sequence corresponding to the amino acids from position 1 to position 26 in SEQ ID NO:41 (i.e., twenty amino acid deletions from the transmembrane domain). In embodiments, the truncated transmembrane domain has the amino acid sequence corresponding to the amino acids from position 1 to position 25 in SEQ ID NO:41 (i.e., twenty-one amino acid deletions from the transmembrane domain). In embodiments, the truncated transmembrane domain has the amino acid sequence corresponding to the amino acids from position 1 to position 24 in SEQ ID NO:41 (i.e., twenty-two amino acid deletions from the transmembrane domain). In embodiments, the truncated transmembrane domain has the amino acid sequence corresponding to the amino acids from position 1 to position 23 in SEQ ID NO:41 (i.e., twenty-three amino acid deletions from the transmembrane domain). In embodiments, the truncated transmembrane domain has the amino acid sequence corresponding to the amino acids from position 1 to position 22 in SEQ ID NO:41 (i.e., twenty-four amino acid deletions from the transmembrane domain). In embodiments, the truncated transmembrane domain has the amino acid sequence corresponding to the amino acids from position 1 to position 21 in SEQ ID NO:41 (i.e., twenty-five amino acid deletions from the transmembrane domain). In embodiments, the truncated transmembrane domain has the amino acid sequence corresponding to the amino acids from position 1 to position 20 in SEQ ID NO:41 (i.e., twenty-six amino acid deletions from the transmembrane domain). In embodiments, the truncated transmembrane domain has the amino acid sequence corresponding to the amino acids from position 1 to position 19 in SEQ ID NO:41 (i.e., twenty-seven amino acid deletions from the transmembrane domain). In embodiments, the truncated transmembrane domain has the amino acid sequence corresponding to the amino acids from position 1 to position 18 in SEQ ID NO:41 (i.e., twenty-eight amino acid deletions from the transmembrane domain). In embodiments, the truncated transmembrane domain has the amino acid sequence corresponding to the amino acids from position 1 to position 17 in SEQ ID NO:41 (i.e., twenty-nine amino acid deletions from the transmembrane domain). In embodiments, the truncated transmembrane domain has the amino acid sequence corresponding to the amino acids from position 1 to position 16 in SEQ ID NO:41 (i.e., thirty amino acid deletions from the transmembrane domain). In embodiments, the truncated transmembrane domain has the amino acid sequence corresponding to the amino acids from position 1 to position 15 in SEQ ID NO:41 (i.e., thirty-one amino acid deletions from the transmembrane domain). In embodiments, the truncated transmembrane domain has the amino acid sequence corresponding to the amino acids from position 1 to position 14 in SEQ ID NO:41 (i.e., thirty-two amino acid deletions from the transmembrane domain). In embodiments, the truncated transmembrane domain has the amino acid sequence corresponding to the amino acids from position 1 to position 13 in SEQ ID NO:41 (i.e., thirty-three amino acid deletions from the transmembrane domain). In embodiments, the truncated transmembrane domain has the amino acid sequence corresponding to the amino acids from position 1 to position 12 in SEQ ID NO:41 (i.e., thirty-four amino acid deletions from the transmembrane domain). In embodiments, the truncated transmembrane domain has the amino acid sequence corresponding to the amino acids from position 1 to position 11 in SEQ ID NO:41 (i.e., thirty-five amino acid deletions from the transmembrane domain). In embodiments, the truncated transmembrane domain has the amino acid sequence corresponding to the amino acids from position 1 to position 10 in SEQ ID NO:41 (i.e., thirty-six amino acid deletions from the transmembrane domain). In embodiments, the truncated transmembrane domain has the amino acid sequence corresponding to the amino acids from position 1 to position 9 in SEQ ID NO:41 (i.e., thirty-seven amino acid deletions from the transmembrane domain). In embodiments, the truncated transmembrane domain has the amino acid sequence corresponding to the amino acids from position 1 to position 8 in SEQ ID NO:41 (i.e., thirty-eight amino acid deletions from the transmembrane domain). In embodiments, the truncated transmembrane domain has the amino acid sequence corresponding to the amino acids from position 1 to position 7 in SEQ ID NO:41 (i.e., thirty-nine amino acid deletions from the transmembrane domain). In embodiments, the truncated transmembrane domain has the amino acid sequence corresponding to the amino acids from position 1 to position 6 in SEQ ID NO:41 (i.e., forty amino acid deletions from the transmembrane domain). In embodiments, the truncated transmembrane domain has the amino acid sequence corresponding to the amino acids from position 1 to position 5 in SEQ ID NO:41 (i.e., forty-one amino acid deletions from the transmembrane domain). In embodiments, the truncated transmembrane domain has the amino acid sequence corresponding to the amino acids from position 1 to position 4 in SEQ ID NO:41 (i.e., forty-two amino acid deletions from the transmembrane domain). In embodiments, the truncated transmembrane domain has the amino acid sequence corresponding to the amino acids from position 1 to position 3 in SEQ ID NO:41 (i.e., forty-three amino acid deletions from the transmembrane domain). In embodiments, the truncated transmembrane domain has the amino acid sequence corresponding to the amino acids from position 1 to position 2 in SEQ ID NO:41 (i.e., forty-four amino acid deletions from the transmembrane domain). In embodiments, the truncated transmembrane domain has the amino acid sequence corresponding to the amino acid at position 1 in SEQ ID NO:40 (i.e., forty-five amino acid deletions from the transmembrane domain). In embodiments, the transmembrane domain is completely removed from the amino acid sequence of the SARS-CoV-2 spike (S) protein. When the transmembrane is a truncated transmembrane domain, then the cytoplasmic domain is not present in the protein.

In embodiments, the transmembrane domain comprises a deletion from 1 amino acid to about 45 amino acids. In embodiments, the transmembrane domain comprises a deletion from about 2 amino acids to about 45 amino acids. In embodiments, the transmembrane domain comprises a deletion from about 3 amino acids to about 45 amino acids. In embodiments, the transmembrane domain comprises a deletion from about 4 amino acids to about 45 amino acids. In embodiments, the transmembrane domain comprises a deletion from about 5 amino acids to about 45 amino acids. In embodiments, the transmembrane domain comprises a deletion from about 6 amino acids to about 45 amino acids. In embodiments, the transmembrane domain comprises a deletion from about 7 amino acids to about 45 amino acids. In embodiments, the transmembrane domain comprises a deletion from about 8 amino acids to about 45 amino acids. In embodiments, the transmembrane domain comprises a deletion from about 9 amino acids to about 45 amino acids. In embodiments, the transmembrane domain comprises a deletion from about 10 amino acids to about 45 amino acids. In embodiments, the transmembrane domain comprises a deletion from about 11 amino acids to about 45 amino acids. In embodiments, the transmembrane domain comprises a deletion from about 12 amino acids to about 45 amino acids. In embodiments, the transmembrane domain comprises a deletion from about 13 amino acids to about 45 amino acids. In embodiments, the transmembrane domain comprises a deletion from about 14 amino acids to about 45 amino acids. In embodiments, the transmembrane domain comprises a deletion from about 15 amino acids to about 45 amino acids. In embodiments, the transmembrane domain comprises a deletion from about 16 amino acids to about 45 amino acids. In embodiments, the transmembrane domain comprises a deletion from about 17 amino acids to about 45 amino acids. In embodiments, the transmembrane domain comprises a deletion from about 18 amino acids to about 45 amino acids. In embodiments, the transmembrane domain comprises a deletion from about 19 amino acids to about 45 amino acids. In embodiments, the transmembrane domain comprises a deletion from about 20 amino acids to about 45 amino acids. In embodiments, the transmembrane domain comprises a deletion from about 21 amino acids to about 45 amino acids. In embodiments, the transmembrane domain comprises a deletion from about 22 amino acids to about 45 amino acids. In embodiments, the transmembrane domain comprises a deletion from about 23 amino acids to about 45 amino acids. In embodiments, the transmembrane domain comprises a deletion from about 24 amino acids to about 45 amino acids. In embodiments, the transmembrane domain comprises a deletion from about 25 amino acids to about 45 amino acids. In embodiments, the transmembrane domain comprises a deletion from about 26 amino acids to about 45 amino acids. In embodiments, the transmembrane domain comprises a deletion from about 27 amino acids to about 45 amino acids. In embodiments, the transmembrane domain comprises a deletion from about 28 amino acids to about 45 amino acids. In embodiments, the transmembrane domain comprises a deletion from about 29 amino acids to about 45 amino acids. In embodiments, the transmembrane domain comprises a deletion from about 30 amino acids to about 45 amino acids. In embodiments, the transmembrane domain comprises a deletion from about 31 amino acids to about 45 amino acids. In embodiments, the transmembrane domain comprises a deletion from about 32 amino acids to about 45 amino acids. In embodiments, the transmembrane domain comprises a deletion from about 33 amino acids to about 45 amino acids. In embodiments, the transmembrane domain comprises a deletion from about 34 amino acids to about 45 amino acids. In embodiments, the transmembrane domain comprises a deletion from about 35 amino acids to about 45 amino acids. In embodiments, the transmembrane domain comprises a deletion from about 36 amino acids to about 45 amino acids. In embodiments, the transmembrane domain comprises a deletion from about 37 amino acids to about 45 amino acids. In embodiments, the transmembrane domain comprises a deletion from about 38 amino acids to about 45 amino acids. In embodiments, the transmembrane domain comprises a deletion from about 39 amino acids to about 45 amino acids. In embodiments, the transmembrane domain comprises a deletion from about 40 amino acids to about 45 amino acids. In embodiments, the transmembrane domain comprises a deletion from about 41 amino acids to about 45 amino acids. In embodiments, the transmembrane domain comprises a deletion from about 42 amino acids to about 45 amino acids. In embodiments, the transmembrane domain comprises a deletion from about 43 amino acids to about 45 amino acids. In embodiments, the transmembrane domain comprises a deletion from about 44 amino acids to about 45 amino acids.

In embodiments, the SARS-CoV-2 S protein does not include a S1/S2 protease cleavage site or the S1/S2 protease cleavage site is replaced with a peptide linker. In embodiments, the SARS-CoV-2 S protein does not include a S2′ protease cleavage site or the S2′ protease cleavage site is replaced with a peptide linker. In embodiments, the SARS-CoV-2 S protein does not include a S1/S2 protease cleavage site and the a S2′ protease cleavage site is replaced with a peptide linker. In embodiments, the SARS-CoV-2 S protein does not include a S2′ protease cleavage site and the a S1/S2 protease cleavage site is replaced with a peptide linker.

In embodiments, the SARS-CoV-2 S protein does not include a S1/S2 protease cleavage site and/or a S2′ protease cleavage site. In embodiments, the SARS-CoV-2 S protein does not include a S1/S2 protease cleavage site. In embodiments, the SARS-CoV-2 S protein does not include a S2′ protease cleavage site. In embodiments, the SARS-CoV-2 S protein does not include a S1/S2 protease cleavage site or a S2′ protease cleavage site.

In embodiments, the S1/S2 protease cleavage site and/or S2′ protease cleavage site is replaced with a peptide linker. In embodiments, the S1/S2 protease cleavage site is replaced with a peptide linker. In embodiments, the S2′ protease cleavage site is replaced with a peptide linker. In embodiments, the S1/S2 protease cleavage site and the S2′ cleavage site are replaced with a peptide linker. In embodiments, the linker comprises from 1 to about 50 amino acids. In embodiments, the linker comprises from 1 to about 40 amino acids. In embodiments, the linker comprises from 1 to about 30 amino acids. In embodiments, the linker comprises from 1 to about 25 amino acids. In embodiments, the linker comprises from 1 to about 20 amino acids. In embodiments, the linker comprises from 1 to about 18 amino acids. In embodiments, the linker comprises from 1 to about 16 amino acids. In embodiments, the linker comprises from 1 to about 15 amino acids. In embodiments, the linker comprises from 1 to about 14 amino acids. In embodiments, the linker comprises from 3 to about 14 amino acids. In embodiments, the linker comprises from 4 to about 20 amino acids. In embodiments, the linker comprises from 9 to about 15 amino acids. In embodiments, the peptide linker is an amino acid sequence comprising a majority of glycine amino acids. In embodiments, the amino acid sequence comprises a majority of serine amino acids. In embodiments, the linker consists of serine and glycine amino acids. In embodiments, the linker comprises from 1 to about 25 amino acids, wherein the amino acids consist of serine and glycine. In embodiments, the linker comprises from 1 to about 20 amino acids, wherein the amino acids consist of serine and glycine. In embodiments, the linker comprises from 1 to about 18 amino acids, wherein the amino acids consist of serine and glycine. In embodiments, the linker comprises from 1 to about 16 amino acids, wherein the amino acids consist of serine and glycine. In embodiments, the linker comprises from 1 to about 15 amino acids, wherein the amino acids consist of serine and glycine. In embodiments, the linker comprises from 1 to about 14 amino acids, wherein the amino acids consist of serine and glycine. In embodiments, the linker comprises from 3 to about 14 amino acids, wherein the amino acids consist of serine and glycine. In embodiments, the linker comprises from 4 to about 20 amino acids, wherein the amino acids consist of serine and glycine. In embodiments, the linker comprises from 9 to about 15 amino acids, wherein the amino acids consist of serine and glycine.

In embodiments, the linker comprises at least 1 amino acid. In embodiments, the linker comprises at least 2 amino acids. In embodiments, the linker comprises at least 3 amino acids. In embodiments, the linker comprises at least 4 amino acids. In embodiments, the linker comprises at least 5 amino acids. In embodiments, the linker comprises at least 6 amino acids. In embodiments, the linker comprises at least 7 amino acids. In embodiments, the linker comprises at least 8 amino acids. In embodiments, the linker comprises at least 9 amino acids. In embodiments, the linker comprises at least 10 amino acids. In embodiments, the linker comprises at least 11 amino acids. In embodiments, the linker comprises at least 12 amino acids. In embodiments, the linker comprises at least 13 amino acids. In embodiments, the linker comprises at least 14 amino acids. In embodiments, the linker comprises at least 15 amino acids. In embodiments, the linker comprises at least 16 amino acids. In embodiments, the linker comprises at least 17 amino acids. In embodiments, the linker comprises at least 18 amino acids. In embodiments, the linker comprises at least 19 amino acids. In embodiments, the linker comprises at least 20 amino acids. In embodiments, the linker comprises about 1 amino acid. In embodiments, the linker comprises about 2 amino acids. In embodiments, the linker comprises about 3 amino acids. In embodiments, the linker comprises about 4 amino acids. In embodiments, the linker comprises about 5 amino acids. In embodiments, the linker comprises about 6 amino acids. In embodiments, the linker comprises about 7 amino acids. In embodiments, the linker comprises about 8 amino acids. In embodiments, the linker comprises about 9 amino acids. In embodiments, the linker comprises about 10 amino acids. In embodiments, the linker comprises about 11 amino acids. In embodiments, the linker comprises about 12 amino acids. In embodiments, the linker comprises about 13 amino acids. In embodiments, the linker comprises about 14 amino acids. In embodiments, the linker comprises about 15 amino acids. In embodiments, the linker comprises about 16 amino acids. In embodiments, the linker comprises about 17 amino acids. In embodiments, the linker comprises about 18 amino acids. In embodiments, the linker comprises about 19 amino acids. In embodiments, the linker comprises about 20 amino acids.

In embodiments, the S1/S2 protease cleavage site and/or S2′ protease cleavage site is replaced with a peptide linker. In embodiments, the S1/S2 protease cleavage site is replaced with a peptide linker. In embodiments, the S2′ protease cleavage site is replaced with a peptide linker. In embodiments, the S1/S2 protease cleavage site and the S2′ cleavage site are replaced with a peptide linker. In embodiments, the linker consists of serine and glycine amino acids. In embodiments, the linker is -(G₄S)ₓ—, where x is an integer from 1 to 10. In embodiments, the linker is -(G₄S)ₓ—, where x is an integer from 1 to 6. In embodiments, the linker is -(G₄S)ₓ—, where x is an integer from 1 to 3. In embodiments, the linker is -(G₃S)ₓ—, where x is an integer from 1 to 10. In embodiments, the linker is -(G₃S)ₓ—, where x is an integer from 1 to 6. In embodiments, the linker is -(G₃S)ₓ—, where x is an integer from 1 to 3. In embodiments, the linker is —S(G₄S)ₓ—, where x is an integer from 1 to 10. In embodiments, the linker is —S(G₄S)ₓ—, where x is an integer from 1 to 6. In embodiments, the linker is —S(G₄S)ₓ—, where x is an integer from 1 to 3. In embodiments, the linker is —S(G₃S)ₓ—, where x is an integer from 1 to 10. In embodiments, the linker is —S(G₃S)ₓ—, where x is an integer from 1 to 6. In embodiments, the linker is —S(G₃S)ₓ—, where x is an integer from 1 to 3. In embodiments, the linker is -(G₄S)ₓ— where x is an integer from 1 to 10. In embodiments, the linker is -(G₄S)ₓ— where x is an integer from 1 to 6. In embodiments, the linker is -(G₄S)ₓ— where x is an integer from 1 to 4. In embodiments, the linker is -(G₄S)ₓ— where x is an integer from 1 to 3. In embodiments, the linker is -(G₃S)ₓ— where x is an integer from 1 to 10. In embodiments, the linker is -(G₃S)ₓ— where x is an integer from 1 to 6. In embodiments, the linker is -(G₃S)ₓ— where x is an integer from 1 to 4. In embodiments, the linker is -(G₃S)ₓ— where x is an integer from 1 to 3.

In embodiments, the linker comprises one or more GGGGS (SEQ ID NO:22) (G₄S) repeats. In embodiments, the linker comprises at least one G₄S repeat. In embodiments, the linker comprises at least two G₄S repeats. In embodiments, the linker comprises at least three G₄S repeats. In embodiments, the linker is a -G₄S— peptide linker. In embodiments, the linker is a (G₄S)₂ peptide linker. In embodiments, the linker is a (G₄S)₃ peptide linker having the sequence of GGGGSGGGGSGGGGS (SEQ ID NO:23). In embodiments, the linker is a (G₄S)₄ peptide linker. In embodiments, the sequence of the linker is SGGGGSGGGGSGGGGS (SEQ ID NO:24). In embodiments, the linker comprises one or more GGGS (SEQ ID NO:25) (G₃S) repeats. In embodiments, the linker comprises at least one G₃S repeat. In embodiments, the linker comprises at least two G₃S repeats. In embodiments, the linker comprises at least three G₃S repeats. In embodiments, the linker is a -G₃S— peptide linker. In embodiments, the linker is a (G₃S)₂ peptide linker. In embodiments, the linker is a (G₃S)₃ peptide linker having the sequence GGGSGGGSGGGS (SEQ ID NO:26). In embodiments, the linker is a (G₃S)₄ peptide linker. In embodiments, the sequence of the linker is SGGGSGGGSGGGS (SEQ ID NO:27). The skilled artisan will appreciate that S is serine and G is glycine.

In embodiments, the SARS-CoV-2 protein further comprises a human MIP3α protein. In embodiments, the human MIP3α protein has SEQ ID NO:29. In embodiments, the human MIP3α protein is located before (at the N-terminus of) the spike (S) protein.

Provided herein are proteins comprising the amino acid sequence of formula (I): R¹-L¹-R²-L²-R³-R⁴-R⁵. Exemplary proteins of formula (I) include SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

Provided herein are nucleic acids encoding the amino acid sequence of formula (I): R¹-L¹-R²-L²-R³-R⁴-R⁵. Provided herein are nucleic acids encoding the sequence of formula (I): R¹-L¹-R²-L²-R³-R⁴-R⁵ and a human MIP3α protein. Provided herein are nucleic acids comprising SEQ ID NO:28 and encoding the amino acid sequence of formula (I): R¹-L¹-R²-L²-R³-R⁴-R⁵. Provided herein are nucleic acids encoding SEQ ID NO:29 and encoding the sequence of amino acid formula (I): R¹-L¹-R²-L²-R³-R⁴-R¹. Provided herein are nucleic acids encoding SEQ ID NO:17 and encoding the sequence of amino acid formula (I): R¹-L¹-R²-L²-R³-R⁴-R⁵. Provided herein are plasmids comprising a nucleic acid encoding the sequence of formula (I): R¹-LI-R²-L²-R³-R⁴-R⁵.

Provided herein are plasmids comprising a nucleic acid encoding the amino acid sequence of formula (I): R¹-LI-R²-L²-R³-R⁴-R⁵ and a human MIP3α protein. Provided herein are plasmids comprising a nucleic acid comprising SEQ ID NO:28 and encoding the sequence of formula (I): R¹-LI-R²-L²-R³-R⁴-R⁵. Provided herein are plasmids comprising a nucleic acid encoding SEQ ID NO:29 and encoding the amino acid sequence of formula (I): R¹-LI-R²-L²-R³-R⁴-R⁵. Provided herein are plasmids comprising a nucleic acid encoding SEQ ID NO:17 and encoding the amino acid sequence of formula (I): R¹-L¹-R²-L²-R³-R⁴-R⁵.

In embodiments, R¹ is an amino acid sequence having at least 90% sequence identity to SEQ ID NO:46; R² is an amino acid sequence having at least 90% sequence identity to SEQ ID NO:47; R³ is an amino acid sequence having at least 90% sequence identity to SEQ ID NO:48. R¹ is an amino acid sequence having at least 95% sequence identity to SEQ ID NO:46; R² is an amino acid sequence having at least 95% sequence identity to SEQ ID NO:47; and R³ is an amino acid sequence having at least 95% sequence identity to SEQ ID NO:48. R¹ is an amino acid sequence having SEQ ID NO:46; R² is an amino acid sequence having SEQ ID NO:47; and R³ is an amino acid sequence having SEQ ID NO:48.

In embodiments, R⁴ is an amino acid sequence having at least 90% sequence identity to SEQ ID NO:41. In embodiments, R⁴ is an amino acid sequence having at least 95% sequence identity to SEQ ID NO:41. In embodiments, R⁴ is an amino acid sequence having SEQ ID NO:41. In embodiments, R⁵ is absent. In embodiments, R⁵ is a truncated cytoplasmic domain, as described herein, including embodiments thereof.

In embodiments, R⁴ is absent and R⁵ is absent. In embodiments, R⁴ is a truncated transmembrane domain, as described herein, including embodiments thereof, and R⁵ is absent.

In embodiments, L¹ and L² are not simultaneously SEQ ID NO:20 and SEQ ID NO:21, respectively. In embodiments, L¹ is SEQ ID NO:20, and L² is absent, —(S)_{y}(G₄S)ₓ—, or —(S)_{y}(G₃S)ₓ—; where y is 0 or 1, and x is an integer from 1 to 10. In embodiments, L¹ is SEQ ID NO:20, and L² is —S(G₄S)ₓ—, -(G₄S)ₓ—, —S(G₃S)ₓ—, or -(G₃S)ₓ—; where x is an integer from 1 to 10.

In embodiments, L¹ is SEQ ID NO:20, and L² is absent. In embodiments, L¹ is absent, —S(G₄S)ₓ—, -(G₄S)ₓ—, —S(G₃S)ₓ—, or -(G₃S)ₓ—; where x is an integer from 1 to 10; and L² is SEQ ID NO:21. In embodiments, L¹ is absent and L² is SEQ ID NO:21. In embodiments, L¹ is —S(G₄S)ₓ—, -(G₄S)ₓ—, —S(G₃S)ₓ—, or -(G₃S)ₓ—; where x is an integer from 1 to 10; and L² is SEQ ID NO:21. In embodiments, L¹ is absent, —S(G₄S)ₓ—, -(G₄S)ₓ—, —S(G₃S)ₓ—, or -(G₃S)ₓ—; where x is an integer from 1 to 10; and L² is absent, —S(G₄S)ₓ—, -(G₄S)ₓ—, —S(G₃S)ₓ—, or -(G₃S)ₓ—; where x is an integer from 1 to 10. In embodiments, L¹ is absent and L² is —S(G₄S)ₓ—, -(G₄S)ₓ—, —S(G₃S)ₓ—, or -(G₃S)ₓ—; where x is an integer from 1 to 10. In embodiments, L¹ is —S(G₄S)ₓ—, -(G₄S)ₓ—, —S(G₃S)ₓ—, or -(G₃S)ₓ—; where x is an integer from 1 to 10; and L² is absent. In embodiments, L¹ and L² are both absent. In embodiments, L¹ is —S(G₄S)ₓ—, -(G₄S)ₓ—, —S(G₃S)ₓ—, or -(G₃S)ₓ—; where x is an integer from 1 to 10; and L² is —S(G₄S)ₓ—, -(G₄S)ₓ—, —S(G₃S)ₓ—, or -(G₃S)ₓ—; where x is an integer from 1 to 10. In embodiments, L¹ is -(G₄S)ₓ— or -(G₃S)ₓ—; where x is an integer from 1 to 10; and L² is -(G₄S)ₓ— or -(G₃S)ₓ—; where x is an integer from 1 to 10. In embodiments, L¹ is -(G₄S)ₓ— where x is an integer from 1 to 10; and L² is -(G₄S)ₓ— where x is an integer from 1 to 10. In embodiments, L¹ is -(G₃S)ₓ— where x is an integer from 1 to 10; and L² is -(G₃S)ₓ— where x is an integer from 1 to 10. In embodiments, x is an integer from 1 to 9. In embodiments, x is an integer from 1 to 8. In embodiments, x is an integer from 1 to 7. In embodiments, x is an integer from 1 to 6. In embodiments, x is an integer from 1 to 5. In embodiments, x is an integer from 1 to 4. In embodiments, x is an integer from 1 to 3. In embodiments, x is 1. In embodiments, x is 2. In embodiments, x is 3. In embodiments, x is 4. In embodiments, x is 5. In embodiments, x is 6.

In embodiments of the proteins comprising the amino acid sequence of formula (I): R¹-L¹-R²-L²-R³-R⁴-R⁵, the protein further comprises an amino acid sequence of a human MIP3α protein. In embodiments, the human MIP3α protein has SEQ ID NO:29. In embodiments, the human MIP3α protein has an amino acid sequence with at least 90% sequence identity to SEQ ID NO:17. In embodiments, the human MIP3α protein has an amino acid sequence with at least 95% sequence identity to SEQ ID NO:17. In embodiments, the human MIP3α protein has SEQ ID NO:17. In embodiments, the human MIP3α protein is located before R¹ (e.g., at the N-terminus of the protein).

In embodiments of the proteins comprising the amino acid sequence of formula (I): R¹-L¹-R²-L²-R³-R⁴-R⁵, the protein further comprises an amino acid sequence having SEQ ID NO:43. In embodiments, SEQ ID NO:43 is located before R¹ at the N-terminus of the protein. In embodiments, SEQ ID NO:43 is located after the human MIP3α protein and before R¹.

In embodiments, the disclosure provides a SARS-CoV-2 spike (S) protein comprising a truncated cytoplasmic domain. In embodiments, the SARS-CoV-2 spike (S) protein has at least 85% sequence identity to SEQ ID NO:4. In embodiments, the SARS-CoV-2 spike (S) protein has at least 90% sequence identity to SEQ ID NO:4. In embodiments, the SARS-CoV-2 spike (S) protein has at least 92% sequence identity to SEQ ID NO:4. In embodiments, the SARS-CoV-2 spike (S) protein has at least 94% sequence identity to SEQ ID NO:4. In embodiments, the SARS-CoV-2 spike (S) protein has at least 95% sequence identity to SEQ ID NO:4. In embodiments, the SARS-CoV-2 spike (S) protein has at least 96% sequence identity to SEQ ID NO:4. In embodiments, the SARS-CoV-2 spike (S) protein has at least 98% sequence identity to SEQ ID NO:4. In embodiments, the SARS-CoV-2 spike (S) protein has SEQ ID NO:4. In embodiments, the SARS-CoV-2 spike (S) protein has at least 90% sequence identity to SEQ ID NO:45. In embodiments, the SARS-CoV-2 spike (S) protein has at least 92% sequence identity to SEQ ID NO:45. In embodiments, the SARS-CoV-2 spike (S) protein has at least 94% sequence identity to SEQ ID NO:45. In embodiments, the SARS-CoV-2 spike (S) protein has at least 95% sequence identity to SEQ ID NO:45. In embodiments, the SARS-CoV-2 spike (S) protein has at least 96% sequence identity to SEQ ID NO:45. In embodiments, the SARS-CoV-2 spike (S) protein has at least 98% sequence identity to SEQ ID NO:45. In embodiments, the SARS-CoV-2 spike (S) protein has SEQ ID NO:45. In embodiments, the SARS-CoV-2 spike (S) protein has one or more mutations corresponding to, or aligning with, L5, L54, S221, V367, G476, S477, V483, D614, P681, A845, T859, or P1263 in SEQ ID NO:4. In embodiments, the SARS-CoV-2 spike (S) protein comprises a D614G mutation corresponding to or aligning with D614 of the amino acid sequence set forth as SEQ ID NO:44. In embodiments, the S1/S2 protease cleavage site in the SARS-CoV-2 spike (S) protein is absent or replaced with a peptide linker. In embodiments, the S2′ protease cleavage site in the SARS-CoV-2 spike (S) protein is absent or replaced with a peptide linker. In embodiments, the S1/S2 protease cleavage site is replaced with a peptide linker and the S2′ protease cleavage site is replaced with a peptide linker. In embodiments, the peptide linker is any described herein. In embodiments, the peptide linker is —S(G₄S)ₓ—, -(G₄S)ₓ—, —S(G₃S)ₓ—, or -(G₃S)ₓ—; wherein x is an integer from 1 to 10. In embodiments, the peptide linker is -(G₄S)₃—. In embodiments, the S1/S2 protease cleavage site in the SARS-CoV-2 spike (S) protein is absent and the S2′ protease cleavage site in the SARS-CoV-2 spike (S) protein is absent. In embodiments, the SARS-CoV-2 spike (S) protein further comprises a human MIP3α protein.

In embodiments, the disclosure provides a SARS-CoV-2 spike (S) protein comprising a truncated transmembrane domain; wherein the protein does not comprise the amino acid sequence of SEQ ID NO:42. In embodiments, the SARS-CoV-2 spike (S) protein has at least 85% sequence identity to SEQ ID NO:4. In embodiments, the SARS-CoV-2 spike (S) protein has at least 90% sequence identity to SEQ ID NO:4. In embodiments, the SARS-CoV-2 spike (S) protein has at least 92% sequence identity to SEQ ID NO:4. In embodiments, the SARS-CoV-2 spike (S) protein has at least 94% sequence identity to SEQ ID NO:4. In embodiments, the SARS-CoV-2 spike (S) protein has at least 95% sequence identity to SEQ ID NO:4. In embodiments, the SARS-CoV-2 spike (S) protein has at least 96% sequence identity to SEQ ID NO:4. In embodiments, the SARS-CoV-2 spike (S) protein has at least 98% sequence identity to SEQ ID NO:4. In embodiments, the SARS-CoV-2 spike (S) protein has SEQ ID NO:4. In embodiments, the SARS-CoV-2 spike (S) protein has at least 90% sequence identity to SEQ ID NO:45. In embodiments, the SARS-CoV-2 spike (S) protein has at least 92% sequence identity to SEQ ID NO:45. In embodiments, the SARS-CoV-2 spike (S) protein has at least 94% sequence identity to SEQ ID NO:45. In embodiments, the SARS-CoV-2 spike (S) protein has at least 95% sequence identity to SEQ ID NO:45. In embodiments, the SARS-CoV-2 spike (S) protein has at least 96% sequence identity to SEQ ID NO:45. In embodiments, the SARS-CoV-2 spike (S) protein has at least 98% sequence identity to SEQ ID NO:45. In embodiments, the SARS-CoV-2 spike (S) protein has SEQ ID NO:45. In embodiments, the SARS-CoV-2 spike (S) protein has one or more mutations corresponding to, or aligning with, L5, L54, S221, V367, G476, S477, V483, D614, P681, A845, T859, or P1263 in SEQ ID NO:4. In embodiments, the SARS-CoV-2 spike (S) protein comprises a D614G mutation corresponding to or aligning with D614 of the amino acid sequence set forth as SEQ ID NO:44. In embodiments, the S1/S2 protease cleavage site in the SARS-CoV-2 spike (S) protein is absent or replaced with a peptide linker. In embodiments, the S2′ protease cleavage site in the SARS-CoV-2 spike (S) protein is absent or replaced with a peptide linker. In embodiments, the S1/S2 protease cleavage site is replaced with a peptide linker and the S2′ protease cleavage site is replaced with a peptide linker. In embodiments, the peptide linker is any described herein. In embodiments, the peptide linker is —S(G₄S)ₓ—, -(G₄S)ₓ—, —S(G₃S)ₓ—, or -(G₃S)ₓ—; wherein x is an integer from 1 to 10. In embodiments, the peptide linker is -(G₄S)₃—. In embodiments, the S1/S2 protease cleavage site in the SARS-CoV-2 spike (S) protein is absent and the S2′ protease cleavage site in the SARS-CoV-2 spike (S) protein is absent. In embodiments, the SARS-CoV-2 spike (S) protein further comprises a human MIP3α protein.

In embodiments, the disclosure provides a SARS-CoV-2 spike (S) protein having at least 85% sequence identity to SEQ ID NO:4 or has at least 85% sequence identity to SEQ ID NO:45; provided that SEQ ID NO:4 and SEQ ID NO:45 do not comprise the amino acid sequence of SEQ ID NO:40. In embodiments, the SARS-CoV-2 spike (S) protein has at least 90% sequence identity to SEQ ID NO:4. In embodiments, the SARS-CoV-2 spike (S) protein has at least 92% sequence identity to SEQ ID NO:4. In embodiments, the SARS-CoV-2 spike (S) protein has at least 94% sequence identity to SEQ ID NO:4. In embodiments, the SARS-CoV-2 spike (S) protein has at least 95% sequence identity to SEQ ID NO:4. In embodiments, the SARS-CoV-2 spike (S) protein has at least 96% sequence identity to SEQ ID NO:4. In embodiments, the SARS-CoV-2 spike (S) protein has at least 98% sequence identity to SEQ ID NO:4. In embodiments, the SARS-CoV-2 spike (S) protein has SEQ ID NO:4. In embodiments, the SARS-CoV-2 spike (S) protein has at least 90% sequence identity to SEQ ID NO:45. In embodiments, the SARS-CoV-2 spike (S) protein has at least 92% sequence identity to SEQ ID NO:45. In embodiments, the SARS-CoV-2 spike (S) protein has at least 94% sequence identity to SEQ ID NO:45. In embodiments, the SARS-CoV-2 spike (S) protein has at least 95% sequence identity to SEQ ID NO:45. In embodiments, the SARS-CoV-2 spike (S) protein has at least 96% sequence identity to SEQ ID NO:45. In embodiments, the SARS-CoV-2 spike (S) protein has at least 98% sequence identity to SEQ ID NO:45. In embodiments, the SARS-CoV-2 spike (S) protein has SEQ ID NO:45. In embodiments, the SARS-CoV-2 spike (S) protein has one or more mutations corresponding to, or aligning with, L5, L54, S221, V367, G476, S477, V483, D614, P681, A845, T859, or P1263 in SEQ ID NO:4. In embodiments, the SARS-CoV-2 spike (S) protein comprises a D614G mutation corresponding to or aligning with D614 of the amino acid sequence set forth as SEQ ID NO:44. In embodiments, the S1/S2 protease cleavage site in the SARS-CoV-2 spike (S) protein is absent or replaced with a peptide linker. In embodiments, the S2′ protease cleavage site in the SARS-CoV-2 spike (S) protein is absent or replaced with a peptide linker. In embodiments, the S1/S2 protease cleavage site is replaced with a peptide linker and the S2′ protease cleavage site is replaced with a peptide linker. In embodiments, the peptide linker is any described herein. In embodiments, the peptide linker is —S(G₄S)ₓ—, -(G₄S)ₓ—, —S(G₃S)ₓ—, or -(G₃S)ₓ—; wherein x is an integer from 1 to 10. In embodiments, the peptide linker is -(G₄S)₃—. In embodiments, the S1/S2 protease cleavage site in the SARS-CoV-2 spike (S) protein is absent and the S2′ protease cleavage site in the SARS-CoV-2 spike (S) protein is absent. In embodiments, the SARS-CoV-2 spike (S) protein further comprises a human MIP3α protein.

In another aspect is provided a nucleic acid encoding the SARS-CoV-2 S protein as described herein. In embodiments, the nucleic acid encoding the SARS-CoV-2 S protein further encodes a different protein. In embodiments, the nucleic acid encoding the SARS-CoV-2 S protein further encodes a human protein. In embodiments, the nucleic acid encoding the SARS-CoV-2 S protein further encodes a human MIP3α protein. In another aspect is provided a nucleic acid encoding SARS-CoV-2 S protein and a human MIP3α protein.

In embodiments, the nucleic acid is a single stranded nucleic acid. In embodiments, the nucleic acid is a double stranded nucleic acid. In embodiments, the nucleic acid is a plasmid.

In embodiments, the plasmid includes a promoter driving the expression of the SARS-CoV-2 S protein. In embodiments, the plasmid includes an activatable promoter driving the expression of the SARS-CoV-2 S protein. In embodiments, the plasmid includes a ubiquitous promoter driving the expression of the SARS-CoV-2 S protein. In embodiments, the promoter is a human promoter. In embodiments, the promoter is non-human promoter. In embodiments, the promoter is a viral promoter. In embodiments, the activatable promoter is a human promoter. In embodiments, the activatable promoter is non-human promoter. In embodiments, the activatable promoter is a viral promoter. In embodiments, the ubiquitous promoter is a human promoter. In embodiments, the ubiquitous promoter is non-human promoter. In embodiments, the ubiquitous promoter is a viral promoter. In embodiments, the ubiquitous promoter is a CMV promoter. In embodiments, a polyadenylation signal is located downstream of the sequence encoding the SARS-CoV-2 S protein. In embodiments, the polyadenylation signal is a rabbit β-globulin polyadenylation signal.

Provided herein are proteins having at least 75% sequence identity, at least 80% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to any one of SEQ ID NO:2, SEQ ID NO:4, SEQ ID NO:6, SEQ ID NO:8, SEQ ID NO:10, SEQ ID NO:12, SEQ ID NO:14, or SEQ ID NO:16. In embodiments, the proteins do not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:2. In embodiments, the protein has at least 85% sequence identity to SEQ ID NO:2. In embodiments, the protein has at least 90% sequence identity to SEQ ID NO:2. In embodiments, the protein has at least 91% sequence identity to SEQ ID NO:2. In embodiments, the protein has at least 92% sequence identity to SEQ ID NO:2. In embodiments, the protein has at least 93% sequence identity to SEQ ID NO:2. In embodiments, the protein has at least 94% sequence identity to SEQ ID NO:2. In embodiments, the protein has at least 95% sequence identity to SEQ ID NO:2. In embodiments, the protein has at least 96% sequence identity to SEQ ID NO:2. In embodiments, the protein has at least 97% sequence identity to SEQ ID NO:2. In embodiments, the protein has at least 98% sequence identity to SEQ ID NO:2. In embodiments, the protein has at least 99% sequence identity to SEQ ID NO:2. In embodiments, the protein has SEQ ID NO:2. In embodiments, the protein is SEQ ID NO:2. In embodiments, SEQ ID NO:2 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:4. In embodiments, the protein has at least 85% sequence identity to SEQ ID NO:4. In embodiments, the protein has at least 90% sequence identity to SEQ ID NO:4. In embodiments, the protein has at least 91% sequence identity to SEQ ID NO:4. In embodiments, the protein has at least 92% sequence identity to SEQ ID NO:4. In embodiments, the protein has at least 93% sequence identity to SEQ ID NO:4. In embodiments, the protein has at least 94% sequence identity to SEQ ID NO:4. In embodiments, the protein has at least 95% sequence identity to SEQ ID NO:4. In embodiments, the protein has at least 96% sequence identity to SEQ ID NO:4. In embodiments, the protein has at least 97% sequence identity to SEQ ID NO:4. In embodiments, the protein has at least 98% sequence identity to SEQ ID NO:4. In embodiments, the protein has at least 99% sequence identity to SEQ ID NO:4. In embodiments, the protein has SEQ ID NO:4. In embodiments, the protein is SEQ ID NO:4. In embodiments, SEQ ID NO:4 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:6. In embodiments, the protein has at least 85% sequence identity to SEQ ID NO:6. In embodiments, the protein has at least 90% sequence identity to SEQ ID NO:6. In embodiments, the protein has at least 91% sequence identity to SEQ ID NO:6. In embodiments, the protein has at least 92% sequence identity to SEQ ID NO:6. In embodiments, the protein has at least 93% sequence identity to SEQ ID NO:6. In embodiments, the protein has at least 94% sequence identity to SEQ ID NO:6. In embodiments, the protein has at least 95% sequence identity to SEQ ID NO:6. In embodiments, the protein has at least 96% sequence identity to SEQ ID NO:6. In embodiments, the protein has at least 97% sequence identity to SEQ ID NO:6. In embodiments, the protein has at least 98% sequence identity to SEQ ID NO:6. In embodiments, the protein has at least 99% sequence identity to SEQ ID NO:6. In embodiments, the protein has SEQ ID NO:6. In embodiments, the protein is SEQ ID NO:6. In embodiments, SEQ ID NO:6 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:8. In embodiments, the protein has at least 85% sequence identity to SEQ ID NO:8. In embodiments, the protein has at least 90% sequence identity to SEQ ID NO:8. In embodiments, the protein has at least 91% sequence identity to SEQ ID NO:8. In embodiments, the protein has at least 92% sequence identity to SEQ ID NO:8. In embodiments, the protein has at least 93% sequence identity to SEQ ID NO:8. In embodiments, the protein has at least 94% sequence identity to SEQ ID NO:8. In embodiments, the protein has at least 95% sequence identity to SEQ ID NO:8. In embodiments, the protein has at least 96% sequence identity to SEQ ID NO:8. In embodiments, the protein has at least 97% sequence identity to SEQ ID NO:8. In embodiments, the protein has at least 98% sequence identity to SEQ ID NO:8. In embodiments, the protein has at least 99% sequence identity to SEQ ID NO:8. In embodiments, the protein has SEQ ID NO:2. In embodiments, the protein is SEQ ID NO:8. In embodiments, SEQ ID NO:8 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:10. In embodiments, the protein has at least 85% sequence identity to SEQ ID NO:10. In embodiments, the protein has at least 90% sequence identity to SEQ ID NO:10. In embodiments, the protein has at least 91% sequence identity to SEQ ID NO:10. In embodiments, the protein has at least 92% sequence identity to SEQ ID NO:10. In embodiments, the protein has at least 93% sequence identity to SEQ ID NO:10. In embodiments, the protein has at least 94% sequence identity to SEQ ID NO:10. In embodiments, the protein has at least 95% sequence identity to SEQ ID NO:10. In embodiments, the protein has at least 96% sequence identity to SEQ ID NO:10. In embodiments, the protein has at least 97% sequence identity to SEQ ID NO:10. In embodiments, the protein has at least 98% sequence identity to SEQ ID NO:10. In embodiments, the protein has at least 99% sequence identity to SEQ ID NO:10. In embodiments, the protein has SEQ ID NO:10. In embodiments, the protein is SEQ ID NO:10. In embodiments, SEQ ID NO:10 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:12. In embodiments, the protein has at least 85% sequence identity to SEQ ID NO:12. In embodiments, the protein has at least 90% sequence identity to SEQ ID NO:12. In embodiments, the protein has at least 91% sequence identity to SEQ ID NO:12. In embodiments, the protein has at least 92% sequence identity to SEQ ID NO:12. In embodiments, the protein has at least 93% sequence identity to SEQ ID NO:12. In embodiments, the protein has at least 94% sequence identity to SEQ ID NO:12. In embodiments, the protein has at least 95% sequence identity to SEQ ID NO:12. In embodiments, the protein has at least 96% sequence identity to SEQ ID NO:12. In embodiments, the protein has at least 97% sequence identity to SEQ ID NO:12. In embodiments, the protein has at least 98% sequence identity to SEQ ID NO:12. In embodiments, the protein has at least 99% sequence identity to SEQ ID NO:12. In embodiments, the protein has SEQ ID NO:12. In embodiments, the protein is SEQ ID NO:12. In embodiments, SEQ ID NO:12 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:14. In embodiments, the protein has at least 85% sequence identity to SEQ ID NO:14. In embodiments, the protein has at least 90% sequence identity to SEQ ID NO:14. In embodiments, the protein has at least 91% sequence identity to SEQ ID NO:14. In embodiments, the protein has at least 92% sequence identity to SEQ ID NO:14. In embodiments, the protein has at least 93% sequence identity to SEQ ID NO:14. In embodiments, the protein has at least 94% sequence identity to SEQ ID NO:14. In embodiments, the protein has at least 95% sequence identity to SEQ ID NO:14. In embodiments, the protein has at least 96% sequence identity to SEQ ID NO:14. In embodiments, the protein has at least 97% sequence identity to SEQ ID NO:14. In embodiments, the protein has at least 98% sequence identity to SEQ ID NO:14. In embodiments, the protein has at least 99% sequence identity to SEQ ID NO:14. In embodiments, the protein has SEQ ID NO:14. In embodiments, the protein is SEQ ID NO:14. In embodiments, SEQ ID NO:14 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

In embodiments, the protein has at least 80% sequence identity to SEQ ID NO:16. In embodiments, the protein has at least 85% sequence identity to SEQ ID NO:16. In embodiments, the protein has at least 90% sequence identity to SEQ ID NO:16. In embodiments, the protein has at least 91% sequence identity to SEQ ID NO:16. In embodiments, the protein has at least 92% sequence identity to SEQ ID NO:16. In embodiments, the protein has at least 93% sequence identity to SEQ ID NO:16. In embodiments, the protein has at least 94% sequence identity to SEQ ID NO:16. In embodiments, the protein has at least 95% sequence identity to SEQ ID NO:16. In embodiments, the protein has at least 96% sequence identity to SEQ ID NO:16. In embodiments, the protein has at least 97% sequence identity to SEQ ID NO:16. In embodiments, the protein has at least 98% sequence identity to SEQ ID NO:16. In embodiments, the protein has at least 99% sequence identity to SEQ ID NO:16. In embodiments, the protein has SEQ ID NO:16. In embodiments, the protein is SEQ ID NO:16. In embodiments, SEQ ID NO:16 does not comprise an HA-tag having SEQ ID NO:31. Provided herein are nucleic acids encoding any one of the proteins described herein, including embodiments thereof. Provided herein are nucleic acids encoding any one of the proteins described herein, including embodiments thereof, and a human MIP3α protein. Provided herein are plasmids comprising nucleic acids encoding any one of the proteins described herein, including embodiments thereof. Provided herein are plasmids comprising nucleic acids encoding any one of the proteins described herein, including embodiments thereof, and a human MIP3α protein.

Provided herein are nucleic acids having at least 75% sequence identity, at least 80% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to any one of SEQ ID NO:1, SEQ ID NO:3, SEQ ID NO:5, SEQ ID NO:7, SEQ ID NO:9, SEQ ID NO:11, SEQ ID NO:13, or SEQ ID NO:15. In embodiments, the nucleic acid does not comprise an HA-tag having SEQ ID NO:30. In embodiments, the nucleic acids are referred to as plasmids.

In embodiments, the nucleic acid has at least 80% sequence identity to SEQ ID NO:1. In embodiments, the nucleic acid has at least 85% sequence identity to SEQ ID NO:1. In embodiments, the nucleic acid has at least 90% sequence identity to SEQ ID NO:1. In embodiments, the nucleic acid has at least 91% sequence identity to SEQ ID NO:1. In embodiments, the nucleic acid has at least 92% sequence identity to SEQ ID NO:1. In embodiments, the nucleic acid has at least 93% sequence identity to SEQ ID NO:1. In embodiments, the nucleic acid has at least 94% sequence identity to SEQ ID NO:1. In embodiments, the nucleic acid has at least 95% sequence identity to SEQ ID NO:1. In embodiments, the nucleic acid has at least 96% sequence identity to SEQ ID NO:1. In embodiments, the nucleic acid has at least 97% sequence identity to SEQ ID NO:1. In embodiments, the nucleic acid has at least 98% sequence identity to SEQ ID NO:1. In embodiments, the nucleic acid has at least 99% sequence identity to SEQ ID NO:1. In embodiments, the nucleic acid has SEQ ID NO:1. In embodiments, the nucleic acid is SEQ ID NO:1. In embodiments, SEQ ID NO:1 does not comprise an HA-tag having SEQ ID NO:30. In embodiments, the nucleic acid of SEQ ID NO:1 is referred to as a plasmid.

In embodiments, the nucleic acid has at least 80% sequence identity to SEQ ID NO:3. In embodiments, the nucleic acid has at least 85% sequence identity to SEQ ID NO:3. In embodiments, the nucleic acid has at least 90% sequence identity to SEQ ID NO:3. In embodiments, the nucleic acid has at least 91% sequence identity to SEQ ID NO:3. In embodiments, the nucleic acid has at least 92% sequence identity to SEQ ID NO:3. In embodiments, the nucleic acid has at least 93% sequence identity to SEQ ID NO:3. In embodiments, the nucleic acid has at least 94% sequence identity to SEQ ID NO:3. In embodiments, the nucleic acid has at least 95% sequence identity to SEQ ID NO:3. In embodiments, the nucleic acid has at least 96% sequence identity to SEQ ID NO:3. In embodiments, the nucleic acid has at least 97% sequence identity to SEQ ID NO:3. In embodiments, the nucleic acid has at least 98% sequence identity to SEQ ID NO:3. In embodiments, the nucleic acid has at least 99% sequence identity to SEQ ID NO:3. In embodiments, the nucleic acid has SEQ ID NO:3. In embodiments, the nucleic acid is SEQ ID NO:3. In embodiments, SEQ ID NO:3 does not comprise an HA-tag having SEQ ID NO:30. In embodiments, the nucleic acid of SEQ ID NO:3 is referred to as a plasmid.

In embodiments, the nucleic acid has at least 80% sequence identity to SEQ ID NO:5. In embodiments, the nucleic acid has at least 85% sequence identity to SEQ ID NO:5. In embodiments, the nucleic acid has at least 90% sequence identity to SEQ ID NO:5. In embodiments, the nucleic acid has at least 91% sequence identity to SEQ ID NO:5. In embodiments, the nucleic acid has at least 92% sequence identity to SEQ ID NO:5. In embodiments, the nucleic acid has at least 93% sequence identity to SEQ ID NO:5. In embodiments, the nucleic acid has at least 94% sequence identity to SEQ ID NO:5. In embodiments, the nucleic acid has at least 95% sequence identity to SEQ ID NO:5. In embodiments, the nucleic acid has at least 96% sequence identity to SEQ ID NO:5. In embodiments, the nucleic acid has at least 97% sequence identity to SEQ ID NO:5. In embodiments, the nucleic acid has at least 98% sequence identity to SEQ ID NO:5. In embodiments, the nucleic acid has at least 99% sequence identity to SEQ ID NO:5. In embodiments, the nucleic acid has SEQ ID NO:5. In embodiments, the nucleic acid is SEQ ID NO:5. In embodiments, SEQ ID NO:5 does not comprise an HA-tag having SEQ ID NO:30. In embodiments, the nucleic acid of SEQ ID NO:5 is referred to as a plasmid.

In embodiments, the nucleic acid has at least 80% sequence identity to SEQ ID NO:7. In embodiments, the nucleic acid has at least 85% sequence identity to SEQ ID NO:7. In embodiments, the nucleic acid has at least 90% sequence identity to SEQ ID NO:7. In embodiments, the nucleic acid has at least 91% sequence identity to SEQ ID NO:7. In embodiments, the nucleic acid has at least 92% sequence identity to SEQ ID NO:7. In embodiments, the nucleic acid has at least 93% sequence identity to SEQ ID NO:7. In embodiments, the nucleic acid has at least 94% sequence identity to SEQ ID NO:7. In embodiments, the nucleic acid has at least 95% sequence identity to SEQ ID NO:7. In embodiments, the nucleic acid has at least 96% sequence identity to SEQ ID NO:7. In embodiments, the nucleic acid has at least 97% sequence identity to SEQ ID NO:7. In embodiments, the nucleic acid has at least 98% sequence identity to SEQ ID NO:7. In embodiments, the nucleic acid has at least 99% sequence identity to SEQ ID NO:7. In embodiments, the nucleic acid has SEQ ID NO:7. In embodiments, the nucleic acid is SEQ ID NO:7. In embodiments, SEQ ID NO:7 does not comprise an HA-tag having SEQ ID NO:30. the nucleic acid of SEQ ID NO:7 is referred to as a plasmid.

In embodiments, the nucleic acid has at least 80% sequence identity to SEQ ID NO:9. In embodiments, the nucleic acid has at least 85% sequence identity to SEQ ID NO:9. In embodiments, the nucleic acid has at least 90% sequence identity to SEQ ID NO:9. In embodiments, the nucleic acid has at least 91% sequence identity to SEQ ID NO:9. In embodiments, the nucleic acid has at least 92% sequence identity to SEQ ID NO:9. In embodiments, the nucleic acid has at least 93% sequence identity to SEQ ID NO:9. In embodiments, the nucleic acid has at least 94% sequence identity to SEQ ID NO:9. In embodiments, the nucleic acid has at least 95% sequence identity to SEQ ID NO:9. In embodiments, the nucleic acid has at least 96% sequence identity to SEQ ID NO:9. In embodiments, the nucleic acid has at least 97% sequence identity to SEQ ID NO:9. In embodiments, the nucleic acid has at least 98% sequence identity to SEQ ID NO:9. In embodiments, the nucleic acid has at least 99% sequence identity to SEQ ID NO:9. In embodiments, the nucleic acid has SEQ ID NO:9. In embodiments, the nucleic acid is SEQ ID NO:9. In embodiments, SEQ ID NO:9 does not comprise an HA-tag having SEQ ID NO:30. the nucleic acid of SEQ ID NO:9 is referred to as a plasmid.

In embodiments, the nucleic acid has at least 80% sequence identity to SEQ ID NO:11. In embodiments, the nucleic acid has at least 85% sequence identity to SEQ ID NO:11. In embodiments, the nucleic acid has at least 90% sequence identity to SEQ ID NO:11. In embodiments, the nucleic acid has at least 91% sequence identity to SEQ ID NO:11. In embodiments, the nucleic acid has at least 92% sequence identity to SEQ ID NO:11. In embodiments, the nucleic acid has at least 93% sequence identity to SEQ ID NO:11. In embodiments, the nucleic acid has at least 94% sequence identity to SEQ ID NO:11. In embodiments, the nucleic acid has at least 95% sequence identity to SEQ ID NO:11. In embodiments, the nucleic acid has at least 96% sequence identity to SEQ ID NO:11. In embodiments, the nucleic acid has at least 97% sequence identity to SEQ ID NO:11. In embodiments, the nucleic acid has at least 98% sequence identity to SEQ ID NO:11. In embodiments, the nucleic acid has at least 99% sequence identity to SEQ ID NO:11. In embodiments, the nucleic acid has SEQ ID NO:11. In embodiments, the nucleic acid is SEQ ID NO:11. In embodiments, SEQ ID NO:11 does not comprise an HA-tag having SEQ ID NO:30. the nucleic acid of SEQ ID NO:11 is referred to as a plasmid.

In embodiments, the nucleic acid has at least 80% sequence identity to SEQ ID NO:13. In embodiments, the nucleic acid has at least 85% sequence identity to SEQ ID NO:13. In embodiments, the nucleic acid has at least 90% sequence identity to SEQ ID NO:13. In embodiments, the nucleic acid has at least 91% sequence identity to SEQ ID NO:13. In embodiments, the nucleic acid has at least 92% sequence identity to SEQ ID NO:13. In embodiments, the nucleic acid has at least 93% sequence identity to SEQ ID NO:13. In embodiments, the nucleic acid has at least 94% sequence identity to SEQ ID NO:13. In embodiments, the nucleic acid has at least 95% sequence identity to SEQ ID NO:13. In embodiments, the nucleic acid has at least 96% sequence identity to SEQ ID NO:13. In embodiments, the nucleic acid has at least 97% sequence identity to SEQ ID NO:13. In embodiments, the nucleic acid has at least 98% sequence identity to SEQ ID NO:13. In embodiments, the nucleic acid has at least 99% sequence identity to SEQ ID NO:13. In embodiments, the nucleic acid has SEQ ID NO:13. In embodiments, the nucleic acid is SEQ ID NO:13. In embodiments, SEQ ID NO:13 does not comprise an HA-tag having SEQ ID NO:30. the nucleic acid of SEQ ID NO:13 is referred to as a plasmid.

In embodiments, the nucleic acid has at least 80% sequence identity to SEQ ID NO:15. In embodiments, the nucleic acid has at least 85% sequence identity to SEQ ID NO:15. In embodiments, the nucleic acid has at least 90% sequence identity to SEQ ID NO:15. In embodiments, the nucleic acid has at least 91% sequence identity to SEQ ID NO:15. In embodiments, the nucleic acid has at least 92% sequence identity to SEQ ID NO:15. In embodiments, the nucleic acid has at least 93% sequence identity to SEQ ID NO:15. In embodiments, the nucleic acid has at least 94% sequence identity to SEQ ID NO:15. In embodiments, the nucleic acid has at least 95% sequence identity to SEQ ID NO:15. In embodiments, the nucleic acid has at least 96% sequence identity to SEQ ID NO:15. In embodiments, the nucleic acid has at least 97% sequence identity to SEQ ID NO:15. In embodiments, the nucleic acid has at least 98% sequence identity to SEQ ID NO:15. In embodiments, the nucleic acid has at least 99% sequence identity to SEQ ID NO:15. In embodiments, the nucleic acid has SEQ ID NO:15. In embodiments, the nucleic acid is SEQ ID NO:7. In embodiments, SEQ ID NO:15 does not comprise an HA-tag having SEQ ID NO:30. the nucleic acid of SEQ ID NO:15 is referred to as a plasmid.

Description of the pSARS-CoV-2-FL construct (SEQ ID NO:1): the human MIP3α secretion leader sequence (SL), without human MIP3α gene, was cloned in-frame with SARS-CoV-2-S gene with the transmembrane (TM) and all the amino acids deleted from the cytoplasmic domain (CT), i.e., the deletion of SEQ ID NO:42. The protein encoded by this construct is set forth as SEQ ID NO:2.

Description of the pMIP3α-SARS-CoV-2-FL construct (SEQ ID NO:3): the human MIP3α, comprising the secretion leader sequence (SL), was cloned in-frame with SARS-CoV-2-S gene with the transmembrane (TM) and all the amino acids deleted from the cytoplasmic domain (CT), i.e., the deletion of SEQ ID NO:42. The protein encoded by this construct is set forth as SEQ ID NO:4.

Description of the pSARS-CoV-2-ΔT construct (SEQ ID NO:5): the human MIP3α secretion leader sequence (SL), without human MIP3α gene, was cloned in-frame with SARS-CoV-2-S gene without the transmembrane (TM) and cytoplasmic domain (CT). The protein encoded by this construct is set forth as SEQ ID NO:6.

Description of the pMIP3α-SARS-CoV-2-ΔT construct (SEQ ID NO:7): the human MIP3α, comprising the secretion leader sequence (SL), was cloned in-frame with SARS-CoV-2-S gene without the transmembrane (TM) and cytoplasmic domain (CT). The protein encoded by this construct is set forth as SEQ ID NO:8.

Description of the pSARS-CoV-2-ΔTS construct (SEQ ID NO:9): the human MIP3α secretion leader sequence (SL), was cloned in-frame with genetically modified SARS-CoV-2-S gene, in which the two protease cleavage sites were removed (ΔS1/S2: removed RRAR amino acids and ΔS2′: removed PSKR (SEQ ID NO:21) amino acids). The protein encoded by this construct is set forth as SEQ ID NO:10.

Description of the pMIP3α-SARS-CoV-2-ΔTS construct (SEQ ID NO:11): the human MIP3α, comprising the secretion leader sequence (SL), was cloned in-frame with genetically modified SARS-CoV-2-S gene, in which the two protease cleavage sites were removed (ΔS1/S2: removed RRAR (SEQ ID NO:20) amino acids and ΔS2′: removed PSKR amino acids). The protein encoded by this construct is set forth as SEQ ID NO:12.

Description of the pSARS-CoV-2-ΔTSL construct (SEQ ID NO:13): the human MIP3α secretion leader sequence (SL), was cloned in-frame with a genetically modified SARS-CoV-2-S gene, in which the two protease cleavage sites (ΔS1/S2 and ΔS2′) were removed and replaced with flexible linker (G₄S)₃ inserts. The protein encoded by this construct is set forth as SEQ ID NO:14.

Description of the pMIP3α-SARS-CoV-2-ΔTSL construct (SEQ ID NO:15): the human MIP3α, comprising the secretion leader sequence (SL), was cloned in-frame with a genetically modified SARS-CoV-2-S gene, in which the two protease cleavage sites (ΔS1/S2 and ΔS2′) were removed and replaced with flexible linker (G₄S)₃ inserts. The protein encoded by this construct is set forth as SEQ ID NO:16.

All the constructs described above were cloned in the pUMVC3 vector, described in Example 10.

In another aspect is provided a pharmaceutical composition comprising a protein as provided herein and a pharmaceutically acceptable excipient. In another aspect is provided a pharmaceutical composition comprising a nucleic acid as provided herein and a pharmaceutically acceptable excipient. In another aspect is provided a pharmaceutical composition comprising a plasmid as provided herein and a pharmaceutically acceptable excipient.

In embodiments, the pharmaceutical composition is a vaccine composition and the pharmaceutically acceptable excipient is a vaccine adjuvant. Thus, provided herein is a vaccine comprising a nucleic acid as provided herein and an adjuvant. In embodiments, the disclosure provides a vaccine comprising a nucleic acid of SEQ ID NO:1, including any sequence identities and embodiments described herein, and an adjuvant. In embodiments, the disclosure provides a vaccine comprising a nucleic acid of SEQ ID NO:3, including any sequence identities and embodiments described herein, and an adjuvant. In embodiments, the disclosure provides a vaccine comprising a nucleic acid of SEQ ID NO:5, including any sequence identities and embodiments described herein, and an adjuvant. In embodiments, the disclosure provides a vaccine comprising a nucleic acid of SEQ ID NO:6, including any sequence identities and embodiments described herein, and an adjuvant. In embodiments, the disclosure provides a vaccine comprising a nucleic acid of SEQ ID NO:7, including any sequence identities and embodiments described herein, and an adjuvant. In embodiments, the disclosure provides a vaccine comprising a nucleic acid of SEQ ID NO:11, including any sequence identities and embodiments described herein, and an adjuvant. In embodiments, the disclosure provides a vaccine comprising a nucleic acid of SEQ ID NO:13, including any sequence identities and embodiments described herein, and an adjuvant. In embodiments, the disclosure provides a vaccine comprising a nucleic acid of SEQ ID NO:15, including any sequence identities and embodiments described herein, and an adjuvant.

In embodiments, the disclosure provides a vaccine comprising a nucleic acid encoding a protein having SEQ ID NO:2, including any sequence identities and embodiments described herein, and an adjuvant. In embodiments, the disclosure provides a vaccine comprising a nucleic acid encoding a protein having SEQ ID NO:4, including any sequence identities and embodiments described herein, and an adjuvant. In embodiments, the disclosure provides a vaccine comprising a nucleic acid encoding a protein having SEQ ID NO:6, including any sequence identities and embodiments described herein, and an adjuvant. In embodiments, the disclosure provides a vaccine comprising a nucleic acid encoding a protein having SEQ ID NO:8, including any sequence identities and embodiments described herein, and an adjuvant. In embodiments, the disclosure provides a vaccine comprising a nucleic acid encoding a protein having SEQ ID NO:10, including any sequence identities and embodiments described herein, and an adjuvant. In embodiments, the disclosure provides a vaccine comprising a nucleic acid encoding a protein having SEQ ID NO:12, including any sequence identities and embodiments described herein, and an adjuvant. In embodiments, the disclosure provides a vaccine comprising a nucleic acid encoding a protein having SEQ ID NO:14, including any sequence identities and embodiments described herein, and an adjuvant. In embodiments, the disclosure provides a vaccine comprising a nucleic acid encoding a protein having SEQ ID NO:16, including any sequence identities and embodiments described herein, and an adjuvant.

In embodiments, the disclosure provides methods of increasing immunity to a SARS coronavirus in a subject in need thereof comprising administering to the subject an effective amount of a protein as described herein, including embodiments thereof. In embodiments, the disclosure provides methods of increasing immunity to a SARS coronavirus in a subject in need thereof comprising administering to the subject an effective amount of a nucleic acid as described herein, including embodiments thereof. In embodiments, the disclosure provides methods of increasing immunity to a SARS coronavirus in a subject in need thereof comprising administering to the subject an effective amount of a plasmid as described herein, including embodiments thereof. In embodiments, the disclosure provides methods of increasing immunity to a SARS coronavirus in a subject in need thereof comprising administering to the subject an effective amount of a pharmaceutical composition as described herein, including embodiments thereof. In embodiments, the disclosure provides methods of increasing immunity to a SARS coronavirus in a subject in need thereof comprising administering to the subject an effective amount of a vaccine as described herein, including embodiments thereof. In embodiments, the SARS coronavirus is SARS-CoV-1. In embodiments, the SARS coronavirus is SARS-CoV-2. In embodiments, the subject is a human.

In embodiments, the disclosure provides methods of providing acquired immunity to a SARS coronavirus in a subject in need thereof comprising administering to the subject an effective amount of a protein as described herein, including embodiments thereof. In embodiments, the disclosure provides methods of providing acquired immunity to a SARS coronavirus in a subject in need thereof comprising administering to the subject an effective amount of a nucleic acid as described herein, including embodiments thereof. In embodiments, the disclosure provides methods of providing acquired immunity to a SARS coronavirus in a subject in need thereof comprising administering to the subject an effective amount of a plasmid as described herein, including embodiments thereof. In embodiments, the disclosure provides methods of providing acquired immunity to a SARS coronavirus in a subject in need thereof comprising administering to the subject an effective amount of a pharmaceutical composition as described herein, including embodiments thereof, and a pharmaceutically acceptable excipient. In embodiments, the disclosure provides methods of providing acquired immunity to a SARS coronavirus in a subject in need thereof comprising administering to the subject an effective amount of a vaccine as described herein, including embodiments thereof, and an adjuvant. In embodiments, the SARS coronavirus is SARS-CoV-1. In embodiments, the SARS coronavirus is SARS-CoV-2. In embodiments, the subject is a human.

In embodiments, the disclosure provides methods of preventing COVID-19 in a subject in need thereof comprising administering to the subject an effective amount of a protein as described herein, including embodiments thereof. In embodiments, the disclosure provides methods of preventing COVID-19 in a subject in need thereof comprising administering to the subject an effective amount of a nucleic acid as described herein, including embodiments thereof. In embodiments, the disclosure provides methods of preventing COVID-19 in a subject in need thereof comprising administering to the subject an effective amount of a pharmaceutical composition comprising a protein as described herein, including embodiments thereof, and a pharmaceutically acceptable excipient. In embodiments, the disclosure provides methods of preventing COVID-19 in a subject in need thereof comprising administering to the subject an effective amount of a pharmaceutical composition as described herein, including embodiments thereof. In embodiments, the disclosure provides methods of preventing COVID-19 in a subject in need thereof comprising administering to the subject an effective amount of a vaccine as described herein, including embodiments thereof. In embodiments, the subject is a human.

In embodiments, the disclosure provides methods of reducing the incidence of hospitalization or death from COVID-19 in a subject in need thereof comprising administering to the subject an effective amount of a protein as described herein, including embodiments thereof. In embodiments, the disclosure provides methods of reducing the incidence of hospitalization or death from COVID-19 in a subject in need thereof comprising administering to the subject an effective amount of a nucleic acid as described herein, including embodiments thereof. In embodiments, the disclosure provides methods of reducing the incidence of hospitalization or death from COVID-19 in a subject in need thereof comprising administering to the subject an effective amount of a plasmid as described herein, including embodiments thereof, and a pharmaceutically acceptable excipient. In embodiments, the disclosure provides methods of reducing the incidence of hospitalization or death from COVID-19 in a subject in need thereof comprising administering to the subject an effective amount of a pharmaceutical composition as described herein, including embodiments thereof. In embodiments, the disclosure provides methods of reducing the incidence of hospitalization or death from COVID-19 in a subject in need thereof comprising administering to the subject an effective amount of a vaccine as described herein, including embodiments thereof. In embodiments, the methods are for reducing the incidence of hospitalization. In embodiments, the methods are for reducing the incidence of death. In embodiments, the subject is a human.

In embodiments, the proteins, nucleic acids, pharmaceutical compositions, or vaccines are administered by parenteral injection. In embodiments, the proteins, nucleic acids, pharmaceutical compositions, or vaccines are administered by intramuscular injection, subcutaneous injection, or pulmonary administration. In embodiments, the proteins, nucleic acids, pharmaceutical compositions, or vaccines are administered by intramuscular injection. In embodiments, the proteins, nucleic acids, pharmaceutical compositions, or vaccines are administered by subcutaneous injection. In embodiments, the proteins, nucleic acids, pharmaceutical compositions, or vaccines are administered by pulmonary administration. In embodiments, the proteins, nucleic acids, pharmaceutical compositions, or vaccines are administered intranasally. In embodiments, the proteins, nucleic acids, pharmaceutical compositions, or vaccines are administered orally.

In embodiments, the pharmaceutical composition is formulated as an intradermal injection or as an intramuscular injection. In embodiments, the pharmaceutical composition is formulated as an intradermal injection. In embodiments, the pharmaceutical composition is formulated as an intradermal injection. In embodiments, the pharmaceutical composition is formulated as a subcutaneous injection. In embodiments, the vaccine is formulated as an intradermal injection or as an intramuscular injection. In embodiments, the vaccine is formulated as an intradermal injection. In embodiments, the vaccine is formulated as a subcutaneous injection. In embodiments, the vaccine is formulated as an intramuscular injection.

In embodiments, the pharmaceutical composition is present in a needle free device. In embodiments, the vaccine is present in a needle-free device. In embodiments, the needle free device is PharmaJet® (by PharmaJet, Golden, Colorado). In embodiments, the disclosure provides a device comprising a vaccine as described herein, including all embodiments thereof. In embodiments, the disclosure provides a syringe comprising a vaccine as described herein, including all embodiments thereof. In embodiments, the disclosure provides a needle-free device comprising a vaccine as described herein, including all embodiments thereof. In embodiments, the disclosure provides an injector comprising a vaccine as described herein, including all embodiments thereof. In embodiments, the disclosure provides an injector comprising a vaccine as described herein, including all embodiments thereof, where the injector is a needle-free injector.

In embodiments, the amount of protein, nucleic acid, or plasmid used in the pharmaceutical compositions or vaccines described herein for the methods described herein is from 500 μg to 2500 μg. In embodiments, the amount of protein, nucleic acid, or plasmid used in the pharmaceutical compositions or vaccines described herein for the methods described herein is from about 500 μg to about 2,500 μg. In embodiments, the amount of protein, nucleic acid, or plasmid used in the pharmaceutical compositions or vaccines described herein for the methods described herein is from about 500 μg to about 2,000 μg. In embodiments, the amount of protein, nucleic acid, or plasmid used in the pharmaceutical compositions or vaccines described herein for the methods described herein is from about 500 μg to about 1,500 μg. In embodiments, the amount of protein, nucleic acid, or plasmid used in the pharmaceutical compositions or vaccines described herein for the methods described herein is from about 500 μg to about 1,000 μg. In embodiments, the amount of protein, nucleic acid, or plasmid used in the pharmaceutical compositions or vaccines described herein for the methods described herein is from about 1 μg to about 4,000 μg. In embodiments, the amount of protein, nucleic acid, or plasmid used in the pharmaceutical compositions or vaccines described herein for the methods described herein is from about 1 μg to about 3,000 μg. In embodiments, the amount of protein, nucleic acid, or plasmid used in the pharmaceutical compositions or vaccines described herein for the methods described herein is from about 1 μg to about 2,500 μg. In embodiments, the amount of protein, nucleic acid, or plasmid used in the pharmaceutical compositions or vaccines described herein for the methods described herein is from about 1 μg to about 2,000 μg. In embodiments, the amount of protein, nucleic acid, or plasmid used in the pharmaceutical compositions or vaccines described herein for the methods described herein is from about 100 μg to about 2,500 μg. In embodiments, the amount of protein, nucleic acid, or plasmid used in the pharmaceutical compositions or vaccines described herein for the methods described herein is from about 100 μg to about 2,000 μg. In embodiments, the amount of protein, nucleic acid, or plasmid used in the pharmaceutical compositions or vaccines described herein for the methods described herein is from about 100 μg to about 1,500 μg. In embodiments, the amount of protein, nucleic acid, or plasmid used in the pharmaceutical compositions or vaccines described herein for the methods described herein is from about 100 μg to about 1,000 μg. In embodiments, the amount of protein, nucleic acid, or plasmid used in the pharmaceutical compositions or vaccines described herein for the methods described herein is from about 250 μg to about 2,500 μg. In embodiments, the amount of protein, nucleic acid, or plasmid used in the pharmaceutical compositions or vaccines described herein for the methods described herein is from about 250 μg to about 2,000 μg. In embodiments, the amount of protein, nucleic acid, or plasmid used in the pharmaceutical compositions or vaccines described herein for the methods described herein is from about 250 μg to about 1,500 μg. In embodiments, the amount of protein, nucleic acid, or plasmid used in the pharmaceutical compositions or vaccines described herein for the methods described herein is from about 250 μg to about 1,000 μg.

In embodiments, the disclosure provide a kit comprising a vaccine as described herein, including embodiments thereof, a needle-free injector, and instructions for use. In embodiments, the disclosure provide a kit comprising a vaccine as described herein, including embodiments thereof, a syringe, and instructions for use. In embodiments, the disclosure provides a kit comprising an injector as described herein, including embodiments thereof, a syringe, and instructions for use. In embodiments, the disclosure provides a kit comprising a syringe as described herein, including embodiments thereof, a syringe, and instructions for use. In embodiments, the disclosure provides a vial of as described herein, including embodiments thereof, a syringe, and instructions for use. The instructions for use are instructions for use in any of the methods described herein and/or instructions for administering the vaccine to a subject.

### Embodiments P1-P21

Embodiment P1. A SARS-CoV-2 S protein comprising a truncated cytoplasmic domain.

Embodiment P2. The SARS-CoV-2 S protein of Embodiment P1 further comprising a truncated transmembrane domain.

Embodiment P3. The SARS-CoV-2 S protein of Embodiment P2 wherein the SARS-CoV-2 S protein does not comprise a S1/S2 protease cleavage site and/or a S2′ protease cleavage site.

Embodiment P4. The SARS-CoV-2 S protein of Embodiment P3 further where said S1/S2 protease cleavage site, and/or S2′ protease cleavage site is replaced with a peptide linker.

Embodiment P5. The SARS-CoV-2 S protein of Embodiment P4 where said peptide linker is a (G₄S)₃ linker.

Embodiment P6. A nucleic acid encoding the SARS-CoV-2 S protein of one of Embodiments P1 to P5.

Embodiment P7. The nucleic acid of Embodiment P7 further encoding a human MIP3α protein.

Embodiment P8. A nucleic acid encoding SARS-CoV-2 S protein and a human MIP3α protein.

Embodiment P9. The nucleic acid of any of Embodiments P6-P8 wherein said nucleic acid is a plasmid.

Embodiment P10. The plasmid of Embodiment P9, wherein the expression of the SARS-CoV-2 S protein is driven by a ubiquitous promoter.

Embodiment P11. The plasmid of Embodiment P10, wherein the ubiquitous promoter is a CMV promoter.

Embodiment P12. The plasmid of Embodiment P11, wherein a polyadenylation signal is located downstream of the sequence encoding the SARS-CoV-2 S protein.

Embodiment P13. The plasmid of Embodiment P12, wherein the polyadenylation signal is a rabbit β-globulin polyadenylation signal.

Embodiment P14. A pharmaceutical composition comprising the nucleic acid of one of Embodiments P1 to P13 and a pharmaceutically acceptable excipient.

Embodiment P15. The pharmaceutical composition of Embodiment P14, wherein said pharmaceutical composition is a vaccine composition and said pharmaceutically acceptable excipient is a vaccine adjuvant.

Embodiment P16. The pharmaceutical composition of Embodiment P15, wherein said pharmaceutical composition is formulated as an intradermal injection.

Embodiment P17. The pharmaceutical composition of Embodiment P16, wherein said pharmaceutical composition is present in a needle free device.

Embodiment P18. The pharmaceutical composition of Embodiment P17, wherein the amount of nucleic acid is 500-2500 μg

Embodiment P19. A method of increasing immunity to SARS-CoV-2 virus to a human subject in need thereof, the method comprising administering an effective amount of the nucleic acid of one of Embodiments P6-P13 or the pharmaceutical composition of one of Embodiments P14-P18.

Embodiment P20. A method of providing acquired immunity to SARS-CoV-2 virus to a human subject in need thereof, the method comprising administering an effective amount of the nucleic acid of one of Embodiments P6-P13 or the pharmaceutical composition of one of Embodiments P14-P18.

Embodiment P21. A method of preventing COVID-19 in a human subject in need thereof, the method comprising administering an effective amount of the nucleic acid of one of Embodiments P6-P13 or the pharmaceutical composition of one of Embodiments P14-P18.

### Embodiments 1 to 60

Embodiment 1. A protein having at least 85% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

Embodiment 2. The protein of Embodiment 1 having at least 90% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

Embodiment 3. The protein of Embodiment 1 having at least 95% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

Embodiment 4. The protein of Embodiment 1 having SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

Embodiment 5. The protein of any one of Embodiments 1 to 4, wherein the protein does not comprise an HA-tag having SEQ ID NO:31.

Embodiment 6. A nucleic acid encoding the protein of any one of Embodiments 1 to 5.

Embodiment 7. A nucleic acid having at least 85% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

Embodiment 8. The nucleic acid of Embodiment 6, having at least 90% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

Embodiment 9. The nucleic acid of Embodiment 6, having at least 95% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

Embodiment 10. The nucleic acid of any one of Embodiments 7 to 9, wherein the nucleic acid does not comprise an HA-tag having SEQ ID NO:30.

Embodiment 11. A protein comprising an amino acid sequence of formula (I): R¹-L¹-R²-L²-R³-R⁴-R⁵, wherein: R¹ is an amino acid sequence having at least 85% sequence identity to SEQ ID NO:46; L¹ is SEQ ID NO:20, absent, —S(G₄S)ₓ—, -(G₄S)ₓ—, —S(G₃S)ₓ—, or -(G₃S)ₓ—; wherein x is an integer from 1 to 10; R² is an amino acid sequence having at least 85% sequence identity to SEQ ID NO:47; L² is SEQ ID NO:21, absent, —S(G₄S)ₓ—, -(G₄S)ₓ—, —S(G₃S)ₓ—, or -(G₃S)ₓ—; wherein x is an integer from 1 to 10; R³ is an amino acid sequence having at least 85% sequence identity to SEQ ID NO:48; R⁴ is absent, an amino acid sequence having at least 85% sequence identity to SEQ ID NO:41, or a truncated transmembrane domain; and R³ is absent or a truncated cytoplasmic domain; provided that R⁵ is absent when R⁴ is absent or a truncated transmembrane domain.

Embodiment 12. The protein of Embodiment 11, wherein R¹ is an amino acid sequence having at least 90% sequence identity to SEQ ID NO:46; R² is an amino acid sequence having at least 90% sequence identity to SEQ ID NO:47; R³ is an amino acid sequence having at least 90% sequence identity to SEQ ID NO:48.

Embodiment 13. The protein of Embodiment 12, wherein R¹ is an amino acid sequence having at least 95% sequence identity to SEQ ID NO:46; R² is an amino acid sequence having at least 95% sequence identity to SEQ ID NO:47; and R³ is an amino acid sequence having at least 95% sequence identity to SEQ ID NO:48.

Embodiment 14. The protein of Embodiment 13, wherein R¹ is an amino acid sequence having SEQ ID NO:46; R² is an amino acid sequence having SEQ ID NO:47; and R³ is an amino acid sequence having SEQ ID NO:48.

Embodiment 15. The protein of any one of Embodiments 11 to 14, wherein R⁴ is an amino acid sequence having at least 90% sequence identity to SEQ ID NO:41.

Embodiment 16. The protein of Embodiment 15, wherein R⁴ is an amino acid sequence having at least 95% sequence identity to SEQ ID NO:41.

Embodiment 17. The protein of Embodiment 16, wherein R⁴ is an amino acid sequence having SEQ ID NO:41.

Embodiment 18. The protein of any one of Embodiment 11 to 17, wherein R⁵ is absent.

Embodiment 19. The protein of any one of Embodiment 11 to 17, wherein R³ is a truncated cytoplasmic domain.

Embodiment 20. The protein of any one of Embodiments 11 to 16, wherein R⁴ is absent and R³ is absent.

Embodiment 21. The protein of any one of Embodiments 11 to 16, wherein R⁴ is a truncated transmembrane domain and R³ is absent.

Embodiment 22. The protein of any one of Embodiments 11 to 21, wherein L¹ is absent, —S(G₄S)ₓ—, -(G₄S)ₓ—, —S(G₃S)ₓ—, or -(G₃S)ₓ—; wherein x is an integer from 1 to 10; and L² is absent, —S(G₄S)ₓ—, -(G₄S)ₓ—, —S(G₃S)ₓ—, or -(G₃S)ₓ—; wherein x is an integer from 1 to 10.

Embodiment 23. The protein of Embodiment 22, wherein L¹ is absent and L² is absent.

Embodiment 24. The protein of Embodiment 22, wherein L¹ is —S(G₄S)ₓ—, -(G₄S)ₓ—, —S(G₃S)ₓ—, or -(G₃S)ₓ—; wherein x is an integer from 1 to 6; and L² is —S(G₄S)ₓ—, -(G₄S)ₓ—, —S(G₃S)ₓ—, or -(G₃S)ₓ—; wherein x is an integer from 1 to 6.

Embodiment 25. The protein of Embodiment 24, wherein L¹ is -(G₄S)ₓ— or -(G₃S)ₓ—; where x is an integer from 1 to 3; and L² is -(G₄S)ₓ— or -(G₃S)ₓ—; where x is an integer from 1 to 3.

Embodiment 26. The protein of any one of Embodiments 11 to 25, further comprising an amino acid sequence of a human MIP3α protein.

Embodiment 27. The protein of Embodiment 26, wherein the human MIP3α protein has the amino acid sequence of SEQ ID NO:29.

Embodiment 28. The protein of Embodiment 26 or 27, wherein the human MIP3α protein is located at position on the N-terminus of R¹.

Embodiment 29. A nucleic acid encoding the protein of any one of Embodiments 11 to 28.

Embodiment 30. A SARS-CoV-2 spike (S) protein comprising a truncated cytoplasmic domain.

Embodiment 31. A SARS-CoV-2 spike (S) protein comprising a truncated transmembrane domain; wherein the protein does not comprise the amino acid sequence of SEQ ID NO:42.

Embodiment 32. The SARS-CoV-2 spike (S) protein of Embodiment 30 or 31, wherein the SARS-CoV-2 spike (S) protein has at least 85% sequence identity to SEQ ID NO:4 or has at least 85% sequence identity to SEQ ID NO:45.

Embodiment 33. A SARS-CoV-2 spike (S) protein having at least 85% sequence identity to SEQ ID NO:4 or has at least 85% sequence identity to SEQ ID NO:45; provided that SEQ ID NO:44 and SEQ ID NO:45 do not comprise the amino acid sequence of SEQ ID NO:40.

Embodiment 34. The SARS-CoV-2 spike (S) protein of any one of Embodiments 30 to 33, wherein the S1/S2 protease cleavage site in the SARS-CoV-2 spike (S) protein is absent or replaced with a peptide linker.

Embodiment 35. The SARS-CoV-2 spike (S) protein of any one of Embodiments 30 to 34, wherein the S2′ protease cleavage site in the SARS-CoV-2 spike (S) protein is absent or replaced with a peptide linker.

Embodiment 36. The SARS-CoV-2 spike (S) protein of Embodiment 35, wherein the S1/S2 protease cleavage site is replaced with a peptide linker and the S2′ protease cleavage site is replaced with a peptide linker.

Embodiment 37. The SARS-CoV-2 spike (S) protein of Embodiment 36, wherein the peptide linker is —S(G₄S)ₓ—, -(G₄S)ₓ—, —S(G₃S)ₓ—, or -(G₃S)ₓ—; wherein x is an integer from 1 to 6.

Embodiment 38. The SARS-CoV-2 spike (S) protein of Embodiment 37, where the peptide linker is -(G₄S)₃—.

Embodiment 39. The SARS-CoV-2 spike (S) protein of any one of Embodiments 34 to 38, wherein the S1/S2 protease cleavage site in the SARS-CoV-2 spike (S) protein is absent and the S2′ protease cleavage site in the SARS-CoV-2 spike (S) protein is absent.

Embodiment 40. The SARS-CoV-2 spike (S) protein of any one of Embodiments 30 to 39, further comprising a human MIP3α protein.

Embodiment 41. A nucleic acid encoding the SARS-CoV-2 spike (S) protein of any one of Embodiments 30 to 41.

Embodiment 42. A plasmid comprising the nucleic acid of any one of Embodiments 6-10, 29, and 41.

Embodiment 43. The plasmid of Embodiment 42, further comprising a nucleic acid encoding a human MIP3α protein.

Embodiment 44. The plasmid of Embodiment 42 or 43, further comprising a ubiquitous promoter.

Embodiment 45. The plasmid of Embodiment 44, wherein the ubiquitous promoter is a CMV promoter.

Embodiment 46. The plasmid of any one of Embodiments 42 to 45, further comprising a polyadenylation signal located downstream of the nucleic acid encoding the protein.

Embodiment 47. The plasmid of Embodiment 46, wherein the polyadenylation signal is a rabbit β-globulin polyadenylation signal.

Embodiment 48. A pharmaceutical composition comprising: (i) the protein of any one of Embodiments 1-5, 11-28, and 30-40, and a pharmaceutically acceptable excipient, (ii) the nucleic acid of one of Embodiments 6-10, 29, and 41, and a pharmaceutically acceptable excipient; or (iii) the plasmid of any one of Embodiments 42-47.

Embodiment 49. A vaccine comprising: (i) the nucleic acid of one of Embodiments 6-10, 29, and 41 and an adjuvant, or (ii) the plasmid of any one of Embodiments 42-47.

Embodiment 50. A method of increasing immunity to a SARS coronavirus in a human in need thereof, the method comprising administering to the human an effective amount of: (i) the protein of any one of Embodiments 1-5, 11-28, and 30-40; (ii) the nucleic acid of one of Embodiments 6-10, 29, and 41; (iii) the plasmid of any one of Embodiments 42-47; (iv) the pharmaceutical composition of Embodiment 48; or (v) the vaccine of Embodiment 49.

Embodiment 51. A method of providing acquired immunity to a SARS coronavirus in a human in need thereof, the method comprising administering to the human an effective amount of: (i) the protein of any one of Embodiments 1-5, 11-28, and 30-40; (ii) the nucleic acid of one of Embodiments 6-10, 29, and 41; (iii) the plasmid of any one of Embodiments 42-47; (iv) the pharmaceutical composition of Embodiment 48; or (v) the vaccine of Embodiment 49.

Embodiment 52. The method of Embodiment 50 or 51, wherein the SARS coronavirus is SARS-CoV-2.

Embodiment 53. A method of preventing COVID-19 in a human in need thereof, the method comprising administering to the human an effective amount of: (i) the protein of any one of Embodiments 1-5, 11-28, and 30-40; (ii) the nucleic acid of one of Embodiments 6-10, 29, and 41; (iii) the plasmid of any one of Embodiments 42-47; (iv) the pharmaceutical composition of Embodiment 48; or (v) the vaccine of Embodiment 49.

Embodiment 54. The method of any one of Embodiments 50 to 53, comprising administering the effective amount of the nucleic acid via intramuscular injection, subcutaneous injection, or pulmonary administration.

Embodiment 55. The method of any one of Embodiments 50 to 54, comprising administering the effective amount of the nucleic acid via a needle-free injector.

Embodiment 56. An injector comprising the vaccine of Embodiment 49.

Embodiment 57. The injector of Embodiment 56, wherein the injector is a needle-free injector.

Embodiment 58. A syringe comprising the vaccine of Embodiment 49.

Embodiment 59. A vial comprising the vaccine of Embodiment 49.

Embodiment 60. A kit comprising: (a) the vaccine of Embodiment 49, a needle-free injector, and instructions for use; (b) the vaccine of Embodiment 49, a syringe, and instructions for use; (c) the injector of Embodiment 56 or 57 and instructions for use; (d) the syringe of Embodiment 58 and instructions for use; or (e) the vial of Embodiment 59 and instructions for use.

### EXAMPLES

### Example 1

Novel Chemokine-Antigen Fusion DNA Vaccine Design: Published Preclinical Studies

In contrast to expressing proteins, DNA vaccines are relatively simple and easy to produce. In vivo expression of foreign genes encoding the tumor antigen by DNA vaccination requires only that the gene is cloned into an expression cassette under eukaryotic or viral regulatory element control and delivered in solution intramuscularly or intradermally. The effectiveness of vaccines against infectious pathogens and tumors depends in large part on the efficient delivery of the relevant antigens to antigen-presenting cells (APC), particularly dendritic cells (DC), for processing into immunogenic peptides and presentation to T cells. Targeting antigens to APC via ligands for cell-surface receptors, which enable internalization and processing of the antigen, should therefore enhance immunity. (Refs 14-16). Furthermore, the optimal vaccine strategy would target antigen delivery, recruit immune cells to the vaccine site, and induce DC maturation. Targeting receptors which regulate chemotaxis has the potential to fulfill all three requirements.

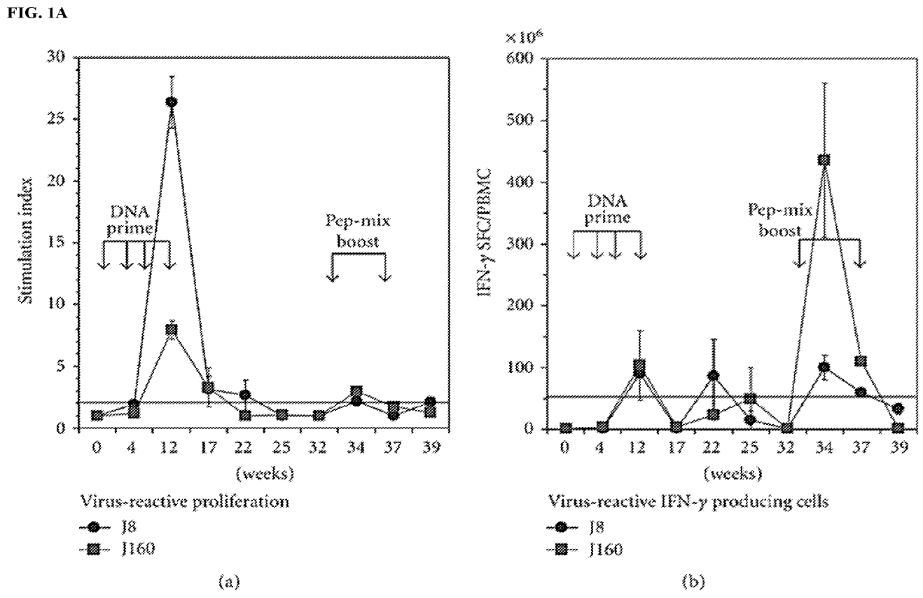

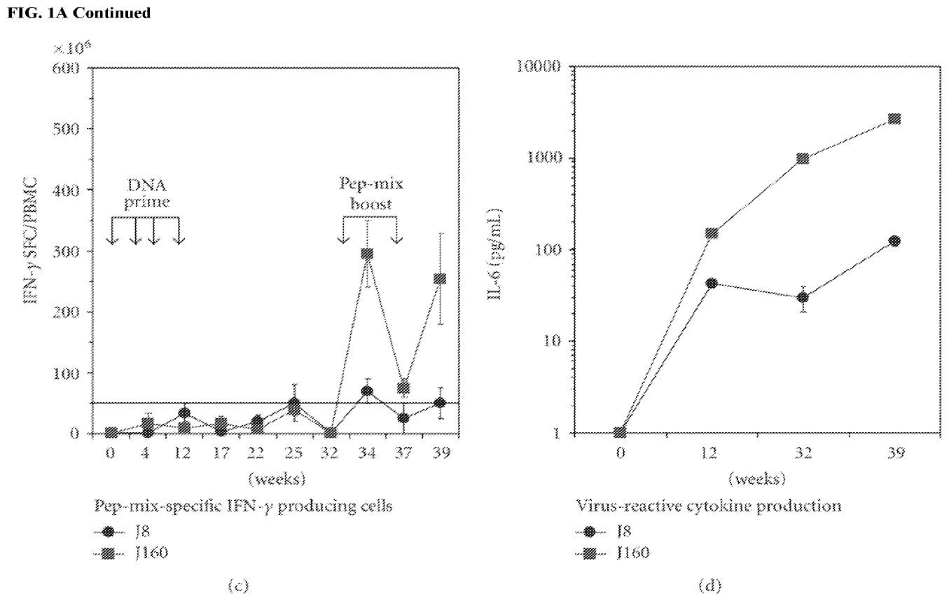

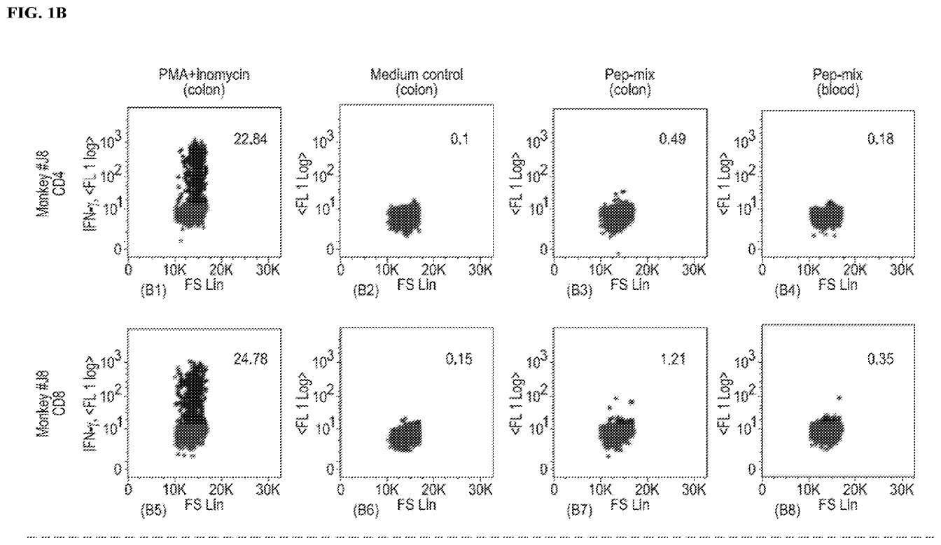

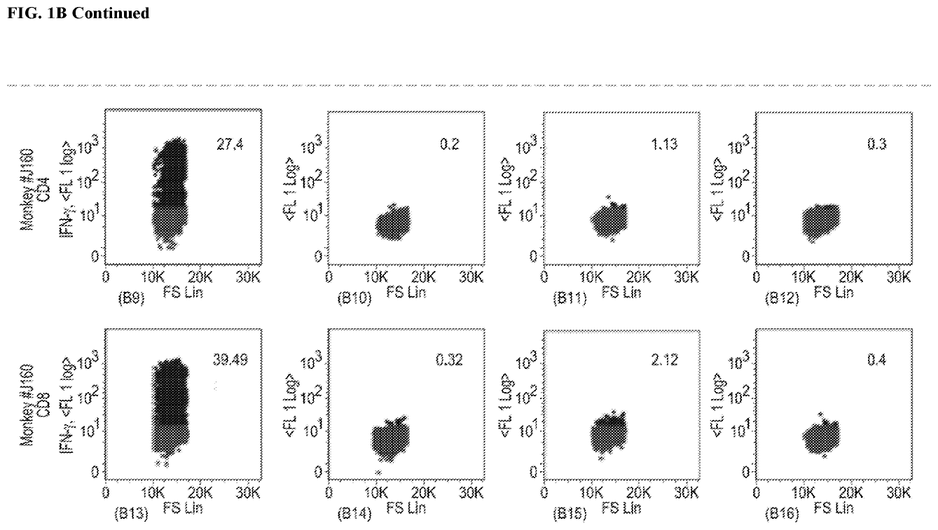

Indeed we previously demonstrated the potential of chemokine fusions as a general strategy for DNA vaccine design by showing induction of HIV env-specific cytotoxic T lymphocytes after DNA vaccination with HIV gp120-chemokine fusions. (Ref 17). We were the first to demonstrate that mice immunized with DNA encoding gp120 fused with proinflammatory chemoattractants of immature dendritic cells, such as β-defensin 2, monocyte chemoattractant protein-3 (MCP-3/CCL7) or macrophage-derived chemokine (MDC/CCL22), elicited anti-gp120 antibodies with high titers of virus-neutralizing activity. The immunogenicity and neutralizing activity was further augmented with the use of chemokine fusion constructs with gp140 (gp120 linked to the extracellular domain of gp41 via a 14-amino acid spacer peptide sequence). Both systemic and mucosal CD8+ cytolytic immune responses were elicited in mice immunized with DNA expressing MCP-3 or β-defensin 2 fusion constructs but not in mice immunized with DNA encoding gp120 alone. (Ref 17). Therefore, the potential for broad application of this approach lies in the induction of mucosal CTL and neutralizing antibodies to HIV-1 envelope, both key requirements for prevention of viral transmission. This MCP3-fused gp120 DNA vaccine also elicited virus-specific T-cell immunity in rhesus macaques. DNA vaccination induced virus-reactive T cells in peripheral blood, detectable by T cell proliferation, IFN-γ ELISPOT, and sustained IL-6 production. Long-term and peptide-specific mucosal memory T-cell immunity was detected in both vaccinated macaques after one year (FIGS. 1A-1B). (Ref 18).

In addition, our prior studies with chemokine-antigen fusion DNA vaccine strategies have demonstrated specific antibody and T cell responses and therapeutic antitumor activity against multiple cancers in preclinical models. (Refs 1, 3, 4, 19, 20). For example, a DNA vaccine comprised of tumor idiotype (sFv), a single polypeptide consisting solely of V_{H} and V_{L} genes linked together in-frame by a short, 15-amino acid linker (Ref 21), fused to a pro-inflammatory chemokine moiety induced therapeutic immunity against a large tumor challenge (20 times the minimum lethal dose) in mouse studies. (Ref 2). Overall, data with three different classes of chemokine receptor ligands (chemokines (Refs 2, 4), defensins (Ref 1), and viral chemokines (Ref 22)) suggest that these fusion DNA vaccines may potentially trigger type 1, Id-specific T-cell immunity, including receptor targeting on APC, APC activation, and APC recruitment.

### Example 2

Phase 1 Clinical Trial of Chemokine-Antigen Fusion DNA Vaccines

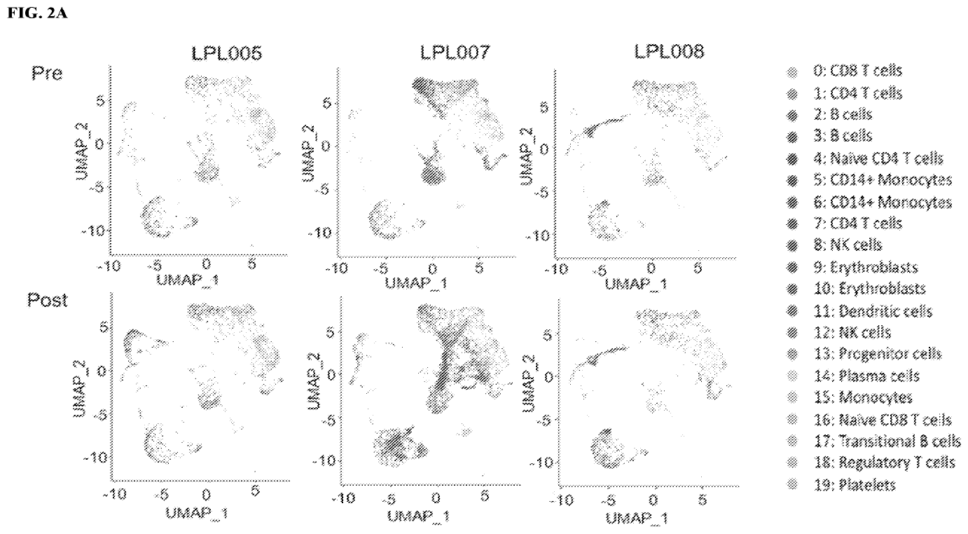

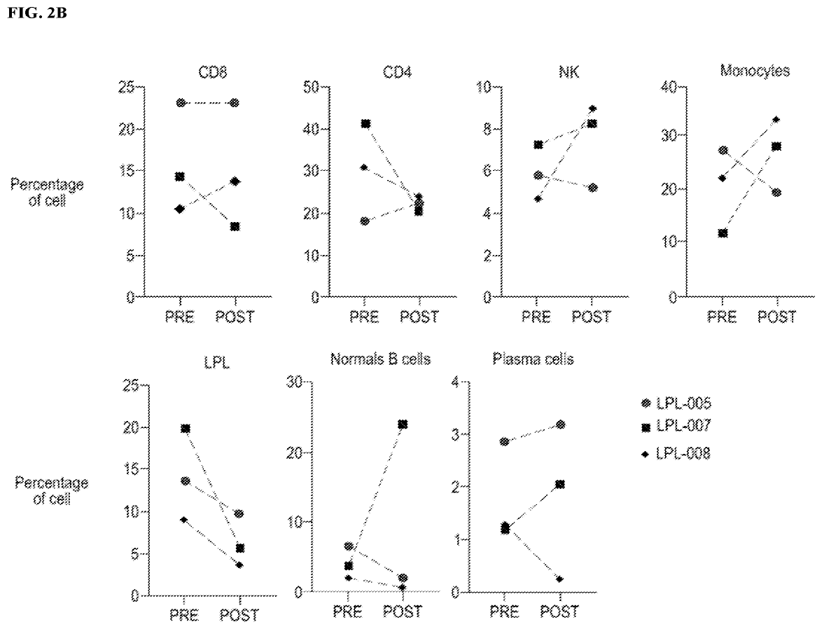

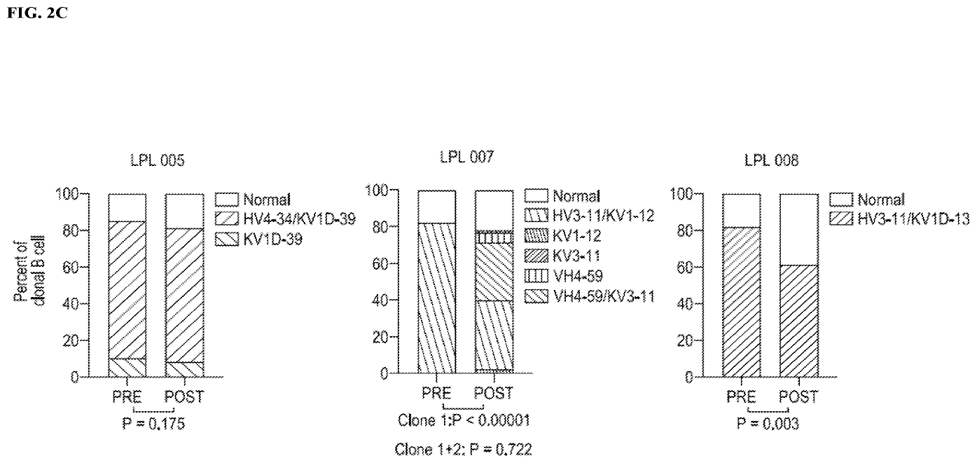

We have recently completed a first-in-human phase I clinical trial of this novel vaccine delivery platform as upfront therapy in patients with asymptomatic lymphoplasmacytic lymphoma (LPL) (ClinicalTrials.gov identifier NCT01209871). We sought to develop a well-tolerated approach to lengthen the indolent phase of LPL without inducing cross-resistance to available cytotoxic therapies. The hypothesis was that anti-tumor immunity could be triggered by targeting APCs in vivo with a chemokine-tumor antigen fusion protein. Patients with LPL received a series of 3 intradermal vaccinations of patient-specific scFv-CCL20 DNA vaccine at 4-week intervals (weeks 0, 4 and 8) using a needle-free device. Two dose levels (500 μg and 2500 μg) were evaluated in a 3+3 dose escalation design. With a median follow up of 14 months (range: 3-30), all pts remain alive. Eight have stable disease (SD); 1 progressed to symptomatic LPL at 8 months after 1^{st} vaccination (LPL 005). All patients completed planned therapy and no dose limiting toxicities were encountered. Idiotype (scFv-CCL20) DNA vaccine therapy appears to be safe in patients with LPL. Importantly, in ongoing correlative studies we have observed by single-cell RNAseq analysis that the tumor immune microenvironment is favorably altered in patients after the chemokine-tumor antigen DNA vaccine treatment. First, despite lack of changes in circulating LPL Ig levels, DNA vaccine treatment significantly reduced the number of clonal LPL tumor cells in the bone marrow compartment in most patients (FIGS. 2A and 2C), representative patients 007 and 008). In addition, comparing pre- vs. post-vaccine time points, vaccine treatment also induced dramatic increases in monocytes in the tumor microenvironment. Clonal diversity of tumor microenvironment T cells is significantly increased after vaccination in LPL patients, except in patients LPL-005 and LPL-009, who both experienced disease progression. (FIG. 2D).

### Example 3

Construction of Chemokine-Fused SARS-CoV-2 Spike (S) Protein DNA Vaccine Candidates

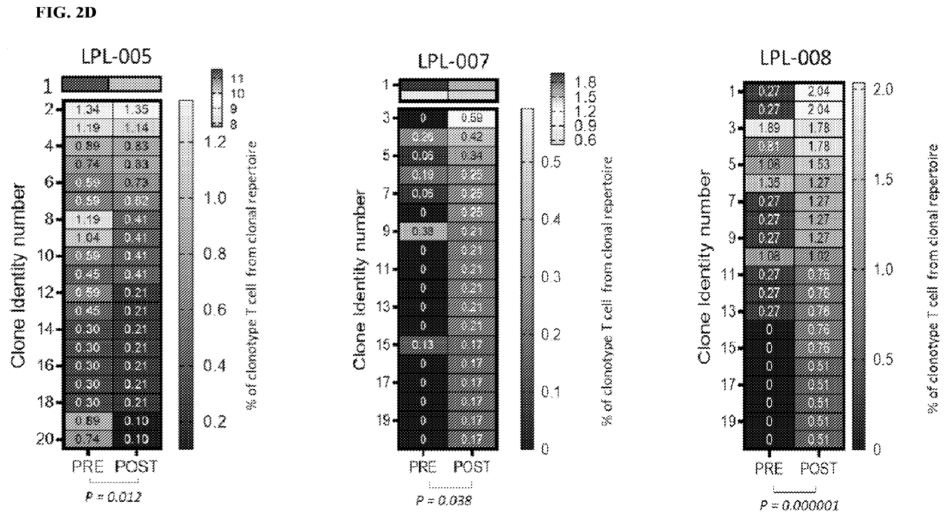

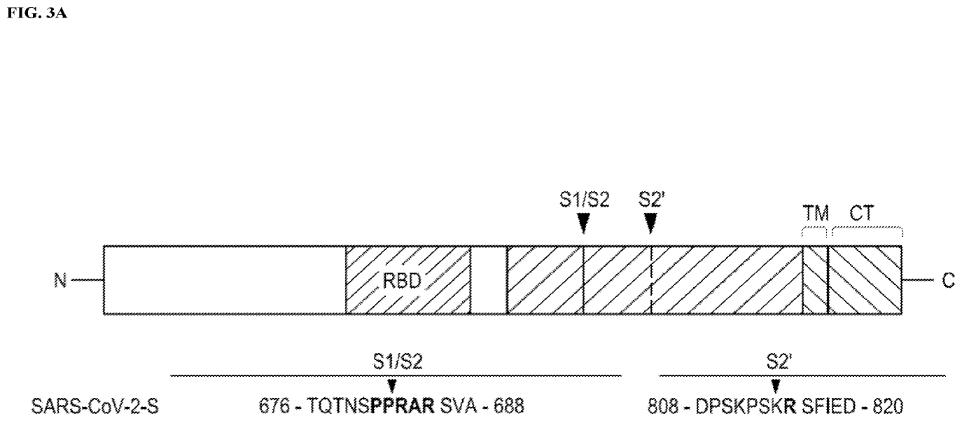

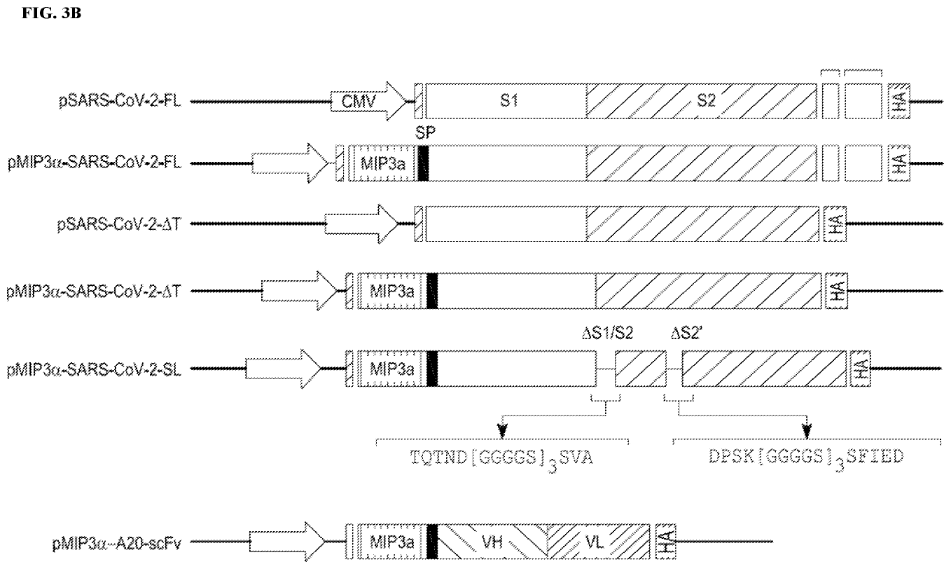

We cloned the SARS-CoV-2 spike (S) gene (FIG. 3A) into our preclinical grade DNA vaccine vector, which contained a mouse MIP3α cassette. We chose the SARS-CoV-2 spike (S) protein (Wuhan-Hu-1 MN908947 strain)(Ref 23) as the target for our chemokine antigen DNA vaccine, primarily because it is known to mediate cellular entry via the ACE2 host cell receptor.

We generated a series of constructs along with appropriate controls (FIG. 3B), to test the generation of neutralizing antibodies and activation of humoral immunity. When generating a SARS-CoV DNA vaccine, Yang et al, Nature, 428(6982):561-564 (2004) found the production of neutralizing antibodies to be dependent upon the inclusion of spike glycoprotein transmembrane (TM) and cytoplasmic (CT) regions. (Ref 24). Notably this region was observed to be similar to the HIV gp41 region we previously showed increased immunogenicity of our HIV DNA vaccine (see Examples 1 and 2). (Refs 17, 25). The pSARS-CoV-2-FL DNA vaccine, which contains the TM and CT regions, was made to serve as a positive control for the production of neutralizing antibodies. The mouse MIP3α cassette necessary for the secretion of chemokine fusion proteins from epidermal cells and delivery to immature dendritic cells (iDCs) was included in a series of constructs including pMIP3α-SARS-CoV-2-ΔT with or without the TM and CT regions postulated to increase neutralizing activity, similar to our results with the pMDCgp140-14 DNA vaccine in a previous HIV DNA vaccine study. (Ref 17). Furthermore, we hypothesized that removing the protease cleavage sites and inserting a flexible linker (2×G4S) in the SARS-CoV-2 gene would modify translational exposure of conformational epitopes of the trimeric spike protein and produce a high titer of neutralizing antibodies. This fusion construct designated pMIP3α-SARS-CoV-2-SL was also tested. We also have the flexibility to generate additional constructs (not shown) based on newly reported mutations in this rapidly evolving field. (Refs 26, 27). The native conformation of the expressed SARS-CoV-2-spike(S) protein trimers and monomers can be verified using the NativePAGE system (ThermoFisherScientific™, Waltham, MA) following the manufacturer's guidelines. The irrelevant lymphoma scFv DNA vaccine pMIP3α-A20-scFv²⁸ can serve as a negative control.

Description of the pSARS-CoV-2-FL construct (SEQ ID NO:1): the human MIP3α secretion leader sequence (SL), without human MIP3α gene, was cloned in-frame with SARS-CoV-2-S gene with the transmembrane (TM) and all the amino acids deleted from the cytoplasmic domain (CT), i.e., the deletion of SEQ ID NO:42. The protein encoded by this construct is set forth as SEQ ID NO:2.

Description of the pMIP3α-SARS-CoV-2-FL construct (SEQ ID NO:3): the human MIP3α, comprising the secretion leader sequence (SL), was cloned in-frame with SARS-CoV-2-S gene with the transmembrane (TM) and all the amino acids deleted from the cytoplasmic domain (CT), i.e., the deletion of SEQ ID NO:42. The protein encoded by this construct is set forth as SEQ ID NO:4.

Description of the pSARS-CoV-2-ΔT construct (SEQ ID NO:5): the human MIP3α secretion leader sequence (SL), without human MIP3α gene, was cloned in-frame with SARS-CoV-2-S gene without the transmembrane (TM) and cytoplasmic domain (CT). The protein encoded by this construct is set forth as SEQ ID NO:6.

Description of the pMIP3α-SARS-CoV-2-ΔT construct (SEQ ID NO:7): the human MIP3α, comprising the secretion leader sequence (SL), was cloned in-frame with SARS-CoV-2-S gene without the transmembrane (TM) and cytoplasmic domain (CT). The protein encoded by this construct is set forth as SEQ ID NO:8.

Description of the pSARS-CoV-2-ΔTS construct (SEQ ID NO:9): the human MIP3α secretion leader sequence (SL), was cloned in-frame with genetically modified SARS-CoV-2-S gene, in which the two protease cleavage sites were removed (ΔS1/S2: removed RRAR amino acids and ΔS2′: removed PSKR (SEQ ID NO:21) amino acids). The protein encoded by this construct is set forth as SEQ ID NO:10.

Description of the pMIP3α-SARS-CoV-2-ΔTS construct (SEQ ID NO:11): the human MIP3α, comprising the secretion leader sequence (SL), was cloned in-frame with genetically modified SARS-CoV-2-S gene, in which the two protease cleavage sites were removed (ΔS1/S2: removed RRAR (SEQ ID NO:20) amino acids and ΔS2′: removed PSKR amino acids). The protein encoded by this construct is set forth as SEQ ID NO:12.

Description of the pSARS-CoV-2-ΔTSL construct (SEQ ID NO:13): the human MIP3α secretion leader sequence (SL), was cloned in-frame with a genetically modified SARS-CoV-2-S gene, in which the two protease cleavage sites (ΔS1/S2 and ΔS2′) were removed and replaced with flexible linker (G₄S)₃ inserts. The protein encoded by this construct is set forth as SEQ ID NO:14.

Description of the pMIP3α-SARS-CoV-2-ΔTSL construct (SEQ ID NO:15): the human MIP3α, comprising the secretion leader sequence (SL), was cloned in-frame with a genetically modified SARS-CoV-2-S gene, in which the two protease cleavage sites (ΔS1/S2 and ΔS2′) were removed and replaced with flexible linker (G₄S)₃ inserts. The protein encoded by this construct is set forth as SEQ ID NO:16.

All the constructs described above were cloned in the pUMVC3 vector, described in Example 10.

### Example 4

Evaluation of vaccine candidates for their immunogenicity and antigenicity by characterization of SARS-CoV-2-spike-specific antibody and T-cell responses

We immunized 6- to 9-week-old female BALB/c mice with the DNA vaccines (SA1a) by injections of 100 μg of DNA resuspended in 50 μL of PBS in each quadriceps muscle 5 times every 2 weeks. Two weeks after the final immunization, the presence of anti-SARS-CoV-2 S protein antibodies in immunized sera was assayed by ELISA in a 96-well plate coated with human recombinant SARS-CoV-2 S protein (The Native Antigen Company Ltd, Oxfordshire, UK). The bound antibodies were detected by goat anti-mouse IgG(FC)-HRP via ELISA (ThermoFisher Scientific™, Waltham, MA). In separate experiments, T-cell mediated immunity was assessed by examining the activation of SARS-CoV-2 S protein specific T-cells, and exemplary results are shown in Example 9.

### Example 5

Determination of the Ability of pMIP3α-SARS-CoV-2 DNA-Immunized Mice to Functionally Inhibit SARS-CoV-2 infection In Vitro and In Vivo

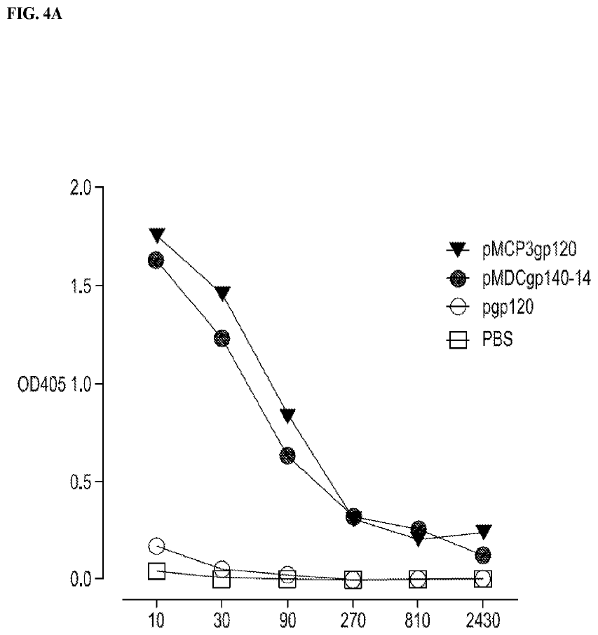

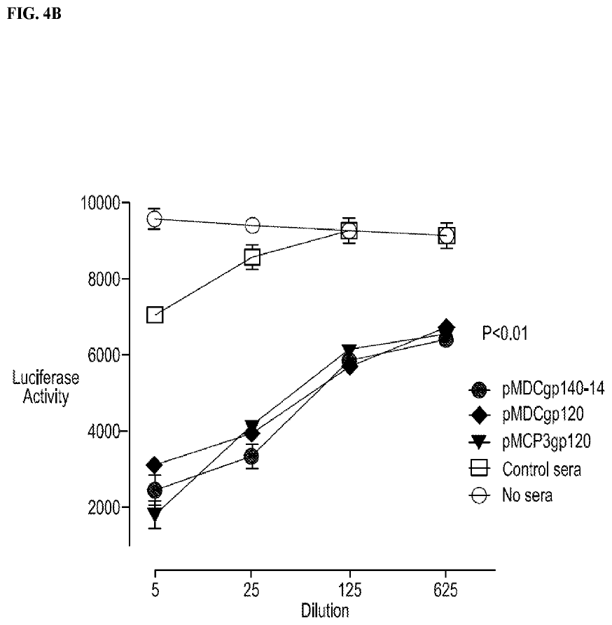

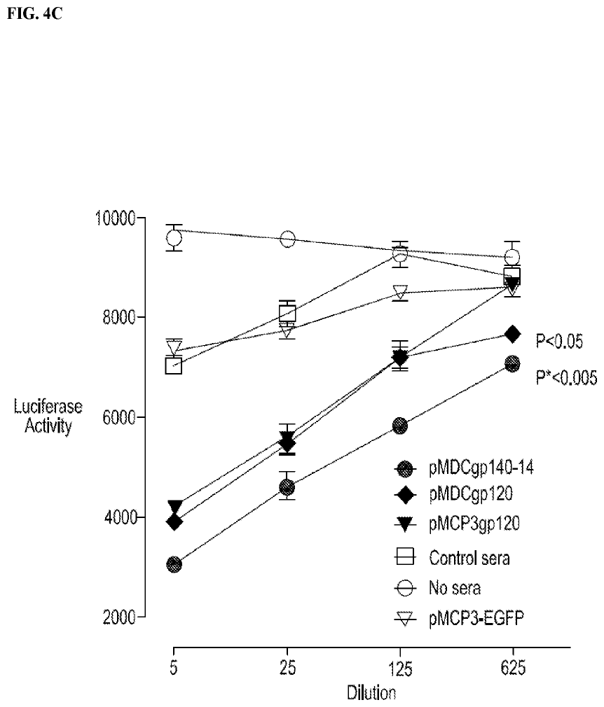

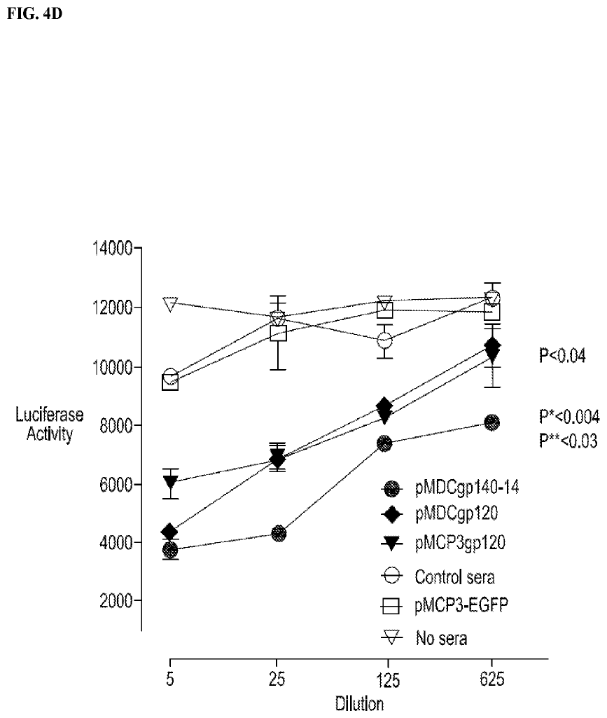

We will develop pseudoviruses for the antibody neutralization assays in vitro and in vivo, for SARS-CoV-1, and various mutations of SARS-CoV-2 detailed below. The SARS-CoV-2 virus genome is prone to mutations that lead to escape from immune recognition. (Ref 31). The D614G spike protein mutation is present in 29% of global samples. There are three mutational sites, V367F, G476S, and V483A within the RBD domain (Los Alamos SARS-CoV-2 mutation analysis pipeline: cov.lanl.gov/). (Ref 27). Of these, only G476S occurs directly at the binding interface of the RBD and the ACE2 peptidase domain. Therefore, as many virus variants are found now and will appear in the future, it is necessary to develop an “off-the-shelf” universal vaccine, able to inhibit infection from a broad range of SARS-CoV-2 mutations. Previously, we demonstrated sera collected from mice immunized with a chemokine-HIV gp140-14 DNA vaccine inhibited HIV-Env-mediated cell fusion and infection of pseudovirus expressing not only the same Env from the 89.6 isolate, but also HIV-1 pseudotype virus expression of various Env 89.6 ([for R⁵×4]: FIG. 4B), NL4-3 ([for R⁵]: FIG. 4C) JRFL ([for R⁵]: FIG. 4D) despite different coreceptor usage. (Ref 17).

Our central hypothesis is that the chemokine fusion SARS-CoV-2 DNA vaccine can elicit distinct and broadly-neutralizing antibody responses against various mutations of SARS-CoV-2. We will test whether sera from pMIP3α-SARS-CoV-2SL DNA-immunized mice can inhibit infection by the SARS-CoV-1, and various SARS-CoV-2 pseudoviruses in vitro. Lentivirus-based VSV-G, SARS-CoV-1 and SARS-CoV-2 (Wuhan-Hu-1 MN908947, D614G, V367F, G467S, and V483A) pseudotype viruses will be prepared as previously described. (Ref 17). Viral stocks will be prepared by transfecting 293T cells with plasmid encoding the various viral spike proteins and luciferase reporter. This assay is sensitive/quantitative and can be conducted in biosafety level-2 facilities. The virus will be preincubated with titrated amounts of sera from immunized mice for 1 hour at 37° C. Cells will then be infected with the pseudovirus for 4 hours and washed prior to lysis at 48 hours. The lysate will be assayed for luciferase activity.

### Example 6

Establishment of SARS-CoV-2 Pseudovirus Infection Murine Models for In Vivo Evaluation of the SARS-CoV-2-Spike DNA Vaccine

Because experiments with the live SARS-CoV-2 virus require Biosafety Level-4 (BSL-4) facilities, which has restricted the development of anti-SARS-CoV-2 vaccines, we will use a lentivirus-based pseudovirus cell infection assay. This assay is widely used for viral entry studies in BSL-2 conditions. In this study, we will develop in vivo pseudo-SARS-CoV-2 virus infection models.

First, we will develop and optimize a bioluminescent imaging murine model for the SARS-COV-2 pseudovirus, to assess whether the bioluminescent signal of the SARS-CoV-2 pseudovirus (containing the luciferase reporter gene) can be detected in mice. K18-human ACE2 transgenic mice (The Jackson Laboratory, Bar Harbor, Maine) will be administered the pseudovirus in different dosages via different routes including intranasal injection and will be followed by bioluminescent imaging at several time points. We are also looking for the optimal SARS-CoV-2 strain pseudovirus candidate, among the SARS-CoV-2 mutations, to utilize in the following in vivo challenge study. We will choose the optimal time point at which we can observe peak luciferase intensity. Finally, we will statistically determine whether the inoculated viral doses correlate with the bioluminescence values of the infected mice. Second, to confirm in vitro observations, we will evaluate inhibitory activities of immunization by our DNA vaccine in in vivo pseudovirus infection mouse models. For this experimental objective, control groups of K-18 transgenic mice will receive five immunizations at five separate time points of the DNA plasmids used in our in vitro study (FIG. 3B) every other week. Two weeks after the last vaccination, all mice will be challenged with the previously selected optimal SARS-CoV-2 pseudovirus. We will track the inhibitory effects of our pMIP3α-SARS-CoV-2-S DNA vaccine through bioluminescent imaging at different time points after pseudovirus injection with the AmiX imaging system as described previously. (Ref 32).

### Example 7

Performing IND-Enabling Studies with Lead Clinical-Grade Vaccine Candidate

The lead candidate for a chemokine-fused SARS-CoV-2 S protein DNA vaccine identified in SA1 will be adapted for clinical work up by cloning the SARS-CoV-2 S protein sequences into the same clinical grade vector (FIG. 5), which was previously used in our phase I trial in patients with LPL. (Ref 5).

To prepare for IND filing we will follow the practices set out in the U.S. FDA guidance on plasmid DNA vaccines for infectious disease indications. These aim to establish quality and consistency of plasmid manufacture, and ensure the completion of extensive preclinical safety studies. Standard operating procedures (SOPs), which already exist in support of the clinical trial in LPL patients, will be adapted for the process development, pilot scale and scale-up expression production, assay development, technology transfer, cGMP manufacturing, formulation and stability profiling of the lead COVID-19 vaccine candidate in anticipation of clinical trial design and initiation. The source and genotype of the E. coli used to establish the Master Cell Bank (MCB) and Working Cell Bank (WCB), as well as the procedures to construct master and working cell banks used for production will be documented. These cell banks will be tested for bacteriophage and other adventitious agent contamination.

Proper insert orientation and identity of the MIP3α-SARS-CoV-2 S fusion gene will be verified in individual clones by restriction mapping and DNA sequencing. Potency will be assessed by Western blotting, to validate the expression of the correct size fusion protein in 293T cells. After the product is tested for identity and potency, the plasmid will be released to GMP manufacturing. SOPs for process development and small scale GMP manufacturing will be performed. Large-scale production of clinical-grade plasmid DNA, along with purification and quality assurance analyses, will be performed according to SOPs, and 3 consistency lots will be prepared to demonstrate acceptable compliance and reproducibility. The final product will be tested for plasmid identity by sequencing, sterility, endotoxins (not to exceed 40 EU/mg plasmid by the Limulus Amebocyte Lysate [LAL] test), levels of residual E. coli RNA (<1%) and genomic DNA (<1%), and percentage of supercoiled plasmid purity (>80%), all in compliance with FDA guidance for clinical trials. Stability testing of the GMP material at −20° C. and 2°−8° C. will be carried out at 3, 6, 9, and 12 months.

Subsequently, preclinical toxicity and biodistribution/persistence studies will be carried out to evaluate the formulation and method of administration proposed for the clinical study. Studies will assess whether modulation of cellular or humoral components of the immune system might result in unintended adverse consequences, such as generalized immunosuppression, chronic inflammation, autoimmunity or other immunopathology. Based on our prior experience, using the same plasmid backbone (pUCMVC3), the vaccine is expected to result in minimal toxicities. (Ref 5). The most likely anticipated reactions include local erythema and induration at the sites of injection and transient flu-like symptoms. No known oncogenic or immunomodulatory sequences are detected in the plasmid. Because we are using a plasmid vector previously documented to have an acceptable biodistribution/integration profile, biodistribution studies are unnecessary.

### Example 8

Statistical Methods

In vivo studies will be performed as randomized experiments done concurrently with controls and repeated to provide sufficient statistical power for analysis. Two sample t-test will be used to compare serum protein antibody/spleen T cell response (SA1b) and serum neutralizing antibody (SA1c) from wild type mice treated with the 2 active vaccine plasmids (pMIP3α-SARS-CoV-2-S and pMIP3α-SARS-CoV-2-SL) and the 4 control plasmids. With 10 mice/group, the power will be of 84% to detect a difference of 1.4 SD (standard deviation) in mean between vaccine and control at 1-sided alpha of 0.025 (which accounts for 2 active groups). To compare between the two active vaccines where the difference might be smaller, data from the 3 repeats may need be combined. A sample size of 30 per group will have 81% power to detect a difference of 0.75 SD between the two active vaccines at 2-sided alpha of 0.05. Linear mixed effect models will be used to examine and compare the daily luciferase activity after pseudovirus challenge in K18-human ACE2 transgenic mice that are vaccinated (SA1c). Treatment, time, and their interaction will be modeled as fixed effects and individual mouse as random effect. The effect of time as a linear function or nonlinear function will be explored. With 5 mice per group, the power will be 82% to detect a difference of 2.1 SD between vaccine and control at 1-sided alpha of 0.025. Combining 3 repeats with 15 mice/group, the power will be 82% for detecting a difference of 1.1 SD between the 2 active vaccines.

### Example 9

Development of Chemokine-Antigen SARS-CoV-2-(S) DNA Vaccine and Testing Immunogenicity in Wild-Type Mice

All wild type mice have been successfully immunized with all four viable SARS-CoV-2 DNA vaccine candidates listed: pSARS-CoV-2-FL, pMIP3α-SARS-CoV-2-FL, pSARS-CoV-2-ΔT, pMIP3α-SARS-CoV-2-ΔT.

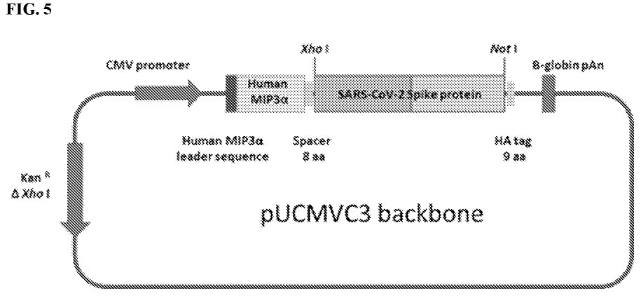

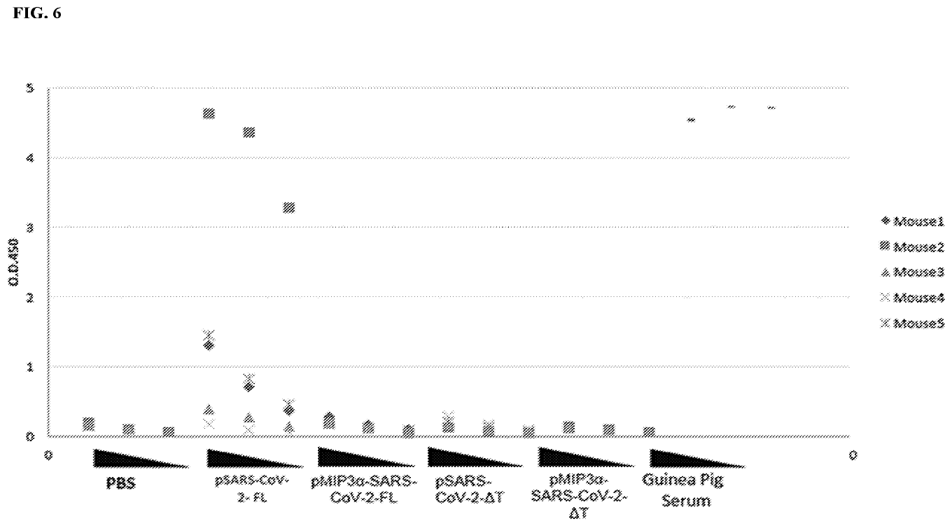

Sera collected two weeks after the fifth and final vaccination (after all mice were euthanized) was tested via ELISA for humoral responses against the SARS-CoV-2 spike antigen. As seen in FIG. 6, only the pSARS-CoV-2-FL vaccine group elicited a strong neutralizing antibody response against the SARS-CoV-2 antigen. A PBS injection group was included as a negative control, with a Polyclonal Anti-SARS Coronavirus Guinea Pig antiserum (BEIResources, Catalog #NR-10361) acting as a positive control.

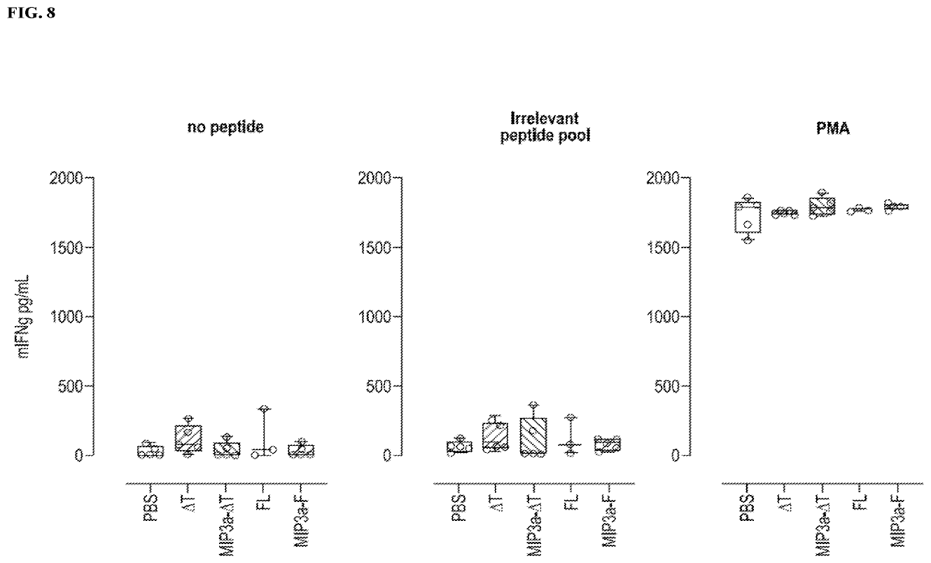

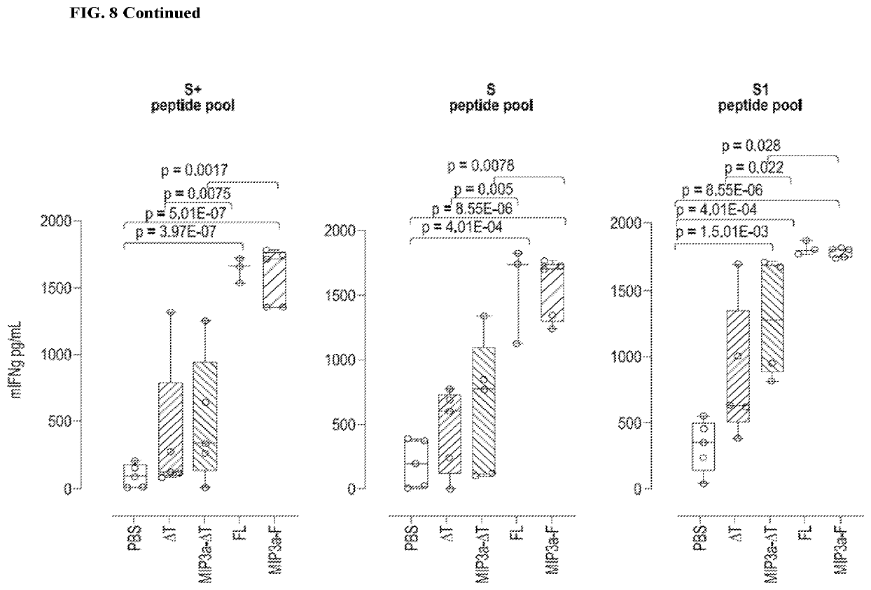

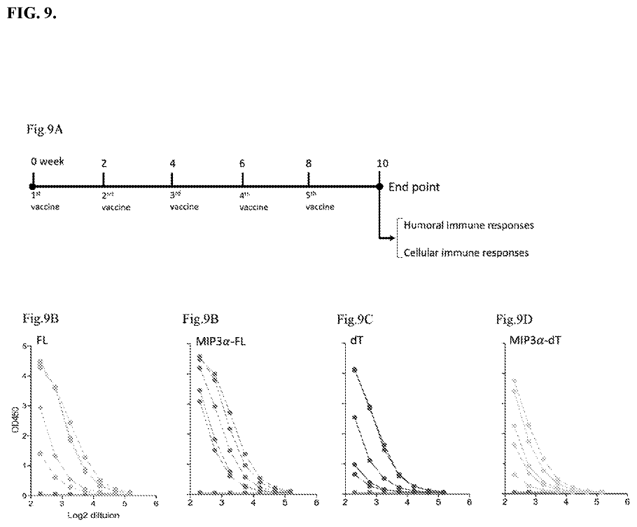

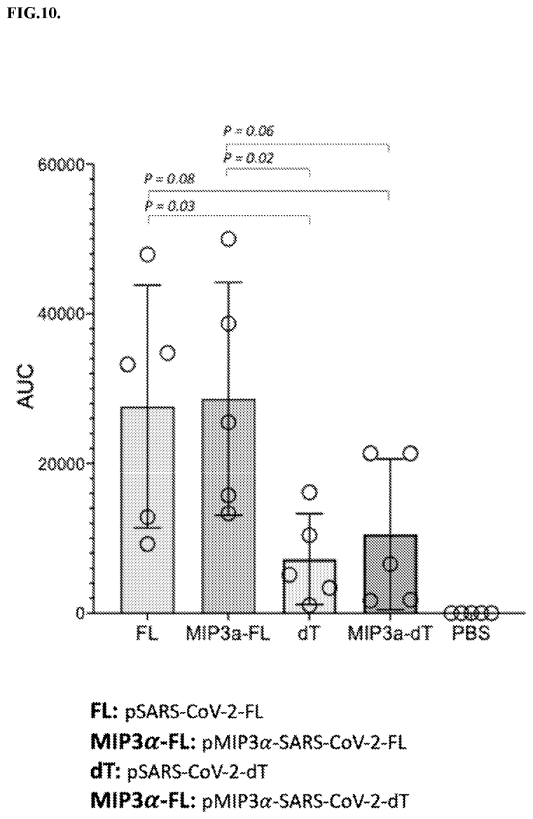

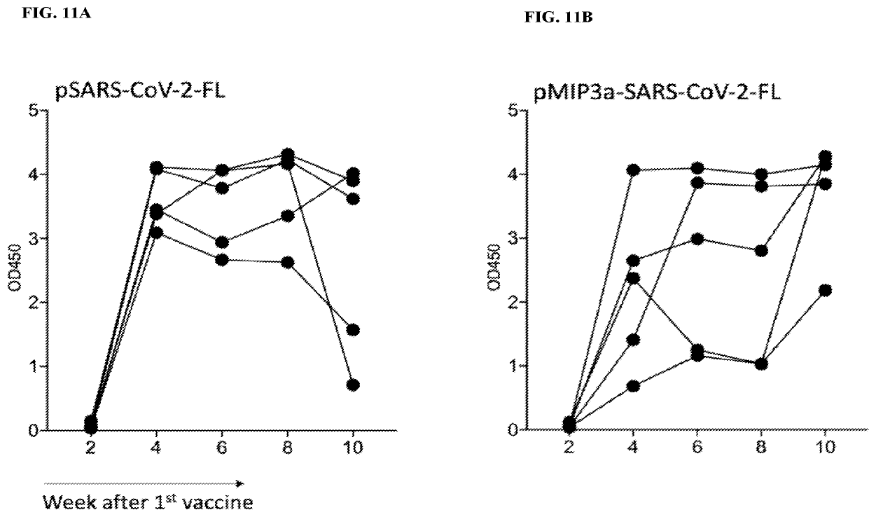

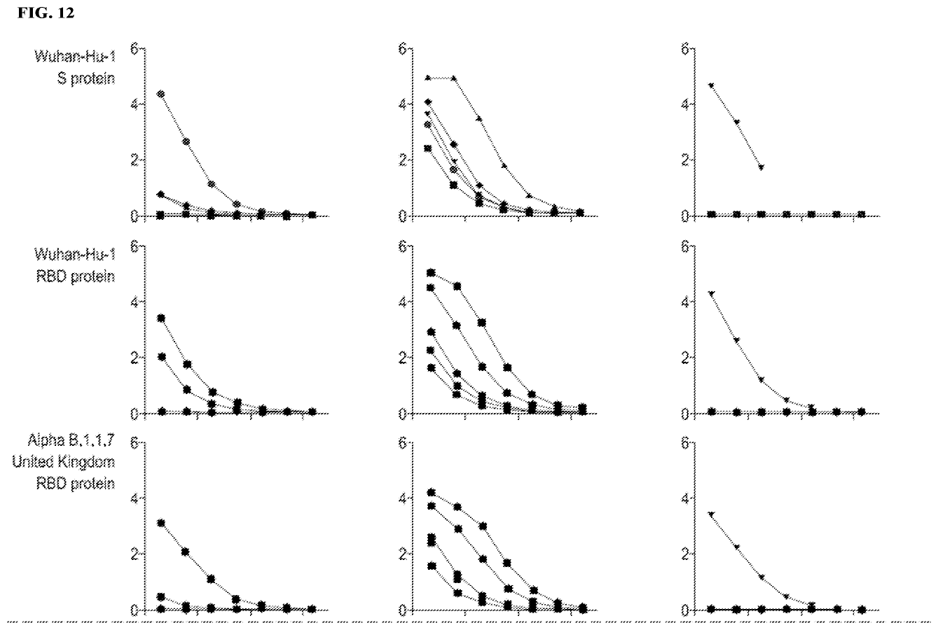

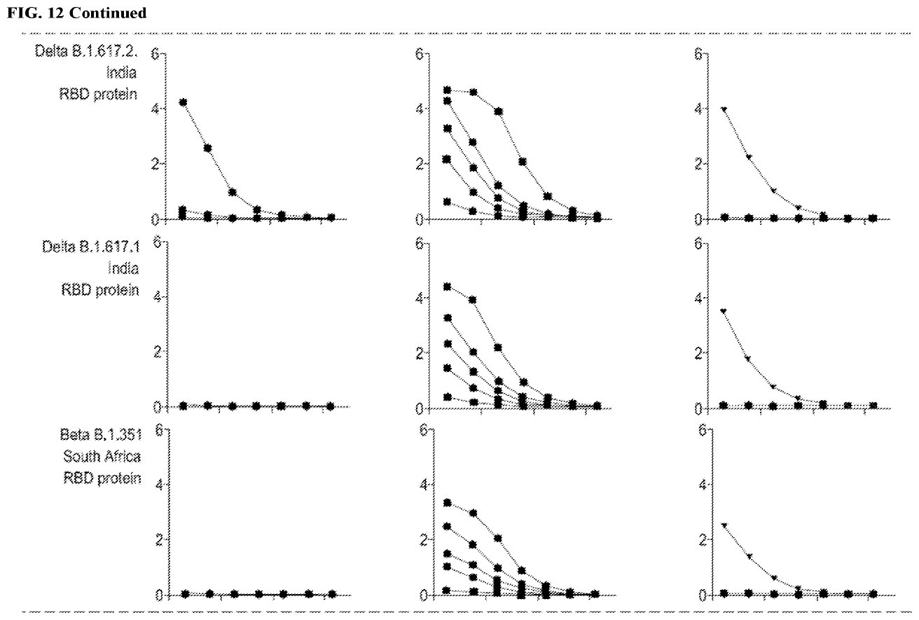

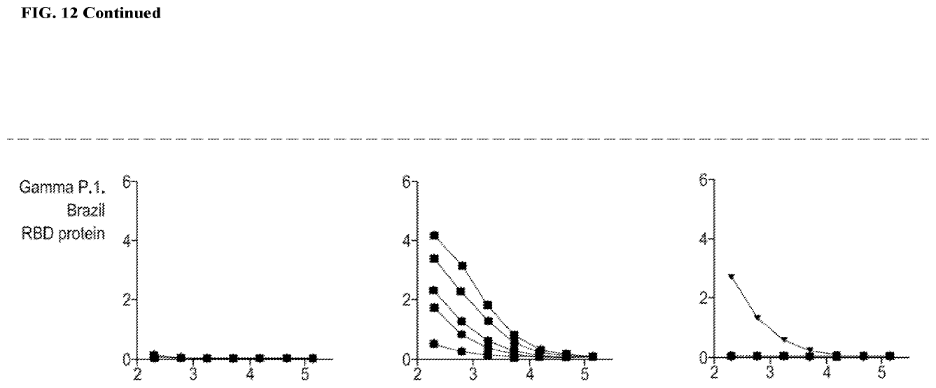

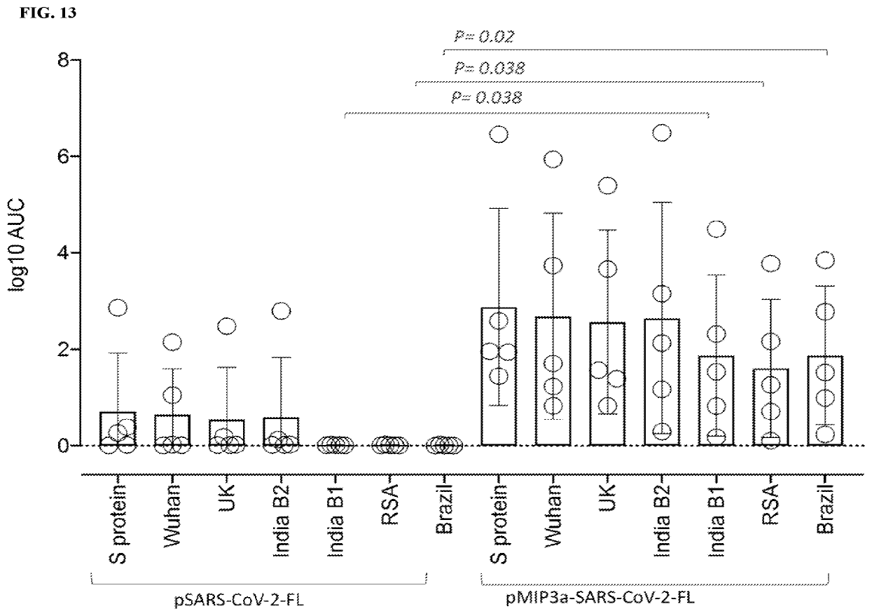

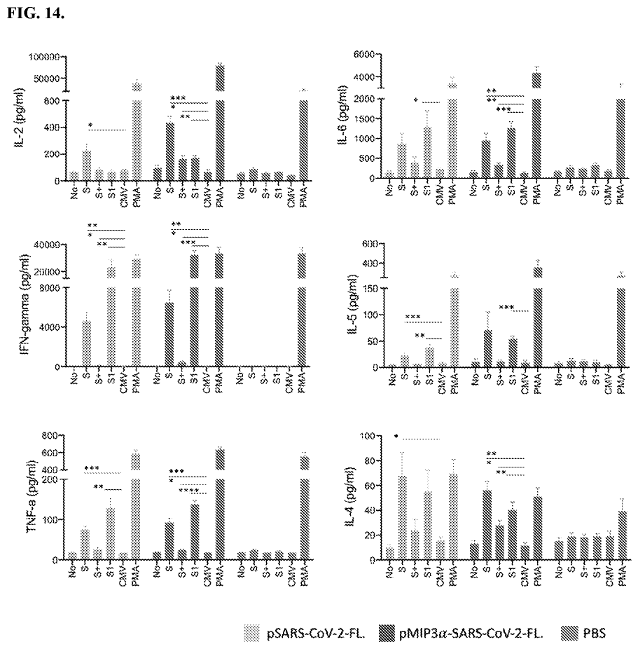

To determine the immunogenicity of our 4 DNA Vaccine constructs, SARS-CoV-2 spike protein-specific T cells were evaluated after the fifth immunization by ex vivo antigen stimulation. Spleenocytes (1×10⁶) from immunized mice were incubated in v-bottom wells in the presence of 2 μg/ml of S1, S+, or S peptide pools (S1: spike protein 1-692aa peptide pools; S+: spike protein 689-895aa peptide pools; S: 304-338aa, 421-475aa, 492-519aa, 683-707aa, 741-770aa, 785-802aa and 885-1273aa peptide pools), stimulation with irrelevant antigen, or without stimulation. 48 hours later, the cell supernatant was collected. The levels of INF-γ secreting Spike antigen T cells were measured in all vaccine groups immunized with S-encoding DNA vaccine using Quantikine ELISA kit (R&D systems) according to the manufacturer's recommendations. The results (FIG. 8) showed that vaccine groups pSARS-CoV2-FL and pMIP3α-SARS-CoV-2-FL induced a statistically significant T cell response in Balb/c mice when compared with the PBS, pSARS-CoV-2-ΔT, and pMIP3α-SARS-CoV-2-ΔT groups. No significant difference was observed between the pSARS-CoV2-FL and pMIP3α-SARS-CoV-2-FL vaccine groups, suggesting that induced immunogenicity from our SARS-CoV-2 DNA vaccine is dependent on the inclusion of the transmembrane domain.

In a follow-up study, we will optimize the delivery system for the SARS-CoV-2 DNA vaccine plasmids by utilizing a TROPIS needleless injector (currently under MTA negotiations with Pharmajet). The standardized delivery system will ensure Intramuscular (IM) delivery of our desired vaccine plasmids to alternating left and right thigh muscles in wild type mice. Secondly, in previous lymphoma preclinical studies published by our group, we have evidence that administration of low doses of cardiotoxin at vaccination sites potentiate antigen specific T-cell immunity induced by genetic cancer vaccines in mice. (Ref 3). Our previous studies have suggested that cardiotoxin-induced sterile inflammation generates a favorable microenvironment that promotes multiple stages in the development of adaptive immunity. (Refs 3, 33). We will inject mice with a combined cardiotoxin and DNA Vaccine therapy, and evaluate the immunogenicity induced via this modified protocol.

### Example 10

pUMVC3 cloning. The pUMVC3 plasmid was a derivative of a pUC19-based plasmid and contains the cytomegalovirus immediate early promoter-enhancer with a partially deleted intron A and rabbit β-globin polyadenylation signal flanking a polylinker for insertion of heterologous open reading frames, as well as the kanamycine resitance gene. This vector was generated by the University of Michigan for human gene therapy studies. DNA vaccines constructed with this vector have been approved by FDA for human studies. (Ref 34).

The pUMVC3 plasmid comprised the CMV promoter-enhancer and CMV IE 5′ UTR. The IE1 human cytomegalovirus immediate-early (hCMV IE1) enhancer/promoter was known to be one of the strongest enhancer/promoters known and was shown to be active in a broad range of cell types. The hCMV enhancer/promoter promoter region included a tissue-specific modulator, multiple potential binding sites for several different transcription factors, and a complex enhancer (U.S. Pat. No. 5,688,688).

The pUMVC3 plasmid further comprised the CMV IE Intron A. The Intron A region of the hCMV IE1 enhancer/promoter has been shown to contain elements that enhance expression of heterologous proteins in mammalian cell. (Refs 35, 36). The hCMV IE1 was deleted in part by removal of a 555-bp XcmI-HpaI fragment to increase expression from the promoter (U.S. Pat. No. 6,893,840).

The pUMVC3 plasmid further comprised the β-globin polyadenylation sequence (pAn). The rabbit β-globin 3′ untranslated region (3′-UTR) and polyadenylation sequence was shown to allow efficient arrest of the transgene transcription. (Ref 37).

The pUMVC3 plasmid further comprised an origin of replication (Ori). In this plasmid, a minimal E. coli origin of replication was chosen to limit vector size, while having the same activity as the longer Ori from the original pUC19 plasmid. Origin of replication coordinates included the region from the −35 promoter sequence of the RNA II transcript to the RNA/DNA switch point. (Ref 38).

The pUMVC3 plasmid further comprised a Kanamycin resistance gene (KanR). Kanamycin is inactivated by bacterial aminophosphotransferases (APHs). Resistance to Kanamycin was conferred by KanR gene from Streptomyces kanamyceticus ISP5500. (Ref 39).

With regards to the different SARS-CoV spike protein DNA sequences cloned or to be cloned into pUMVC3, the sequences were or will be codon-optimized for an effective protein expression of the spike protein after transfection of the different construct in the human cells.

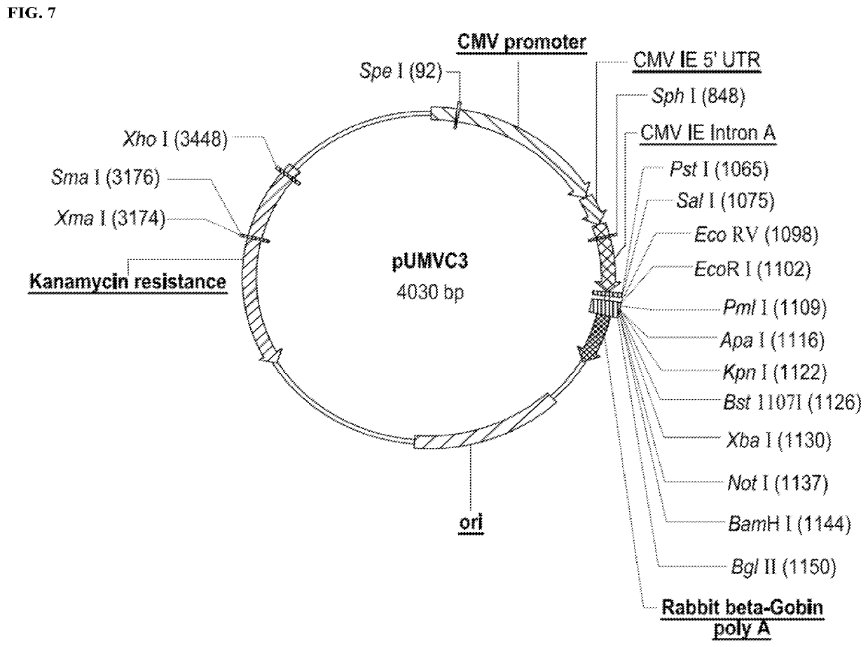

The map of the pUMVC3 plasmid is shown on FIG. 7.

Informal Sequence Listing

```
SEQ ID NO: 1 = pSARS-COV-2-FL plasmid DNA sequence. The underlined portion
shows the DNA sequence coding for a modified SARS-COV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcgtgaatttgaccaccaggacccagcttccgcctgcctataccaattcatttaccagagg
tgtatattacccggataaagtgttccgatcctcagttctccattctacacaagacttgttcctccccttcttctcaaatgtgacttggttt
cacgcaatccatgtgtctggaacgaatggcaccaagaggtttgacaatccagtcttgccttttaatgatggtgtctatttcgctagca
cggaaaaaagcaatataatacggggttggatcttcggcactacacttgacagtaaaacgcagagccttcttatagtgaataacgct
accaacgtggtaataaaagtgtgtgagttccaattctgcaatgatccattcttgggcgtgtattaccataaaaataacaaaagttgg
atggaaagcgaattccgcgtgtacagctctgccaacaactgcacttttgagtatgtgagccagccatttttgatggatctggaagga
aagcaaggtaactttaaaaacttgcgggagttcgtttttaagaacatcgatgggtatttcaaaatctactcaaaacacactcctatta
atttggttcgcgatttgccacagggtttctctgccctcgaacctcttgttgatctccctattggaataaatatcacgagattccagacat
tgctcgccctgcatcgctcatatctgacacctggtgatagcagcagcggatggacggcaggcgcagccgcgtattacgttggctac
ttgcaacccagaacatttctccttaagtataatgagaatggtacgatcactgatgcggtcgactgcgcgttggaccctctgagcgag
acaaaatgcactctcaagagtttcactgtcgagaaaggcatttaccaaacatctaatttccgagtgcaaccaactgagtccattgtt
cgattcccaaacattactaacttgtgtcctttcggcgaagtgttcaacgcaacgagatttgcaagtgtatacgcgtggaaccgaaag
agaatcagcaactgttagccgattattctgttctgtacaatagcgctagcttcagcactttcaaatgctacggtgtaagtcctacaa
agctgaatgacctctgtttcacaaatgtgtacgcggatagcttcgtcattaggggagatgaagtacggcaaatcgcccccggtcaa
acagggaagatagctgactacaactataaacttccggatgattttacggggtgtgttatagcgtggaattctaacaacctcgattca
aaggtcgggggaaattacaattacctttaccgcctgttccgaaagtcaaatcttaagccattcgaaagggacatatcaacggaaat
ttatcaagccgggtctactccatgcaatggcgtcgagggttttaattgctattttccactgcaatcttacggtttccaaccaacgaacg
gagtaggatatcaaccttaccgggttgtagtgttgtctttcgaactgctccacgcgccagctaccgtgtgcggtccgaaaaaaagca
caaatctggttaagaacaagtgtgttaacttcaactttaacggtctcactggaacaggtgtacttacagaatccaataagaaatttct
tccgttccaacagttcggtagagacattgcggacaccactgatgctgtgcgagatcctcagacactcgaaatactggacattacac
cttgcagcttcggcggtgttagtgtaatcacgccgggcactaatacatccaatcaggtcgctgtgttgtaccaagatgttaactgtac
ggaggtacctgttgcaatacatgcggaccaactcactcctacttggagggtatattctaccggatccaacgtgttccagactcgggc
gggctgcctgataggtgccgagcatgtcaataattcttacgaatgcgatattccaataggggccggcatatgtgcatcttatcaaac
ccaaaccaactccccccgacgcgcccgctctgttgcgtcacagagtattatagcctatacgatgtctttgggggcggaaaactccgt
cgcttactccaacaacagtatagcaatccctacgaattttaccatctccgtaacaactgaaatccttcctgtgtcaatgaccaaaact
tctgtcgattgcacgatgtatatctgcggagacagcacggaatgtagtaacttgctcctccagtacggctctttctgcacacagctca
atcgggcgctgaccgggatagcggtggaacaagacaagaacacccaggaagtcttcgcgcaggtcaagcaaatctacaagacc
cctcccattaaagattttggtggttttaactttagccaaatactgccagatccgtccaaaccctccaaacgatccttcatcgaggacct
cctttttaacaaagttaccctcgccgacgccgggttcataaaacagtacggcgactgcttgggtgacatcgcagccagagacttgat
ttgtgcgcaaaaattcaacggccttacggtcctgcctccattgctgaccgatgaaatgattgcccagtatacctctgctttgttggcag
ggacaataacttctgggtggacgtttggagcaggcgctgccctgcagatcccttttgccatgcagatggcataccggtttaacggga
tcggtgttactcagaacgtactctatgagaatcaaaagctgatcgccaaccaatttaatagcgccatcggtaaaatccaggatagtt
tgagcagcaccgcttctgcacttgggaagcttcaggatgttgtgaatcagaacgcccaagcattgaatactctcgtcaaacaattga
gtagtaatttcggagccatctcatctgtgttgaatgacatactcagtcgcctcgataaggtagaggctgaggtgcagatagaccggc
ttataactggtagattgcagagccttcagacttacgtgacgcaacagcttatccgagcagcagaaatcagggcatcagcaaatctt
gccgccaccaaaatgtcagagtgcgtactgggccaaagtaaaagagtggatttttgtggaaaaggataccacttgatgagctttcc
ccagtctgccccgcacggtgtggtcttcctgcacgtaacctacgtgccggcgcaagaaaaaaactttaccaccgctcccgcgatat
gtcacgacggaaaggcgcattttccgcgagaaggcgtatttgtatcaaacggaacacactggttcgttacccagcgaaacttctac
gagccacagatcattactacggataatacattcgtctctggtaattgcgatgtagtcataggaatagtaaataatacggtttatgacc
ccctccaacctgagcttgactccttcaaagaggaacttgataaatactttaagaatcatacctctccggatgtcgatctcggggatat
ttctggtatcaatgcaagcgtcgtgaacatacagaaagaaatcgacagattgaacgaagtcgcgaagaatcttaacgaatctctg
atagacttgcaagaattgggcaagtatgagcagtatataaaatggccatggtacatttggttgggattcatcgcaggattgatcgcc
attgttatggttacaatcatgctctgttgtatgaccagttgctgtagctgcttgaagggctgctgctcatgcggttcttgttgcggcggc
cgctacccatacgacgtaccagattacgcttaaagatctttttccctctgccaaaaattatggggacatcatgaagccccttgagcatctgactt
ctggctaataaaggaaatttattttcattgcaatagtgtgttggaattttttgtgtctctcactcggaaggacatatgggagggcaaatcatttaaa
acatcagaatgagtatttggtttagagtttggcaacatatgcccattcttccgcttcctcgctcactgactcgctgcgctcggtcgttcggctgc
ggcgagcggtatcagctcactcaaaggcggtaatacggttatccacagaatcaggggataacgcaggaaagaacatgtgagcaaaagg
ccagcaaaaggccaggaaccgtaaaaaggccgcgttgctggcgtttttccataggctccgcccccctgacgagcatcacaaaaatcgac
gctcaagtcagaggtggcgaaacccgacaggactataaagataccaggcgtttccccctggaagctccctcgtgcgctctcctgttccgac
cctgccgcttaccggatacctgtccgcctttctcccttcgggaagcgtggcgctttctcatagctcacgctgtaggtatctcagttcggtgtag
gtcgttcgctccaagctgggctgtgtgcacgaaccccccgttcagcccgaccgctgcgccttatccggtaactatcgtcttgagtccaaccc
ggtaagacacgacttatcgccactggcagcagccactggtaacaggattagcagagcgaggtatgtaggcggtgctacagagttcttgaa
gtggtggcctaactacggctacactagaagaacagtatttggtatctgcgctctgctgaagccagttaccttcggaaaaagagttggtagctc
ttgatccggcaaacaaaccaccgctggtagcggtggtttttttgtttgcaagcagcagattacgcgcagaaaaaaaggatctcaagaagatc
ctttgatcttttctacggggtctgacgctcagtggaacgaaaactcacgttaagggattttggtcatgagattatcaaaaaggatcttcacctag
atccttttaaattaaaaatgaagttttaaatcaatctaaagtatatatgagtaaacttggtctgacagttaccaatgcttaatcagtgaggcacctat
ctcagcgatctgtctatttcgttcatccatagttgcctgactcggggggggggggcgctgaggtctgcctcgtgaagaaggtgttgctgactc
ataccaggcctgaatcgccccatcatccagccagaaagtgagggagccacggttgatgagagctttgttgtaggtggaccagttggtgattt
tgaacttttgctttgccacggaacggtctgcgttgtcgggaagatgcgtgatctgatccttcaactcagcaaaagttcgatttattcaacaaagc
cgccgtcccgtcaagtcagcgtaatgctctgccagtgttacaaccaattaaccaattctgattagaaaaactcatcgagcatcaaatgaaact
gcaatttattcatatcaggattatcaataccatatttttgaaaaagccgtttctgtaatgaaggagaaaactcaccgaggcagttccataggatg
gcaagatcctggtatcggtctgcgattccgactcgtccaacatcaatacaacctattaatttcccctcgtcaaaaataaggttatcaagtgaga
aatcaccatgagtgacgactgaatccggtgagaatggcaaaagcttatgcatttctttccagacttgttcaacaggccagccattacgctcgt
catcaaaatcactcgcatcaaccaaaccgttattcattcgtgattgcgcctgagcgagacgaaatacgcgatcgctgttaaaaggacaattac
aaacaggaatcgaatgcaaccggcgcaggaacactgccagcgcatcaacaatattttcacctgaatcaggatattcttctaatacctggaat
gctgttttcccggggatcgcagtggtgagtaaccatgcatcatcaggagtacggataaaatgcttgatggtcggaagaggcataaattccgt
cagccagtttagtctgaccatctcatctgtaacatcattggcaacgctacctttgccatgtttcagaaacaactctggcgcatcgggcttcccat
acaatcgatagattgtcgcacctgattgcccgacattatcgcgagcccatttatacccatataaatcagcatccatgttggaatttaatcgcggc
ctggagcaagacgtttcccgttgaatatggctcataacaccccttgtattactgtttatgtaagcagacagttttattgttcatgatgatatattttta
tcttgtgcaatgtaacatcagagattttgagacacaacgtggctttccccccccccccattattgaagcatttatcagggttattgtctcatgagc
ggatacatatttgaatgtatttagaaaaataaacaaataggggttccgcgcacatttccccgaaaagtgccacctgacgtctaagaaaccatt
attatcatgacattaacctataaaaataggcgtatcacgaggccctttcgtctcgcgcgtttcggtgatgacggtgaaaacctctgacacatgc
agctcccggagacggtcacagcttgtctgtaagcggatgccgggagcagacaagcccgtcagggcgcgtcagcgggtgttggcgggtg
tcggggctggcttaactatgcggcatcagagcagattgtactgagagtgcaccatatgcggtgtgaaataccgcacagatgcgtaaggag
aaaataccgcatcagattggctat
SEQ ID NO: 2 = amino acid sequence of the construct encoded by pSARS-CoV-2-FL.
The underlined portion shows the amino acid sequence of a modified SARS-CoV-2 Spike
protein.
MCCTKSLLLAALMSVLLLHLCGESEASVNLTTRTQLPPAYTNSFTRGVYYPDKVFRS
SVLHSTQDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWMESEF
RVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNERVQPTESIV
RFPNITNLCPFGEVENATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIP
IGAGICASYQTQTNSPRRARSVASQSIIAYTMSLGAENSVAYSNNSIAIPTNFTISVTT
EILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYG
DCLGDIAARDLICAQKENGLTVLPPLLTDEMIAQYTSALLAGTITSGWTFGAGAAL
QIPFAMQMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGRLQSLQTY
VTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGLIAIVMVTI
MLCCMTSCCSCLKGCCSCGSCCGGRYPYDVPDYA
SEQ ID NO: 3 = pMIP3α-SARS-CoV-2-FL plasmid DNA sequence. The underlined
portion shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcaactttgactgctgtcttggatacacagaccgtattcttcatcctaaatttattgtgggcttcaca
cggcagctggccaatgaaggctgtgacatcaatgctatcatctttcacacaaagaaaaagttgtctgtgtgcgcaaatccaaaacagacttg
ggtgaaatatattgtgcgtctcctcagtaaaaaagtcaagaacatggaattcaacgacgctcaggcgccgaagagtctcgaggtgaatttg
accaccaggacccagcttccgcctgcctataccaattcatttaccagaggtgtatattacccggataaagtgttccgatcctcagttc
tccattctacacaagacttgttcctccccttcttctcaaatgtgacttggtttcacgcaatccatgtgtctggaacgaatggcaccaag
aggtttgacaatccagtcttgccttttaatgatggtgtctatttcgctagcacggaaaaaagcaatataatacggggttggatcttcg
gcactacacttgacagtaaaacgcagagccttcttatagtgaataacgctaccaacgtggtaataaaagtgtgtgagttccaattct
gcaatgatccattcttgggcgtgtattaccataaaaataacaaaagttggatggaaagcgaattccgcgtgtacagctctgccaac
aactgcacttttgagtatgtgagccagccatttttgatggatctggaaggaaagcaaggtaactttaaaaacttgcgggagttcgttt
ttaagaacatcgatgggtatttcaaaatctactcaaaacacactcctattaatttggttcgcgatttgccacagggtttctctgccctc
gaacctcttgttgatctccctattggaataaatatcacgagattccagacattgctcgccctgcatcgctcatatctgacacctggtga
tagcagcagcggatggacggcaggcgcagccgcgtattacgttggctacttgcaacccagaacatttctccttaagtataatgaga
atggtacgatcactgatgcggtcgactgcgcgttggaccctctgagcgagacaaaatgcactctcaagagtttcactgtcgagaaa
ggcatttaccaaacatctaatttccgagtgcaaccaactgagtccattgttcgattcccaaacattactaacttgtgtcctttcggcga
agtgttcaacgcaacgagatttgcaagtgtatacgcgtggaaccgaaagagaatcagcaactgtgtagccgattattctgttctgta
caatagcgctagcttcagcactttcaaatgctacggtgtaagtcctacaaagctgaatgacctctgtttcacaaatgtgtacgcggat
agcttcgtcattaggggagatgaagtacggcaaatcgcccccggtcaaacagggaagatagctgactacaactataaacttccgg
atgattttacggggtgtgttatagcgtggaattctaacaacctcgattcaaaggtcgggggaaattacaattacctttaccgcctgtt
ccgaaagtcaaatcttaagccattcgaaagggacatatcaacggaaatttatcaagccgggtctactccatgcaatggcgtcgag
ggttttaattgctattttccactgcaatcttacggtttccaaccaacgaacggagtaggatatcaaccttaccgggttgtagtgttgtct
ttcgaactgctccacgcgccagctaccgtgtgcggtccgaaaaaaagcacaaatctggttaagaacaagtgtgttaacttcaacttt
aacggtctcactggaacaggtgtacttacagaatccaataagaaatttcttccgttccaacagttcggtagagacattgcggacacc
actgatgctgtgcgagatcctcagacactcgaaatactggacattacaccttgcagcttcggcggtgttagtgtaatcacgccgggc
actaatacatccaatcaggtcgctgtgttgtaccaagatgttaactgtacggaggtacctgttgcaatacatgcggaccaactcact
cctacttggagggtatattctaccggatccaacgtgttccagactcggggggctgcctgataggtgccgagcatgtcaataattctt
acgaatgcgatattccaataggggccggcatatgtgcatcttatcaaacccaaaccaactccccccgacgcgcccgctctgttgcgt
cacagagtattatagcctatacgatgtctttgggggcggaaaactccgtcgcttactccaacaacagtatagcaatccctacgaatt
ttaccatctccgtaacaactgaaatccttcctgtgtcaatgaccaaaacttctgtcgattgcacgatgtatatctgcggagacagcac
ggaatgtagtaacttgctcctccagtacggctctttctgcacacagctcaatcgggcgctgaccgggatagcggtggaacaagaca
agaacacccaggaagtcttcgcgcaggtcaagcaaatctacaagacccctcccattaaagattttggtggttttaactttagccaaa
tactgccagatccgtccaaaccctccaaacgatccttcatcgaggacctcctttttaacaaagttaccctcgccgacgccgggttcat
aaaacagtacggcgactgcttgggtgacatcgcagccagagacttgatttgtgcgcaaaaattcaacggccttacggtcctgcctc
cattgctgaccgatgaaatgattgcccagtatacctctgctttgttggcagggacaataacttctgggtggacgtttggagcaggcg
ctgccctgcagatcccttttgccatgcagatggcataccggtttaacgggatcggtgttactcagaacgtactctatgagaatcaaa
agctgatcgccaaccaatttaatagcgccatcggtaaaatccaggatagtttgagcagcaccgcttctgcacttgggaagcttcag
gatgttgtgaatcagaacgcccaagcattgaatactctcgtcaaacaattgagtagtaatttcggagccatctcatctgtgttgaatg
acatactcagtcgcctcgataaggtagaggctgaggtgcagatagaccggcttataactggtagattgcagagccttcagacttac
gtgacgcaacagcttatccgagcagcagaaatcagggcatcagcaaatcttgccgccaccaaaatgtcagagtgcgtactgggcc
aaagtaaaagagtggatttttgtggaaaaggataccacttgatgagctttccccagtctgccccgcacggtgtggtcttcctgcacg
taacctacgtgccggcgcaagaaaaaaactttaccaccgctcccgcgatatgtcacgacggaaaggcgcattttccgcgagaagg
cgtatttgtatcaaacggaacacactggttcgttacccagcgaaacttctacgagccacagatcattactacggataatacattcgt
ctctggtaattgcgatgtagtcataggaatagtaaataatacggtttatgaccccctccaacctgagcttgactccttcaaagagga
acttgataaatactttaagaatcatacctctccggatgtcgatctcggggatatttctggtatcaatgcaagcgtcgtgaacatacag
aaagaaatcgacagattgaacgaagtcgcgaagaatcttaacgaatctctgatagacttgcaagaattgggcaagtatgagcagt
atataaaatggccatggtacatttggttgggattcatcgcaggattgatcgccattgttatggttacaatcatgctctgttgtatgacc
agttgctgtagctgcttgaagggctgctgctcatgcggttcttgttgcggcggccgctacccatacgacgtaccagattacgcttaaaga
tctttttccctctgccaaaaattatggggacatcatgaagccccttgagcatctgacttctggctaataaaggaaatttattttcattgcaatagtgt
gttggaattttttgtgtctctcactcggaaggacatatgggagggcaaatcatttaaaacatcagaatgagtatttggtttagagtttggcaacat
atgcccattcttccgcttcctcgctcactgactcgctgcgctcggtcgttcggctgcggcgagcggtatcagctcactcaaaggcggtaatac
ggttatccacagaatcaggggataacgcaggaaagaacatgtgagcaaaaggccagcaaaaggccaggaaccgtaaaaaggccgcgt
tgctggcgtttttccataggctccgcccccctgacgagcatcacaaaaatcgacgctcaagtcagaggtggcgaaacccgacaggactata
aagataccaggcgtttccccctggaagctccctcgtgcgctctcctgttccgaccctgccgcttaccggatacctgtccgcctttctcccttcg
ggaagcgtggcgctttctcatagctcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgacttatcgccactggcagcagccact
ggtaacaggattagcagagcgaggtatgtaggcggtgctacagagttcttgaagtggtggcctaactacggctacactagaagaacagtat
ttggtatctgcgctctgctgaagccagttaccttcggaaaaagagttggtagctcttgatccggcaaacaaaccaccgctggtagcggtggtt
tttttgtttgcaagcagcagattacgcgcagaaaaaaaggatctcaagaagatcctttgatcttttctacggggtctgacgctcagtggaacga
aaactcacgttaagggattttggtcatgagattatcaaaaaggatcttcacctagatccttttaaattaaaaatgaagttttaaatcaatctaaagt
atatatgagtaaacttggtctgacagttaccaatgcttaatcagtgaggcacctatctcagcgatctgtctatttcgttcatccatagttgcctgac
tcggggggggggggcgctgaggtctgcctcgtgaagaaggtgttgctgactcataccaggcctgaatcgccccatcatccagccagaaa
gtgagggagccacggttgatgagagctttgttgtaggtggaccagttggtgattttgaacttttgctttgccacggaacggtctgcgttgtcgg
gaagatgcgtgatctgatccttcaactcagcaaaagttcgatttattcaacaaagccgccgtcccgtcaagtcagcgtaatgctctgccagtg
ttacaaccaattaaccaattctgattagaaaaactcatcgagcatcaaatgaaactgcaatttattcatatcaggattatcaataccatatttttga
aaaagccgtttctgtaatgaaggagaaaactcaccgaggcagttccataggatggcaagatcctggtatcggtctgcgattccgactcgtcc
aacatcaatacaacctattaatttcccctcgtcaaaaataaggttatcaagtgagaaatcaccatgagtgacgactgaatccggtgagaatgg
caaaagcttatgcatttctttccagacttgttcaacaggccagccattacgctcgtcatcaaaatcactcgcatcaaccaaaccgttattcattcg
tgattgcgcctgagcgagacgaaatacgcgatcgctgttaaaaggacaattacaaacaggaatcgaatgcaaccggcgcaggaacactg
ccagcgcatcaacaatattttcacctgaatcaggatattcttctaatacctggaatgctgttttcccggggatcgcagtggtgagtaaccatgca
tcatcaggagtacggataaaatgcttgatggtcggaagaggcataaattccgtcagccagtttagtctgaccatctcatctgtaacatcattgg
caacgctacctttgccatgtttcagaaacaactctggcgcatcgggcttcccatacaatcgatagattgtcgcacctgattgcccgacattatc
gcgagcccatttatacccatataaatcagcatccatgttggaatttaatcgcggcctggagcaagacgtttcccgttgaatatggctcataaca
ccccttgtattactgtttatgtaagcagacagttttattgttcatgatgatatatttttatcttgtgcaatgtaacatcagagattttgagacacaacgt
ggctttccccccccccccattattgaagcatttatcagggttattgtctcatgagcggatacatatttgaatgtatttagaaaaataaacaaatag
gggttccgcgcacatttccccgaaaagtgccacctgacgtctaagaaaccattattatcatgacattaacctataaaaataggcgtatcacga
ggccctttcgtctcgcgcgtttcggtgatgacggtgaaaacctctgacacatgcagctcccggagacggtcacagcttgtctgtaagcggat
gccgggagcagacaagcccgtcagggcgcgtcagcgggtgttggcgggtgtcggggctggcttaactatgcggcatcagagcagattg
tactgagagtgcaccatatgcggtgtgaaataccgcacagatgcgtaaggagaaaataccgcatcagattggctat
SEQ ID NO: 4 = Amino acid sequence of the protein encoded by pMIP3α-SARS-CoV-
2-FL. The underlined portion shows the amino acid sequence of a modified SARS-CoV-2 Spike
protein.
MCCTKSLLLAALMSVLLLHLCGESEASNFDCCLGYTDRILHPKFIVGFTRQLANEGCDI
NAIIFHTKKKLSVCANPKQTWVKYIVRLLSKKVKNMEFNDAQAPKSLEVNLTTRTQLP
PAYTNSFTRGVYYPDKVFRSSVLHSTQDLFLPFFSNVTWFHAIHVSGTNGTKRFDN
PVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTV
EKGIYQTSNERVQPTESIVRFPNITNLCPFGEVENATRFASVYAWNRKRISNCVADY
SVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARSVASQSIIAYTMSLGAEN
SVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIE
DLLFNKVTLADAGFIKQYGDCLGDIAARDLICAQKENGLTVLPPLLTDEMIAQYTS
ALLAGTITSGWTFGAGAALQIPFAMQMAYRENGIGVTQNVLYENQKLIANQFNSAI
GKIQDSLSSTASALGKLQDVVNQNAQALNTLVKQLSSNFGAISSVLNDILSRLDKVE
AEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCGGRYPYDVPDYA
SEQ ID NO: 5 = pSARS-CoV-2-ΔT plasmid DNA Sequence. The underlined portion
of SEQ ID NO: 5 shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcgtgaatttgaccaccaggacccagcttccgcctgcctataccaattcatttaccagagg
tgtatattacccggataaagtgttccgatcctcagttctccattctacacaagacttgttcctccccttcttctcaaatgtgacttggttt
cacgcaatccatgtgtctggaacgaatggcaccaagaggtttgacaatccagtcttgccttttaatgatggtgtctatttcgctagca
cggaaaaaagcaatataatacggggttggatcttcggcactacacttgacagtaaaacgcagagccttcttatagtgaataacgct
accaacgtggtaataaaagtgtgtgagttccaattctgcaatgatccattcttgggcgtgtattaccataaaaataacaaaagttgg
atggaaagcgaattccgcgtgtacagctctgccaacaactgcacttttgagtatgtgagccagccatttttgatggatctggaagga
aagcaaggtaactttaaaaacttgcgggagttcgtttttaagaacatcgatgggtatttcaaaatctactcaaaacacactcctatta
atttggttcgcgatttgccacagggtttctctgccctcgaacctcttgttgatctccctattggaataaatatcacgagattccagacat
tgctcgccctgcatcgctcatatctgacacctggtgatagcagcagcggatggacggcaggcgcagccgcgtattacgttggctac
ttgcaacccagaacatttctccttaagtataatgagaatggtacgatcactgatgcggtcgactgcgcgttggaccctctgagcgag
acaaaatgcactctcaagagtttcactgtcgagaaaggcatttaccaaacatctaatttccgagtgcaaccaactgagtccattgtt
cgattcccaaacattactaacttgtgtcctttcggcgaagtgttcaacgcaacgagatttgcaagtgtatacgcgtggaaccgaaag
agaatcagcaactgtgtagccgattattctgttctgtacaatagcgctagcttcagcactttcaaatgctacggtgtaagtcctacaa
agctgaatgacctctgtttcacaaatgtgtacgcggatagcttcgtcattaggggagatgaagtacggcaaatcgcccccggtcaa
acagggaagatagctgactacaactataaacttccggatgattttacggggtgtgttatagcgtggaattctaacaacctcgattca
aaggtcgggggaaattacaattacctttaccgcctgttccgaaagtcaaatcttaagccattcgaaagggacatatcaacggaaat
ttatcaagccgggtctactccatgcaatggcgtcgagggttttaattgctattttccactgcaatcttacggtttccaaccaacgaacg
gagtaggatatcaaccttaccgggttgtagtgttgtctttcgaactgctccacgcgccagctaccgtgtgcggtccgaaaaaaagca
caaatctggttaagaacaagtgtgttaacttcaactttaacggtctcactggaacaggtgtacttacagaatccaataagaaatttct
tccgttccaacagttcggtagagacattgcggacaccactgatgctgtgcgagatcctcagacactcgaaatactggacattacac
cttgcagcttcggcggtgttagtgtaatcacgccgggcactaatacatccaatcaggtcgctgtgttgtaccaagatgttaactgtac
ggaggtacctgttgcaatacatgcggaccaactcactcctacttggagggtatattctaccggatccaacgtgttccagactcgggc
gggctgcctgataggtgccgagcatgtcaataattcttacgaatgcgatattccaataggggccggcatatgtgcatcttatcaaac
ccaaaccaactccccccgacgcgcccgctctgttgcgtcacagagtattatagcctatacgatgtctttgggggcggaaaactccgt
cgcttactccaacaacagtatagcaatccctacgaattttaccatctccgtaacaactgaaatccttcctgtgtcaatgaccaaaact
tctgtcgattgcacgatgtatatctgcggagacagcacggaatgtagtaacttgctcctccagtacggctctttctgcacacagctca
atcgggcgctgaccgggatagcggtggaacaagacaagaacacccaggaagtcttcgcgcaggtcaagcaaatctacaagacc
cctcccattaaagattttggtggttttaactttagccaaatactgccagatccgtccaaaccctccaaacgatccttcatcgaggacct
cctttttaacaaagttaccctcgccgacgccgggttcataaaacagtacggcgactgcttgggtgacatcgcagccagagacttgat
ttgtgcgcaaaaattcaacggccttacggtcctgcctccattgctgaccgatgaaatgattgcccagtatacctctgctttgttggcag
ggacaataacttctgggtggacgtttggagcaggcgctgccctgcagatcccttttgccatgcagatggcataccggtttaacggga
tcggtgttactcagaacgtactctatgagaatcaaaagctgatcgccaaccaatttaatagcgccatcggtaaaatccaggatagtt
tgagcagcaccgcttctgcacttgggaagcttcaggatgttgtgaatcagaacgcccaagcattgaatactctcgtcaaacaattga
gtagtaatttcggagccatctcatctgtgttgaatgacatactcagtcgcctcgataaggtagaggctgaggtgcagatagaccggc
ttataactggtagattgcagagccttcagacttacgtgacgcaacagcttatccgagcagcagaaatcagggcatcagcaaatctt
gccgccaccaaaatgtcagagtgcgtactgggccaaagtaaaagagtggatttttgtggaaaaggataccacttgatgagctttcc
ccagtctgccccgcacggtgtggtcttcctgcacgtaacctacgtgccggcgcaagaaaaaaactttaccaccgctcccgcgatat
gtcacgacggaaaggcgcattttccgcgagaaggcgtatttgtatcaaacggaacacactggttcgttacccagcgaaacttctac
gagccacagatcattactacggataatacattcgtctctggtaattgcgatgtagtcataggaatagtaaataatacggtttatgacc
ccctccaacctgagcttgactccttcaaagaggaacttgataaatactttaagaatcatacctctccggatgtcgatctcggggatat
ttctggtatcaatgcaagcgtcgtgaacatacagaaagaaatcgacagattgaacgaagtcgcgaagaatcttaacgaatctctg
atagacttgcaagaattgggcaagtatgagcagggcggccgctacccatacgacgtaccagattacgcttaaagatctttttccctctgc
caaaaattatggggacatcatgaagccccttgagcatctgacttctggctaataaaggaaatttattttcattgcaatagtgtgttggaattttttgt
gtctctcactcggaaggacatatgggagggcaaatcatttaaaacatcagaatgagtatttggtttagagtttggcaacatatgcccattcttcc
gcttcctcgctcactgactcgctgcgctcggtcgttcggctgcggcgagcggtatcagctcactcaaaggcggtaatacggttatccacaga
atcaggggataacgcaggaaagaacatgtgagcaaaaggccagcaaaaggccaggaaccgtaaaaaggccgcgttgctggcgtttttc
cataggctccgcccccctgacgagcatcacaaaaatcgacgctcaagtcagaggtggcgaaacccgacaggactataaagataccagg
cgtttccccctggaagctccctcgtgcgctctcctgttccgaccctgccgcttaccggatacctgtccgcctttctcccttcgggaagcgtggc
gctttctcatagctcacgctgtaggtatctcagttcggtgtaggtcgttcgctccaagctgggctgtgtgcacgaaccccccgttcagcccga
ccgctgcgccttatccggtaactatcgtcttgagtccaacccggtaagacacgacttatcgccactggcagcagccactggtaacaggatta
gcagagcgaggtatgtaggcggtgctacagagttcttgaagtggtggcctaactacggctacactagaagaacagtatttggtatctgcgct
ctgctgaagccagttaccttcggaaaaagagttggtagctcttgatccggcaaacaaaccaccgctggtagcggtggtttttttgtttgcaagc
agcagattacgcgcagaaaaaaaggatctcaagaagatcctttgatcttttctacggggtctgacgctcagtggaacgaaaactcacgttaa
gggattttggtcatgagattatcaaaaaggatcttcacctagatccttttaaattaaaaatgaagttttaaatcaatctaaagtatatatgagtaaa
cttggtctgacagttaccaatgcttaatcagtgaggcacctatctcagcgatctgtctatttcgttcatccatagttgcctgactcggggggggg
gggcgctgaggtctgcctcgtgaagaaggtgttgctgactcataccaggcctgaatcgccccatcatccagccagaaagtgagggagcc
acggttgatgagagctttgttgtaggtggaccagttggtgattttgaacttttgctttgccacggaacggtctgcgttgtcgggaagatgcgtg
atctgatccttcaactcagcaaaagttcgatttattcaacaaagccgccgtcccgtcaagtcagcgtaatgctctgccagtgttacaaccaatt
aaccaattctgattagaaaaactcatcgagcatcaaatgaaactgcaatttattcatatcaggattatcaataccatatttttgaaaaagccgtttc
tgtaatgaaggagaaaactcaccgaggcagttccataggatggcaagatcctggtatcggtctgcgattccgactcgtccaacatcaataca
acctattaatttcccctcgtcaaaaataaggttatcaagtgagaaatcaccatgagtgacgactgaatccggtgagaatggcaaaagcttatg
catttctttccagacttgttcaacaggccagccattacgctcgtcatcaaaatcactcgcatcaaccaaaccgttattcattcgtgattgcgcctg
agcgagacgaaatacgcgatcgctgttaaaaggacaattacaaacaggaatcgaatgcaaccggcgcaggaacactgccagcgcatca
acaatattttcacctgaatcaggatattcttctaatacctggaatgctgttttcccggggatcgcagtggtgagtaaccatgcatcatcaggagt
acggataaaatgcttgatggtcggaagaggcataaattccgtcagccagtttagtctgaccatctcatctgtaacatcattggcaacgctacct
ttgccatgtttcagaaacaactctggcgcatcgggcttcccatacaatcgatagattgtcgcacctgattgcccgacattatcgcgagcccatt
tatacccatataaatcagcatccatgttggaatttaatcgcggcctggagcaagacgtttcccgttgaatatggctcataacaccccttgtatta
ctgtttatgtaagcagacagttttattgttcatgatgatatatttttatcttgtgcaatgtaacatcagagattttgagacacaacgtggctttccccc
cccccccattattgaagcatttatcagggttattgtctcatgagcggatacatatttgaatgtatttagaaaaataaacaaataggggttccgcg
cacatttccccgaaaagtgccacctgacgtctaagaaaccattattatcatgacattaacctataaaaataggcgtatcacgaggccctttcgt
ctcgcgcgtttcggtgatgacggtgaaaacctctgacacatgcagctcccggagacggtcacagcttgtctgtaagcggatgccgggagc
agacaagcccgtcagggcgcgtcagcgggtgttggcgggtgtcggggctggcttaactatgcggcatcagagcagattgtactgagagt
gcaccatatgcggtgtgaaataccgcacagatgcgtaaggagaaaataccgcatcagattggctat
SEQ ID NO: 6 = Amino acid sequence of the protein encoded by pSARS-CoV-2-ΔT.
The underlined portion shows the amino acid sequence of a modified SARS-CoV-2 Spike
protein.
MCCTKSLLLAALMSVLLLHLCGESEASVNLTTRTQLPPAYTNSFTRGVYYPDKVFRS
SVLHSTQDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWMESEF
RVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQPTESIV
RFPNITNLCPFGEVENATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIP
IGAGICASYQTQTNSPRRARSVASQSIIAYTMSLGAENSVAYSNNSIAIPTNFTISVTT
EILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYG
DCLGDIAARDLICAQKFNGLTVLPPLLTDEMIAQYTSALLAGTITSGWTFGAGAAL
QIPFAMQMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGRLQSLQTY
VTQQLIRAAEIRASANLAATKMSECVLGQSKRVDFCGKGYHLMSFPQSAPHGVVE
LHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINAS
VVNIQKEIDRLNEVAKNLNESLIDLQELGKYEQGGRYPYDVPDYA
SEQ ID NO: 7 = pMIP3α-SARS-CoV-2-ΔT Plasmid DNA sequence. The underlined
portion shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcaactttgactgctgtcttggatacacagaccgtattcttcatcctaaatttattgtgggcttcaca
cggcagctggccaatgaaggctgtgacatcaatgctatcatctttcacacaaagaaaaagttgtctgtgtgcgcaaatccaaaacagacttg
ggtgaaatatattgtgcgtctcctcagtaaaaaagtcaagaacatggaattcaacgacgctcaggcgccgaagagtctcgaggtgaatttg
accaccaggacccagcttccgcctgcctataccaattcatttaccagaggtgtatattacccggataaagtgttccgatcctcagttc
tccattctacacaagacttgttcctccccttcttctcaaatgtgacttggtttcacgcaatccatgtgtctggaacgaatggcaccaag
aggtttgacaatccagtcttgccttttaatgatggtgtctatttcgctagcacggaaaaaagcaatataatacggggttggatcttcg
gcactacacttgacagtaaaacgcagagccttcttatagtgaataacgctaccaacgtggtaataaaagtgtgtgagttccaattct
gcaatgatccattcttgggcgtgtattaccataaaaataacaaaagttggatggaaagcgaattccgcgtgtacagctctgccaac
aactgcacttttgagtatgtgagccagccatttttgatggatctggaaggaaagcaaggtaactttaaaaacttgcgggagttcgttt
ttaagaacatcgatgggtatttcaaaatctactcaaaacacactcctattaatttggttcgcgatttgccacagggtttctctgccctc
gaacctcttgttgatctccctattggaataaatatcacgagattccagacattgctcgccctgcatcgctcatatctgacacctggtga
tagcagcagcggatggacggcaggcgcagccgcgtattacgttggctacttgcaacccagaacatttctccttaagtataatgaga
atggtacgatcactgatgcggtcgactgcgcgttggaccctctgagcgagacaaaatgcactctcaagagtttcactgtcgagaaa
ggcatttaccaaacatctaatttccgagtgcaaccaactgagtccattgttcgattcccaaacattactaacttgtgtcctttcggcga
agtgttcaacgcaacgagatttgcaagtgtatacgcgtggaaccgaaagagaatcagcaactgtgtagccgattattctgttctgta
caatagcgctagcttcagcactttcaaatgctacggtgtaagtcctacaaagctgaatgacctctgtttcacaaatgtgtacgcggat
agcttcgtcattaggggagatgaagtacggcaaatcgcccccggtcaaacagggaagatagctgactacaactataaacttccgg
atgattttacggggtgtgttatagcgtggaattctaacaacctcgattcaaaggtcgggggaaattacaattacctttaccgcctgtt
ccgaaagtcaaatcttaagccattcgaaagggacatatcaacggaaatttatcaagccgggtctactccatgcaatggcgtcgag
ggttttaattgctattttccactgcaatcttacggtttccaaccaacgaacggagtaggatatcaaccttaccgggttgtagtgttgtct
ttcgaactgctccacgcgccagctaccgtgtgcggtccgaaaaaaagcacaaatctggttaagaacaagtgtgttaacttcaacttt
aacggtctcactggaacaggtgtacttacagaatccaataagaaatttcttccgttccaacagttcggtagagacattgcggacacc
actgatgctgtgcgagatcctcagacactcgaaatactggacattacaccttgcagcttcggcggtgttagtgtaatcacgccgggc
actaatacatccaatcaggtcgctgtgttgtaccaagatgttaactgtacggaggtacctgttgcaatacatgcggaccaactcact
cctacttggagggtatattctaccggatccaacgtgttccagactcggggggctgcctgataggtgccgagcatgtcaataattctt
acgaatgcgatattccaataggggccggcatatgtgcatcttatcaaacccaaaccaactccccccgacgcgcccgctctgttgcgt
cacagagtattatagcctatacgatgtctttgggggcggaaaactccgtcgcttactccaacaacagtatagcaatccctacgaatt
ttaccatctccgtaacaactgaaatccttcctgtgtcaatgaccaaaacttctgtcgattgcacgatgtatatctgcggagacagcac
ggaatgtagtaacttgctcctccagtacggctctttctgcacacagctcaatcgggcgctgaccgggatagcggtggaacaagaca
agaacacccaggaagtcttcgcgcaggtcaagcaaatctacaagacccctcccattaaagattttggtggttttaactttagccaaa
tactgccagatccgtccaaaccctccaaacgatccttcatcgaggacctcctttttaacaaagttaccctcgccgacgccgggttcat
aaaacagtacggcgactgcttgggtgacatcgcagccagagacttgatttgtgcgcaaaaattcaacggccttacggtcctgcctc
cattgctgaccgatgaaatgattgcccagtatacctctgctttgttggcagggacaataacttctgggtggacgtttggagcaggcg
ctgccctgcagatcccttttgccatgcagatggcataccggtttaacgggatcggtgttactcagaacgtactctatgagaatcaaa
agctgatcgccaaccaatttaatagcgccatcggtaaaatccaggatagtttgagcagcaccgcttctgcacttgggaagcttcag
gatgttgtgaatcagaacgcccaagcattgaatactctcgtcaaacaattgagtagtaatttcggagccatctcatctgtgttgaatg
acatactcagtcgcctcgataaggtagaggctgaggtgcagatagaccggcttataactggtagattgcagagccttcagacttac
gtgacgcaacagcttatccgagcagcagaaatcagggcatcagcaaatcttgccgccaccaaaatgtcagagtgcgtactgggcc
aaagtaaaagagtggatttttgtggaaaaggataccacttgatgagctttccccagtctgccccgcacggtgtggtcttcctgcacg
taacctacgtgccggcgcaagaaaaaaactttaccaccgctcccgcgatatgtcacgacggaaaggcgcattttccgcgagaagg
cgtatttgtatcaaacggaacacactggttcgttacccagcgaaacttctacgagccacagatcattactacggataatacattcgt
ctctggtaattgcgatgtagtcataggaatagtaaataatacggtttatgaccccctccaacctgagcttgactccttcaaagagga
acttgataaatactttaagaatcatacctctccggatgtcgatctcggggatatttctggtatcaatgcaagcgtcgtgaacatacag
aaagaaatcgacagattgaacgaagtcgcgaagaatcttaacgaatctctgatagacttgcaagaattgggcaagtatgagcag
ggcggccgctacccatacgacgtaccagattacgcttaaagatctttttccctctgccaaaaattatggggacatcatgaagccccttgagca
tctgacttctggctaataaaggaaatttattttcattgcaatagtgtgttggaattttttgtgtctctcactcggaaggacatatgggagggcaaat
catttaaaacatcagaatgagtatttggtttagagtttggcaacatatgcccattcttccgcttcctcgctcactgactcgctgcgctcggtcgttc
ggctgcggcgagcggtatcagctcactcaaaggcggtaatacggttatccacagaatcaggggataacgcaggaaagaacatgtgagca
aaaggccagcaaaaggccaggaaccgtaaaaaggccgcgttgctggcgtttttccataggctccgcccccctgacgagcatcacaaaaa
tcgacgctcaagtcagaggtggcgaaacccgacaggactataaagataccaggcgtttccccctggaagctccctcgtgcgctctcctgtt
ccgaccctgccgcttaccggatacctgtccgcctttctcccttcgggaagcgtggcgctttctcatagctcacgctgtaggtatctcagttcgg
tgtaggtcgttcgctccaagctgggctgtgtgcacgaaccccccgttcagcccgaccgctgcgccttatccggtaactatcgtcttgagtcca
acccggtaagacacgacttatcgccactggcagcagccactggtaacaggattagcagagcgaggtatgtaggcggtgctacagagttct
tgaagtggtggcctaactacggctacactagaagaacagtatttggtatctgcgctctgctgaagccagttaccttcggaaaaagagttggta
gctcttgatccggcaaacaaaccaccgctggtagcggtggtttttttgtttgcaagcagcagattacgcgcagaaaaaaaggatctcaagaa
gatcctttgatcttttctacggggtctgacgctcagtggaacgaaaactcacgttaagggattttggtcatgagattatcaaaaaggatcttcac
ctagatccttttaaattaaaaatgaagttttaaatcaatctaaagtatatatgagtaaacttggtctgacagttaccaatgcttaatcagtgaggca
cctatctcagcgatctgtctatttcgttcatccatagttgcctgactcggggggggggggcgctgaggtctgcctcgtgaagaaggtgttgct
gactcataccaggcctgaatcgccccatcatccagccagaaagtgagggagccacggttgatgagagctttgttgtaggtggaccagttgg
tgattttgaacttttgctttgccacggaacggtctgcgttgtcgggaagatgcgtgatctgatccttcaactcagcaaaagttcgatttattcaac
aaagccgccgtcccgtcaagtcagcgtaatgctctgccagtgttacaaccaattaaccaattctgattagaaaaactcatcgagcatcaaatg
aaactgcaatttattcatatcaggattatcaataccatatttttgaaaaagccgtttctgtaatgaaggagaaaactcaccgaggcagttccata
ggatggcaagatcctggtatcggtctgcgattccgactcgtccaacatcaatacaacctattaatttcccctcgtcaaaaataaggttatcaagt
gagaaatcaccatgagtgacgactgaatccggtgagaatggcaaaagcttatgcatttctttccagacttgttcaacaggccagccattacgc
tcgtcatcaaaatcactcgcatcaaccaaaccgttattcattcgtgattgcgcctgagcgagacgaaatacgcgatcgctgttaaaaggacaa
ttacaaacaggaatcgaatgcaaccggcgcaggaacactgccagcgcatcaacaatattttcacctgaatcaggatattcttctaatacctgg
aatgctgttttcccggggatcgcagtggtgagtaaccatgcatcatcaggagtacggataaaatgcttgatggtcggaagaggcataaattc
cgtcagccagtttagtctgaccatctcatctgtaacatcattggcaacgctacctttgccatgtttcagaaacaactctggcgcatcgggcttcc
catacaatcgatagattgtcgcacctgattgcccgacattatcgcgagcccatttatacccatataaatcagcatccatgttggaatttaatcgc
ggcctggagcaagacgtttcccgttgaatatggctcataacaccccttgtattactgtttatgtaagcagacagttttattgttcatgatgatatatt
tttatcttgtgcaatgtaacatcagagattttgagacacaacgtggctttccccccccccccattattgaagcatttatcagggttattgtctcatg
agcggatacatatttgaatgtatttagaaaaataaacaaataggggttccgcgcacatttccccgaaaagtgccacctgacgtctaagaaacc
attattatcatgacattaacctataaaaataggcgtatcacgaggccctttcgtctcgcgcgtttcggtgatgacggtgaaaacctctgacacat
gcagctcccggagacggtcacagcttgtctgtaagcggatgccgggagcagacaagcccgtcagggcgcgtcagcgggtgttggcgg
gtgtcggggctggcttaactatgcggcatcagagcagattgtactgagagtgcaccatatgcggtgtgaaataccgcacagatgcgtaagg
agaaaataccgcatcagattggctat
SEQ ID NO: 8 = Amino acid sequence of the protein encoded by pMIP3α-SARS-CoV-
2-ΔT. The underlined portion shows the amino acid sequence of a modified SARS-CoV-2 Spike
protein.
MCCTKSLLLAALMSVLLLHLCGESEASNFDCCLGYTDRILHPKFIVGFTRQLANEGCDI
NAIIFHTKKKLSVCANPKQTWVKYIVRLLSKKVKNMEFNDAQAPKSLEVNLTTRTQLP
PAYTNSFTRGVYYPDKVFRSSVLHSTQDLFLPFFSNVTWFHAIHVSGTNGTKRFDN
PVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVE
KNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTV
EKGIYQTSNFRVQPTESIVRFPNITNLCPFGEVENATRFASVYAWNRKRISNCVADY
SVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARSVASQSIIAYTMSLGAEN
SVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIE
DLLFNKVTLADAGFIKQYGDCLGDIAARDLICAQKENGLTVLPPLLTDEMIAQYTS
ALLAGTITSGWTFGAGAALQIPFAMQMAYRENGIGVTQNVLYENQKLIANQFNSAI
GKIQDSLSSTASALGKLQDVVNQNAQALNTLVKQLSSNFGAISSVLNDILSRLDKVE
AEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQGGRYP
YDVPDYA
SEQ ID NO: 9 = pSARS-CoV-2-ΔTS, plasmid DNA sequence. The underlined portion
shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcgtgaatttgaccaccaggacccagcttccgcctgcctataccaattcatttaccagagg
tgtatattacccggataaagtgttccgatcctcagttctccattctacacaagacttgttcctccccttcttctcaaatgtgacttggttt
cacgcaatccatgtgtctggaacgaatggcaccaagaggtttgacaatccagtcttgccttttaatgatggtgtctatttcgctagca
cggaaaaaagcaatataatacggggttggatcttcggcactacacttgacagtaaaacgcagagccttcttatagtgaataacgct
accaacgtggtaataaaagtgtgtgagttccaattctgcaatgatccattcttgggcgtgtattaccataaaaataacaaaagttgg
atggaaagcgaattccgcgtgtacagctctgccaacaactgcacttttgagtatgtgagccagccatttttgatggatctggaagga
aagcaaggtaactttaaaaacttgcgggagttcgtttttaagaacatcgatgggtatttcaaaatctactcaaaacacactcctatta
atttggttcgcgatttgccacagggtttctctgccctcgaacctcttgttgatctccctattggaataaatatcacgagattccagacat
tgctcgccctgcatcgctcatatctgacacctggtgatagcagcagcggatggacggcaggcgcagccgcgtattacgttggctac
ttgcaacccagaacatttctccttaagtataatgagaatggtacgatcactgatgcggtcgactgcgcgttggaccctctgagcgag
acaaaatgcactctcaagagtttcactgtcgagaaaggcatttaccaaacatctaatttccgagtgcaaccaactgagtccattgtt
cgattcccaaacattactaacttgtgtcctttcggcgaagtgttcaacgcaacgagatttgcaagtgtatacgcgtggaaccgaaag
agaatcagcaactgtgtagccgattattctgttctgtacaatagcgctagcttcagcactttcaaatgctacggtgtaagtcctacaa
agctgaatgacctctgtttcacaaatgtgtacgcggatagcttcgtcattaggggagatgaagtacggcaaatcgcccccggtcaa
acagggaagatagctgactacaactataaacttccggatgattttacggggtgtgttatagcgtggaattctaacaacctcgattca
aaggtcgggggaaattacaattacctttaccgcctgttccgaaagtcaaatcttaagccattcgaaagggacatatcaacggaaat
ttatcaagccgggtctactccatgcaatggcgtcgagggttttaattgctattttccactgcaatcttacggtttccaaccaacgaacg
gagtaggatatcaaccttaccgggttgtagtgttgtctttcgaactgctccacgcgccagctaccgtgtgcggtccgaaaaaaagca
caaatctggttaagaacaagtgtgttaacttcaactttaacggtctcactggaacaggtgtacttacagaatccaataagaaatttct
tccgttccaacagttcggtagagacattgcggacaccactgatgctgtgcgagatcctcagacactcgaaatactggacattacac
cttgcagcttcggcggtgttagtgtaatcacgccgggcactaatacatccaatcaggtcgctgtgttgtaccaagatgttaactgtac
ggaggtacctgttgcaatacatgcggaccaactcactcctacttggagggtatattctaccggatccaacgtgttccagactcgggc
gggctgcctgataggtgccgagcatgtcaataattcttacgaatgcgatattccaataggggccggcatatgtgcatcttatcaaac
ccaaaccaactccccctctgttgcgtcacagagtattatagcctatacgatgtctttgggggcggaaaactccgtcgcttactccaac
aacagtatagcaatccctacgaattttaccatctccgtaacaactgaaatccttcctgtgtcaatgaccaaaacttctgtcgattgca
cgatgtatatctgcggagacagcacggaatgtagtaacttgctcctccagtacggctctttctgcacacagctcaatcgggcgctga
ccgggatagcggtggaacaagacaagaacacccaggaagtcttcgcgcaggtcaagcaaatctacaagacccctcccattaaag
attttggtggttttaactttagccaaatactgccagatccgtccaaatccttcatcgaggacctcctttttaacaaagttaccctcgccg
acgccgggttcataaaacagtacggcgactgcttgggtgacatcgcagccagagacttgatttgtgcgcaaaaattcaacggcctt
acggtcctgcctccattgctgaccgatgaaatgattgcccagtatacctctgctttgttggcagggacaataacttctgggtggacgt
ttggagcaggcgctgccctgcagatcccttttgccatgcagatggcataccggtttaacgggatcggtgttactcagaacgtactct
atgagaatcaaaagctgatcgccaaccaatttaatagcgccatcggtaaaatccaggatagtttgagcagcaccgcttctgcactt
gggaagcttcaggatgttgtgaatcagaacgcccaagcattgaatactctcgtcaaacaattgagtagtaatttcggagccatctca
tctgtgttgaatgacatactcagtcgcctcgataaggtagaggctgaggtgcagatagaccggcttataactggtagattgcagag
ccttcagacttacgtgacgcaacagcttatccgagcagcagaaatcagggcatcagcaaatcttgccgccaccaaaatgtcagag
tgcgtactgggccaaagtaaaagagtggatttttgtggaaaaggataccacttgatgagctttccccagtctgccccgcacggtgtg
gtcttcctgcacgtaacctacgtgccggcgcaagaaaaaaactttaccaccgctcccgcgatatgtcacgacggaaaggcgcattt
tccgcgagaaggcgtatttgtatcaaacggaacacactggttcgttacccagcgaaacttctacgagccacagatcattactacgg
ataatacattcgtctctggtaattgcgatgtagtcataggaatagtaaataatacggtttatgaccccctccaacctgagcttgactc
cttcaaagaggaacttgataaatactttaagaatcatacctctccggatgtcgatctcggggatatttctggtatcaatgcaagcgtc
gtgaacatacagaaagaaatcgacagattgaacgaagtcgcgaagaatcttaacgaatctctgatagacttgcaagaattgggc
aagtatgagcagggcggccgctacccatacgacgtaccagattacgcttaaagatctttttccctctgccaaaaattatggggacatcatga
agccccttgagcatctgacttctggctaataaaggaaatttattttcattgcaatagtgtgttggaattttttgtgtctctcactcggaaggacatat
gggagggcaaatcatttaaaacatcagaatgagtatttggtttagagtttggcaacatatgcccattcttccgcttcctcgctcactgactcgct
gcgctcggtcgttcggctgcggcgagcggtatcagctcactcaaaggcggtaatacggttatccacagaatcaggggataacgcaggaa
agaacatgtgagcaaaaggccagcaaaaggccaggaaccgtaaaaaggccgcgttgctggcgtttttccataggctccgcccccctgac
gagcatcacaaaaatcgacgctcaagtcagaggtggcgaaacccgacaggactataaagataccaggcgtttccccctggaagctccctc
gtgcgctctcctgttccgaccctgccgcttaccggatacctgtccgcctttctcccttcgggaagcgtggcgctttctcatagctcacgctgta
ggtatctcagttcggtgtaggtcgttcgctccaagctgggctgtgtgcacgaaccccccgttcagcccgaccgctgcgccttatccggtaac
tatcgtcttgagtccaacccggtaagacacgacttatcgccactggcagcagccactggtaacaggattagcagagcgaggtatgtaggc
ggtgctacagagttcttgaagtggtggcctaactacggctacactagaagaacagtatttggtatctgcgctctgctgaagccagttaccttcg
gaaaaagagttggtagctcttgatccggcaaacaaaccaccgctggtagcggtggtttttttgtttgcaagcagcagattacgcgcagaaaa
aaaggatctcaagaagatcctttgatcttttctacggggtctgacgctcagtggaacgaaaactcacgttaagggattttggtcatgagattatc
aaaaaggatcttcacctagatccttttaaattaaaaatgaagttttaaatcaatctaaagtatatatgagtaaacttggtctgacagttaccaatgc
ttaatcagtgaggcacctatctcagcgatctgtctatttcgttcatccatagttgcctgactcggggggggggggcgctgaggtctgcctcgt
gaagaaggtgttgctgactcataccaggcctgaatcgccccatcatccagccagaaagtgagggagccacggttgatgagagctttgttgt
aggtggaccagttggtgattttgaacttttgctttgccacggaacggtctgcgttgtcgggaagatgcgtgatctgatccttcaactcagcaaa
agttcgatttattcaacaaagccgccgtcccgtcaagtcagcgtaatgctctgccagtgttacaaccaattaaccaattctgattagaaaaact
catcgagcatcaaatgaaactgcaatttattcatatcaggattatcaataccatatttttgaaaaagccgtttctgtaatgaaggagaaaactcac
cgaggcagttccataggatggcaagatcctggtatcggtctgcgattccgactcgtccaacatcaatacaacctattaatttcccctggtcaaa
aataaggttatcaagtgagaaatcaccatgagtgacgactgaatccggtgagaatggcaaaagcttatgcatttctttccagacttgttcaaca
ggccagccattacgctcgtcatcaaaatcactcgcatcaaccaaaccgttattcattcgtgattgcgcctgagcgagacgaaatacgcgatc
gctgttaaaaggacaattacaaacaggaatcgaatgcaaccggcgcaggaacactgccagcgcatcaacaatattttcacctgaatcagga
tattcttctaatacctggaatgctgttttcccggggatcgcagtggtgagtaaccatgcatcatcaggagtacggataaaatgcttgatggtcg
gaagaggcataaattccgtcagccagtttagtctgaccatctcatctgtaacatcattggcaacgctacctttgccatgtttcagaaacaactct
ggcgcatcgggcttcccatacaatcgatagattgtcgcacctgattgcccgacattatcgcgagcccatttatacccatataaatcagcatcc
atgttggaatttaatcgcggcctggagcaagacgtttcccgttgaatatggctcataacaccccttgtattactgtttatgtaagcagacagtttt
attgttcatgatgatatatttttatcttgtgcaatgtaacatcagagattttgagacacaacgtggctttccccccccccccattattgaagcatttat
cagggttattgtctcatgagcggatacatatttgaatgtatttagaaaaataaacaaataggggttccgcgcacatttccccgaaaagtgccac
ctgacgtctaagaaaccattattatcatgacattaacctataaaaataggcgtatcacgaggccctttcgtctcgcgcgtttcggtgatgacggt
gaaaacctctgacacatgcagctcccggagacggtcacagcttgtctgtaagcggatgccgggagcagacaagcccgtcagggcgcgt
cagcgggtgttggcgggtgtcggggctggcttaactatgcggcatcagagcagattgtactgagagtgcaccatatgcggtgtgaaatacc
gcacagatgcgtaaggagaaaataccgcatcagattggctat
SEQ ID NO: 10 = Amino acid sequence of the protein encoded by pSARS-CoV-2-ΔTS.
The underlined portion shows the amino acid sequence of a modified SARS-CoV-2 Spike
protein.
MCCTKSLLLAALMSVLLLHLCGESEASVNLTTRTQLPPAYTNSFTRGVYYPDKVERS
SVLHSTQDLFLPFFSNVTWFHAIHVSGTNGTKRFDNPVLPENDGVYFASTEKSNIIR
GWIFGTTLDSKTQSLLIVNNATNVVIKVCEFQFCNDPFLGVYYHKNNKSWMESEF
RVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNERVQPTESIV
RFPNITNLCPFGEVENATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIP
IGAGICASYQTQTNSPSVASQSIIAYTMSLGAENSVAYSNNSIAIPTNFTISVTTEILPV
SMTKTSVDCTMYICGDSTECSNLLLQYGSFCTQLNRALTGIAVEQDKNTQEVFAQ
VKQIYKTPPIKDFGGFNFSQILPDPSKSFIEDLLENKVTLADAGFIKQYGDCLGDIAA
RDLICAQKFNGLTVLPPLLTDEMIAQYTSALLAGTITSGWTFGAGAALQIPFAMQM
AYRENGIGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDR
LNEVAKNLNESLIDLQELGKYEQGGRYPYDVPDYA
SEQ ID NO: 11 = pMIP3α-SARS-CoV-2-ΔTS plasmid DNA sequence. The underlined
portion shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcaactttgactgctgtcttggatacacagaccgtattcttcatcctaaatttattgtgggcttcaca
cggcagctggccaatgaaggctgtgacatcaatgctatcatctttcacacaaagaaaaagttgtctgtgtgcgcaaatccaaaacagacttg
ggtgaaatatattgtgcgtctcctcagtaaaaaagtcaagaacatggaattcaacgacgctcaggcgccgaagagtctcgaggtgaatttg
accaccaggacccagcttccgcctgcctataccaattcatttaccagaggtgtatattacccggataaagtgttccgatcctcagttc
tccattctacacaagacttgttcctccccttcttctcaaatgtgacttggtttcacgcaatccatgtgtctggaacgaatggcaccaag
aggtttgacaatccagtcttgccttttaatgatggtgtctatttcgctagcacggaaaaaagcaatataatacggggttggatcttcg
gcactacacttgacagtaaaacgcagagccttcttatagtgaataacgctaccaacgtggtaataaaagtgtgtgagttccaattct
gcaatgatccattcttgggcgtgtattaccataaaaataacaaaagttggatggaaagcgaattccgcgtgtacagctctgccaac
aactgcacttttgagtatgtgagccagccatttttgatggatctggaaggaaagcaaggtaactttaaaaacttgcgggagttcgttt
ttaagaacatcgatgggtatttcaaaatctactcaaaacacactcctattaatttggttcgcgatttgccacagggtttctctgccctc
gaacctcttgttgatctccctattggaataaatatcacgagattccagacattgctcgccctgcatcgctcatatctgacacctggtga
tagcagcagcggatggacggcaggcgcagccgcgtattacgttggctacttgcaacccagaacatttctccttaagtataatgaga
atggtacgatcactgatgcggtcgactgcgcgttggaccctctgagcgagacaaaatgcactctcaagagtttcactgtcgagaaa
ggcatttaccaaacatctaatttccgagtgcaaccaactgagtccattgttcgattcccaaacattactaacttgtgtcctttcggcga
agtgttcaacgcaacgagatttgcaagtgtatacgcgtggaaccgaaagagaatcagcaactgtgtagccgattattctgttctgta
caatagcgctagcttcagcactttcaaatgctacggtgtaagtcctacaaagctgaatgacctctgtttcacaaatgtgtacgcggat
agcttcgtcattaggggagatgaagtacggcaaatcgcccccggtcaaacagggaagatagctgactacaactataaacttccgg
atgattttacggggtgtgttatagcgtggaattctaacaacctcgattcaaaggtcgggggaaattacaattacctttaccgcctgtt
ccgaaagtcaaatcttaagccattcgaaagggacatatcaacggaaatttatcaagccgggtctactccatgcaatggcgtcgag
ggttttaattgctattttccactgcaatcttacggtttccaaccaacgaacggagtaggatatcaaccttaccgggttgtagtgttgtct
ttcgaactgctccacgcgccagctaccgtgtgcggtccgaaaaaaagcacaaatctggttaagaacaagtgtgttaacttcaacttt
aacggtctcactggaacaggtgtacttacagaatccaataagaaatttcttccgttccaacagttcggtagagacattgcggacacc
actgatgctgtgcgagatcctcagacactcgaaatactggacattacaccttgcagcttcggcggtgttagtgtaatcacgccgggc
actaatacatccaatcaggtcgctgtgttgtaccaagatgttaactgtacggaggtacctgttgcaatacatgcggaccaactcact
cctacttggagggtatattctaccggatccaacgtgttccagactcggggggctgcctgataggtgccgagcatgtcaataattctt
acgaatgcgatattccaataggggccggcatatgtgcatcttatcaaacccaaaccaactccccctctgttgcgtcacagagtatta
tagcctatacgatgtctttgggggcggaaaactccgtcgcttactccaacaacagtatagcaatccctacgaattttaccatctccgt
aacaactgaaatccttcctgtgtcaatgaccaaaacttctgtcgattgcacgatgtatatctgcggagacagcacggaatgtagtaa
cttgctcctccagtacggctctttctgcacacagctcaatcgggcgctgaccgggatagcggtggaacaagacaagaacacccag
gaagtcttcgcgcaggtcaagcaaatctacaagacccctcccattaaagattttggtggttttaactttagccaaatactgccagatc
cgtccaaatccttcatcgaggacctcctttttaacaaagttaccctcgccgacgccgggttcataaaacagtacggcgactgcttgg
gtgacatcgcagccagagacttgatttgtgcgcaaaaattcaacggccttacggtcctgcctccattgctgaccgatgaaatgattg
cccagtatacctctgctttgttggcagggacaataacttctgggtggacgtttggagcaggcgctgccctgcagatcccttttgccat
gcagatggcataccggtttaacgggatcggtgttactcagaacgtactctatgagaatcaaaagctgatcgccaaccaatttaata
gcgccatcggtaaaatccaggatagtttgagcagcaccgcttctgcacttgggaagcttcaggatgttgtgaatcagaacgcccaa
gcattgaatactctcgtcaaacaattgagtagtaatttcggagccatctcatctgtgttgaatgacatactcagtcgcctcgataagg
tagaggctgaggtgcagatagaccggcttataactggtagattgcagagccttcagacttacgtgacgcaacagcttatccgagca
gcagaaatcagggcatcagcaaatcttgccgccaccaaaatgtcagagtgcgtactgggccaaagtaaaagagtggatttttgtg
gaaaaggataccacttgatgagctttccccagtctgccccgcacggtgtggtcttcctgcacgtaacctacgtgccggcgcaagaa
aaaaactttaccaccgctcccgcgatatgtcacgacggaaaggcgcattttccgcgagaaggcgtatttgtatcaaacggaacac
actggttcgttacccagcgaaacttctacgagccacagatcattactacggataatacattcgtctctggtaattgcgatgtagtcat
aggaatagtaaataatacggtttatgaccccctccaacctgagcttgactccttcaaagaggaacttgataaatactttaagaatca
tacctctccggatgtcgatctcggggatatttctggtatcaatgcaagcgtcgtgaacatacagaaagaaatcgacagattgaacg
aagtcgcgaagaatcttaacgaatctctgatagacttgcaagaattgggcaagtatgagcagggcggccgctacccatacgacgta
ccagattacgcttaaagatctttttccctctgccaaaaattatggggacatcatgaagccccttgagcatctgacttctggctaataaaggaaat
ttattttcattgcaatagtgtgttggaattttttgtgtctctcactcggaaggacatatgggagggcaaatcatttaaaacatcagaatgagtatttg
gtttagagtttggcaacatatgcccattcttccgcttcctcgctcactgactcgctgcgctcggtcgttcggctgcggcgagcggtatcagctc
actcaaaggcggtaatacggttatccacagaatcaggggataacgcaggaaagaacatgtgagcaaaaggccagcaaaaggccaggaa
ccgtaaaaaggccgcgttgctggcgtttttccataggctccgcccccctgacgagcatcacaaaaatcgacgctcaagtcagaggtggcg
aaacccgacaggactataaagataccaggcgtttccccctggaagctccctcgtgcgctctcctgttccgaccctgccgcttaccggatacc
tgtccgcctttctcccttcgggaagcgtggcgctttctcatagctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgc
cactggcagcagccactggtaacaggattagcagagcgaggtatgtaggcggtgctacagagttcttgaagtggtggcctaactacggct
acactagaagaacagtatttggtatctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcacctagatccttttaaattaaaaatgaa
gttttaaatcaatctaaagtatatatgagtaaacttggtctgacagttaccaatgcttaatcagtgaggcacctatctcagcgatctgtctatttcgt
tcatccatagttgcctgactcggggggggggggcgctgaggtctgcctcgtgaagaaggtgttgctgactcataccaggcctgaatcgcc
ccatcatccagccagaaagtgagggagccacggttgatgagagctttgttgtaggtggaccagttggtgattttgaacttttgctttgccacg
gaacggtctgcgttgtcgggaagatgcgtgatctgatccttcaactcagcaaaagttcgatttattcaacaaagccgccgtcccgtcaagtca
gcgtaatgctctgccagtgttacaaccaattaaccaattctgattagaaaaactcatcgagcatcaaatgaaactgcaatttattcatatcagga
ttatcaataccatatttttgaaaaagccgtttctgtaatgaaggagaaaactcaccgaggcagttccataggatggcaagatcctggtatcggt
ctgcgattccgactcgtccaacatcaatacaacctattaatttcccctcgtcaaaaataaggttatcaagtgagaaatcaccatgagtgacgac
tgaatccggtgagaatggcaaaagcttatgcatttctttccagacttgttcaacaggccagccattacgctcgtcatcaaaatcactcgcatca
accaaaccgttattcattcgtgattgcgcctgagcgagacgaaatacgcgatcgctgttaaaaggacaattacaaacaggaatcgaatgcaa
ccggcgcaggaacactgccagcgcatcaacaatattttcacctgaatcaggatattcttctaatacctggaatgctgttttcccggggatcgc
agtggtgagtaaccatgcatcatcaggagtacggataaaatgcttgatggtcggaagaggcataaattccgtcagccagtttagtctgacca
tctcatctgtaacatcattggcaacgctacctttgccatgtttcagaaacaactctggcgcatcgggcttcccatacaatcgatagattgtcgca
cctgattgcccgacattatcgcgagcccatttatacccatataaatcagcatccatgttggaatttaatcgcggcctggagcaagacgtttccc
gttgaatatggctcataacaccccttgtattactgtttatgtaagcagacagttttattgttcatgatgatatatttttatcttgtgcaatgtaacatca
gagattttgagacacaacgtggctttccccccccccccattattgaagcatttatcagggttattgtctcatgagcggatacatatttgaatgtatt
tagaaaaataaacaaataggggttccgcgcacatttccccgaaaagtgccacctgacgtctaagaaaccattattatcatgacattaacctat
aaaaataggcgtatcacgaggccctttcgtctcgcgcgtttcggtgatgacggtgaaaacctctgacacatgcagctcccggagacggtca
cagcttgtctgtaagcggatgccgggagcagacaagcccgtcagggcgcgtcagcgggtgttggcgggtgtcggggctggcttaactat
gcggcatcagagcagattgtactgagagtgcaccatatgcggtgtgaaataccgcacagatgcgtaaggagaaaataccgcatcagattg
gctat
SEQ ID NO: 12 = Amino acid sequence of the protein encoded by pMIP3α-SARS-
CoV-2-ΔTS. The underlined portion shows the amino acid sequence of a modified SARS-CoV-2
Spike protein.
MCCTKSLLLAALMSVLLLHLCGESEASNFDCCLGYTDRILHPKFIVGFTRQLANEGCDI
NAIIFHTKKKLSVCANPKQTWVKYIVRLLSKKVKNMEFNDAQAPKSLEVNLTTRTQLP
PAYTNSFTRGVYYPDKVFRSSVLHSTQDLFLPFFSNVTWFHAIHVSGTNGTKRFDN
PVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTV
EKGIYQTSNERVQPTESIVRFPNITNLCPFGEVENATRFASVYAWNRKRISNCVADY
SVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYSTGSNVFQT
RAGCLIGAEHVNNSYECDIPIGAGICASYQTQTNSPSVASQSIIAYTMSLGAENSVAY
SNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKSFIEDLLENKVT
LADAGFIKQYGDCLGDIAARDLICAQKFNGLTVLPPLLTDEMIAQYTSALLAGTITS
GWTFGAGAALQIPFAMQMAYRENGIGVTQNVLYENQKLIANQFNSAIGKIQDSLSS
TASALGKLQDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKGYHLMSE
PQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDV
DLGDISGINASVVNIQKEIDRLNEVAKNLNESLIDLQELGKYEQGGRYPYDVPDYA
SEQ ID NO: 13 = pSARS-CoV-2-ΔTSL plasmid DNA sequence. The underlined
portion shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcgtgaatttgaccaccaggacccagcttccgcctgcctataccaattcatttaccagagg
tgtatattacccggataaagtgttccgatcctcagttctccattctacacaagacttgttcctccccttcttctcaaatgtgacttggttt
cacgcaatccatgtgtctggaacgaatggcaccaagaggtttgacaatccagtcttgccttttaatgatggtgtctatttcgctagca
cggaaaaaagcaatataatacggggttggatcttcggcactacacttgacagtaaaacgcagagccttcttatagtgaataacgct
accaacgtggtaataaaagtgtgtgagttccaattctgcaatgatccattcttgggcgtgtattaccataaaaataacaaaagttgg
atggaaagcgaattccgcgtgtacagctctgccaacaactgcacttttgagtatgtgagccagccatttttgatggatctggaagga
aagcaaggtaactttaaaaacttgcgggagttcgtttttaagaacatcgatgggtatttcaaaatctactcaaaacacactcctatta
atttggttcgcgatttgccacagggtttctctgccctcgaacctcttgttgatctccctattggaataaatatcacgagattccagacat
tgctcgccctgcatcgctcatatctgacacctggtgatagcagcagcggatggacggcaggcgcagccgcgtattacgttggctac
ttgcaacccagaacatttctccttaagtataatgagaatggtacgatcactgatgcggtcgactgcgcgttggaccctctgagcgag
acaaaatgcactctcaagagtttcactgtcgagaaaggcatttaccaaacatctaatttccgagtgcaaccaactgagtccattgtt
cgattcccaaacattactaacttgtgtcctttcggcgaagtgttcaacgcaacgagatttgcaagtgtatacgcgtggaaccgaaag
agaatcagcaactgtgtagccgattattctgttctgtacaatagcgctagcttcagcactttcaaatgctacggtgtaagtcctacaa
agctgaatgacctctgtttcacaaatgtgtacgcggatagcttcgtcattaggggagatgaagtacggcaaatcgcccccggtcaa
acagggaagatagctgactacaactataaacttccggatgattttacggggtgtgttatagcgtggaattctaacaacctcgattca
aaggtcgggggaaattacaattacctttaccgcctgttccgaaagtcaaatcttaagccattcgaaagggacatatcaacggaaat
ttatcaagccgggtctactccatgcaatggcgtcgagggttttaattgctattttccactgcaatcttacggtttccaaccaacgaacg
gagtaggatatcaaccttaccgggttgtagtgttgtctttcgaactgctccacgcgccagctaccgtgtgcggtccgaaaaaaagca
caaatctggttaagaacaagtgtgttaacttcaactttaacggtctcactggaacaggtgtacttacagaatccaataagaaatttct
tccgttccaacagttcggtagagacattgcggacaccactgatgctgtgcgagatcctcagacactcgaaatactggacattacac
cttgcagcttcggcggtgttagtgtaatcacgccgggcactaatacatccaatcaggtcgctgtgttgtaccaagatgttaactgtac
ggaggtacctgttgcaatacatgcggaccaactcactcctacttggagggtatattctaccggatccaacgtgttccagactcgggc
gggctgcctgataggtgccgagcatgtcaataattcttacgaatgcgatattccaataggggccggcatatgtgcatcttatcaaac
ccaaaccaactccccctctggtgggggaggctcaggcggaggtggtagcgggggcggtggttcatctgttgcgtcacagagtatta
tagcctatacgatgtctttgggggcggaaaactccgtcgcttactccaacaacagtatagcaatccctacgaattttaccatctccgt
aacaactgaaatccttcctgtgtcaatgaccaaaacttctgtcgattgcacgatgtatatctgcggagacagcacggaatgtagtaa
cttgctcctccagtacggctctttctgcacacagctcaatcgggcgctgaccgggatagcggtggaacaagacaagaacacccag
gaagtcttcgcgcaggtcaagcaaatctacaagacccctcccattaaagattttggtggttttaactttagccaaatactgccagatc
cgtccaaatctgggggtgggggaagtgggggaggggggagcggcggaggaggtagttccttcatcgaggacctcctttttaacaa
agttaccctcgccgacgccgggttcataaaacagtacggcgactgcttgggtgacatcgcagccagagacttgatttgtgcgcaaa
aattcaacggccttacggtcctgcctccattgctgaccgatgaaatgattgcccagtatacctctgctttgttggcagggacaataac
ttctgggtggacgtttggagcaggcgctgccctgcagatcccttttgccatgcagatggcataccggtttaacgggatcggtgttact
cagaacgtactctatgagaatcaaaagctgatcgccaaccaatttaatagcgccatcggtaaaatccaggatagtttgagcagca
ccgcttctgcacttgggaagcttcaggatgttgtgaatcagaacgcccaagcattgaatactctcgtcaaacaattgagtagtaattt
cggagccatctcatctgtgttgaatgacatactcagtcgcctcgataaggtagaggctgaggtgcagatagaccggcttataactg
gtagattgcagagccttcagacttacgtgacgcaacagcttatccgagcagcagaaatcagggcatcagcaaatcttgccgccac
caaaatgtcagagtgcgtactgggccaaagtaaaagagtggatttttgtggaaaaggataccacttgatgagctttccccagtctg
ccccgcacggtgtggtcttcctgcacgtaacctacgtgccggcgcaagaaaaaaactttaccaccgctcccgcgatatgtcacgac
ggaaaggcgcattttccgcgagaaggcgtatttgtatcaaacggaacacactggttcgttacccagcgaaacttctacgagccaca
gatcattactacggataatacattcgtctctggtaattgcgatgtagtcataggaatagtaaataatacggtttatgaccccctccaa
cctgagcttgactccttcaaagaggaacttgataaatactttaagaatcatacctctccggatgtcgatctcggggatatttctggtat
caatgcaagcgtcgtgaacatacagaaagaaatcgacagattgaacgaagtcgcgaagaatcttaacgaatctctgatagacttg
caagaattgggcaagtatgagcagggcggccgctacccatacgacgtaccagattacgcttaaagatctttttccctctgccaaaaattat
ggggacatcatgaagccccttgagcatctgacttctggctaataaaggaaatttattttcattgcaatagtgtgttggaattttttgtgtctctcact
cggaaggacatatgggagggcaaatcatttaaaacatcagaatgagtatttggtttagagtttggcaacatatgcccattcttccgcttcctcg
ctcactgactcgctgcgctcggtcgttcggctgcggcgagcggtatcagctcactcaaaggcggtaatacggttatccacagaatcagggg
ataacgcaggaaagaacatgtgagcaaaaggccagcaaaaggccaggaaccgtaaaaaggccgcgttgctggcgtttttccataggctc
cgcccccctgacgagcatcacaaaaatcgacgctcaagtcagaggtggcgaaacccgacaggactataaagataccaggcgtttccccc
tggaagctccctcgtgcgctctcctgttccgaccctgccgcttaccggatacctgtccgcctttctcccttcgggaagcgtggcgctttctcat
agctcacgctgtaggtatctcagttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagccactggtaacaggattagcagagc
gaggtatgtaggcggtgctacagagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtttttttgtttgcaagcagcagatt
acgcgcagaaaaaaaggatctcaagaagatcctttgatcttttctacggggtctgacgctcagtggaacgaaaactcacgttaagggattttg
gtcatgagattatcaaaaaggatcttcacctagatccttttaaattaaaaatgaagttttaaatcaatctaaagtatatatgagtaaacttggtctga
cagttaccaatgcttaatcagtgaggcacctatctcagcgatctgtctatttcgttcatccatagttgcctgactcggggggggggggcgctg
aggtctgcctcgtgaagaaggtgttgctgactcataccaggcctgaatcgccccatcatccagccagaaagtgagggagccacggttgat
gagagctttgttgtaggtggaccagttggtgattttgaacttttgctttgccacggaacggtctgcgttgtcgggaagatgcgtgatctgatcct
tcaactcagcaaaagttcgatttattcaacaaagccgccgtcccgtcaagtcagcgtaatgctctgccagtgttacaaccaattaaccaattct
gattagaaaaactcatcgagcatcaaatgaaactgcaatttattcatatcaggattatcaataccatatttttgaaaaagccgtttctgtaatgaa
ggagaaaactcaccgaggcagttccataggatggcaagatcctggtatcggtctgcgattccgactcgtccaacatcaatacaacctattaa
tttcccctcgtcaaaaataaggttatcaagtgagaaatcaccatgagtgacgactgaatccggtgagaatggcaaaagcttatgcatttctttc
cagacttgttcaacaggccagccattacgctcgtcatcaaaatcactcgcatcaaccaaaccgttattcattcgtgattgcgcctgagcgaga
cgaaatacgcgatcgctgttaaaaggacaattacaaacaggaatcgaatgcaaccggcgcaggaacactgccagcgcatcaacaatatttt
cacctgaatcaggatattcttctaatacctggaatgctgttttcccggggatcgcagtggtgagtaaccatgcatcatcaggagtacggataa
aatgcttgatggtcggaagaggcataaattccgtcagccagtttagtctgaccatctcatctgtaacatcattggcaacgctacctttgccatgt
ttcagaaacaactctggcgcatcgggcttcccatacaatcgatagattgtcgcacctgattgcccgacattatcgcgagcccatttatacccat
ataaatcagcatccatgttggaatttaatcgcggcctggagcaagacgtttcccgttgaatatggctcataacaccccttgtattactgtttatgt
aagcagacagttttattgttcatgatgatatatttttatcttgtgcaatgtaacatcagagattttgagacacaacgtggctttcccccccccccca
ttattgaagcatttatcagggttattgtctcatgagcggatacatatttgaatgtatttagaaaaataaacaaataggggttccgcgcacatttccc
cgaaaagtgccacctgacgtctaagaaaccattattatcatgacattaacctataaaaataggcgtatcacgaggccctttcgtctcgcgcgtt
tcggtgatgacggtgaaaacctctgacacatgcagctcccggagacggtcacagcttgtctgtaagcggatgccgggagcagacaagcc
cgtcagggcgcgtcagcgggtgttggcgggtgtcggggctggcttaactatgcggcatcagagcagattgtactgagagtgcaccatatg
cggtgtgaaataccgcacagatgcgtaaggagaaaataccgcatcagattggctat
SEQ ID NO: 14 = Amino acid sequence of the protein encoded by pSARS-CoV-2-
ΔTSL. The underlined portion shows the amino acid sequence of a modified SARS-CoV-2
Spike protein.
MCCTKSLLLAALMSVLLLHLCGESEASVNLTTRTQLPPAYTNSFTRGVYYPDKVFRS
SVLHSTQDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWMESEF
RVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNERVQPTESIV
RFPNITNLCPFGEVENATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGV
LTESNKKELPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIP
IGAGICASYQTQTNSPSGGGGSGGGGSGGGGSSVASQSIIAYTMSLGAENSVAYSNN
SIAIPTNFTISVTTEILPVSMTKTSVDCTMYICGDSTECSNLLLQYGSFCTQLNRALT
GIAVEQDKNTQEVFAQVKQIYKTPPIKDFGGFNFSQILPDPSKSGGGGSGGGGSGG
GGSSFIEDLLENKVTLADAGFIKQYGDCLGDIAARDLICAQKENGLTVLPPLLTDE
MIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGK
YEQGGRYPYDVPDYA
SEQ ID NO: 15 = pMIP3α-SARS-CoV-2-ΔTSL plasmid DNA sequence. The
underlined portion shows the DNA sequence coding for a modified SARS-CoV-2 Spike protein.
tggccattgcatacgttgtatccatatcataatatgtacatttatattggctcatgtccaacattaccgccatgttgacattgattattgactagttatt
aatagtaatcaattacggggtcattagttcatagcccatatatggagttccgcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggggtaggcgtgtacggtgggaggtctatataagcagagct
cgtttagtgaaccgtcagatcgcctggagacgccatccacgctgttttgacctccatagaagacaccgggaccgatccagcctccgcggc
cgggaacggtgcattggaacgcggattccccgtgccaagagtgacgtaagtaccgcctatagactctataggcacacccctttggctcttat
gcatgctatactgtttttggcttggggcctatacacccccgcttccttatgctataggtgatggtatagcttagcctataggtgtgggttattgacc
attattgaccactccaacggtggagggcagtgtagtctgagcagtactcgttgctgccgcgcgcgccaccagacataatagctgacagact
aacagactgttcctttccatgggtcttttctgcaggccgccaccatgtgctgtaccaagagtttgctcctggctgctttgatgtcagtgctgctac
tccacctctgcggcgaatcagaagcaagcaactttgactgctgtcttggatacacagaccgtattcttcatcctaaatttattgtgggcttcaca
cggcagctggccaatgaaggctgtgacatcaatgctatcatctttcacacaaagaaaaagttgtctgtgtgcgcaaatccaaaacagacttg
ggtgaaatatattgtgcgtctcctcagtaaaaaagtcaagaacatggaattcaacgacgctcaggcgccgaagagtctcgaggtgaatttg
accaccaggacccagcttccgcctgcctataccaattcatttaccagaggtgtatattacccggataaagtgttccgatcctcagttc
tccattctacacaagacttgttcctccccttcttctcaaatgtgacttggtttcacgcaatccatgtgtctggaacgaatggcaccaag
aggtttgacaatccagtcttgccttttaatgatggtgtctatttcgctagcacggaaaaaagcaatataatacggggttggatcttcg
gcactacacttgacagtaaaacgcagagccttcttatagtgaataacgctaccaacgtggtaataaaagtgtgtgagttccaattct
gcaatgatccattcttgggcgtgtattaccataaaaataacaaaagttggatggaaagcgaattccgcgtgtacagctctgccaac
aactgcacttttgagtatgtgagccagccatttttgatggatctggaaggaaagcaaggtaactttaaaaacttgcgggagttcgttt
ttaagaacatcgatgggtatttcaaaatctactcaaaacacactcctattaatttggttcgcgatttgccacagggtttctctgccctc
gaacctcttgttgatctccctattggaataaatatcacgagattccagacattgctcgccctgcatcgctcatatctgacacctggtga
tagcagcagcggatggacggcaggcgcagccgcgtattacgttggctacttgcaacccagaacatttctccttaagtataatgaga
atggtacgatcactgatgcggtcgactgcgcgttggaccctctgagcgagacaaaatgcactctcaagagtttcactgtcgagaaa
ggcatttaccaaacatctaatttccgagtgcaaccaactgagtccattgttcgattcccaaacattactaacttgtgtcctttcggcga
agtgttcaacgcaacgagatttgcaagtgtatacgcgtggaaccgaaagagaatcagcaactgtgtagccgattattctgttctgta
caatagcgctagcttcagcactttcaaatgctacggtgtaagtcctacaaagctgaatgacctctgtttcacaaatgtgtacgcggat
agcttcgtcattaggggagatgaagtacggcaaatcgcccccggtcaaacagggaagatagctgactacaactataaacttccgg
atgattttacggggtgtgttatagcgtggaattctaacaacctcgattcaaaggtcgggggaaattacaattacctttaccgcctgtt
ccgaaagtcaaatcttaagccattcgaaagggacatatcaacggaaatttatcaagccgggtctactccatgcaatggcgtcgag
ggttttaattgctattttccactgcaatcttacggtttccaaccaacgaacggagtaggatatcaaccttaccgggttgtagtgttgtct
ttcgaactgctccacgcgccagctaccgtgtgcggtccgaaaaaaagcacaaatctggttaagaacaagtgtgttaacttcaacttt
aacggtctcactggaacaggtgtacttacagaatccaataagaaatttcttccgttccaacagttcggtagagacattgcggacacc
actgatgctgtgcgagatcctcagacactcgaaatactggacattacaccttgcagcttcggcggtgttagtgtaatcacgccgggc
actaatacatccaatcaggtcgctgtgttgtaccaagatgttaactgtacggaggtacctgttgcaatacatgcggaccaactcact
cctacttggagggtatattctaccggatccaacgtgttccagactcgggcgggctgcctgataggtgccgagcatgtcaataattctt
acgaatgcgatattccaataggggccggcatatgtgcatcttatcaaacccaaaccaactccccctctggtgggggaggctcaggc
ggaggtggtagcgggggcggtggttcatctgttgcgtcacagagtattatagcctatacgatgtctttgggggcggaaaactccgtc
gcttactccaacaacagtatagcaatccctacgaattttaccatctccgtaacaactgaaatccttcctgtgtcaatgaccaaaactt
ctgtcgattgcacgatgtatatctgcggagacagcacggaatgtagtaacttgctcctccagtacggctctttctgcacacagctcaa
tcgggcgctgaccgggatagcggtggaacaagacaagaacacccaggaagtcttcgcgcaggtcaagcaaatctacaagaccc
ctcccattaaagattttggtggttttaactttagccaaatactgccagatccgtccaaatctgggggtgggggaagtgggggagggg
ggagcggcggaggaggtagttccttcatcgaggacctcctttttaacaaagttaccctcgccgacgccgggttcataaaacagtac
ggcgactgcttgggtgacatcgcagccagagacttgatttgtgcgcaaaaattcaacggccttacggtcctgcctccattgctgacc
gatgaaatgattgcccagtatacctctgctttgttggcagggacaataacttctgggtggacgtttggagcaggcgctgccctgcag
atcccttttgccatgcagatggcataccggtttaacgggatcggtgttactcagaacgtactctatgagaatcaaaagctgatcgcc
aaccaatttaatagcgccatcggtaaaatccaggatagtttgagcagcaccgcttctgcacttgggaagcttcaggatgttgtgaat
cagaacgcccaagcattgaatactctcgtcaaacaattgagtagtaatttcggagccatctcatctgtgttgaatgacatactcagtc
gcctcgataaggtagaggctgaggtgcagatagaccggcttataactggtagattgcagagccttcagacttacgtgacgcaaca
@cttatccgagcagcagaaatcagggcatcagcaaatcttgccgccaccaaaatgtcagagtgcgtactgggccaaagtaaaag
agtggatttttgtggaaaaggataccacttgatgagctttccccagtctgccccgcacggtgtggtcttcctgcacgtaacctacgtg
ccggcgcaagaaaaaaactttaccaccgctcccgcgatatgtcacgacggaaaggcgcattttccgcgagaaggcgtatttgtatc
aaacggaacacactggttcgttacccagcgaaacttctacgagccacagatcattactacggataatacattcgtctctggtaattg
cgatgtagtcataggaatagtaaataatacggtttatgaccccctccaacctgagcttgactccttcaaagaggaacttgataaata
ctttaagaatcatacctctccggatgtcgatctcggggatatttctggtatcaatgcaagcgtcgtgaacatacagaaagaaatcga
cagattaacgaagtcgcgaagaatcttaacgaatctctgatagacttgcaagaattgggcaagtatgagcagggcggccgctac
ccatacgacgtaccagattacgcttaaagatctttttccctctgccaaaaattatggggacatcatgaagccccttgagcatctgacttctggct
aataaaggaaatttattttcattgcaatagtgtgttggaattttttgtgtctctcactcggaaggacatatgggagggcaaatcatttaaaacatca
gaatgagtatttggtttagagtttggcaacatatgcccattcttccgcttcctcgctcactgactcgctgcgctcggtcgttcggctgcggcgag
cggtatcagctcactcaaaggcggtaatacggttatccacagaatcaggggataacgcaggaaagaacatgtgagcaaaaggccagcaa
aaggccaggaaccgtaaaaaggccgcgttgctggcgtttttccataggctccgcccccctgacgagcatcacaaaaatcgacgctcaagt
cagaggtggcgaaacccgacaggactataaagataccaggcgtttccccctggaagctccctcgtgcgctctcctgttccgaccctgccg
cttaccggatacctgtccgcctttctcccttcgggaagcgtggcgctttctcatagctcacgctgtaggtatctcagttcggtgtaggtcgttcg
ctccaagctgggctgtgtgcacgaaccccccgttcagcccgaccgctgcgccttatccggtaactatcgtcttgagtccaacccggtaaga
cacgacttatcgccactggcagcagccactggtaacaggattagcagagcgaggtatgtaggcggtgctacagagttcttgaagtggtgg
cctaactacggctacactagaagaacagtatttggtatctgcgctctgctgaagccagttaccttcggaaaaagagttggtagctcttgatccg
gcaaacaaaccaccgctggtagcggtggtttttttgtttgcaagcagcagattacgcgcagaaaaaaaggatctcaagaagatcctttgatct
tttctacggggtctgacgctcagtggaacgaaaactcacgttaagggattttggtcatgagattatcaaaaaggatcttcacctagatcctttta
aattaaaaatgaagttttaaatcaatctaaagtatatatgagtaaacttggtctgacagttaccaatgcttaatcagtgaggcacctatctcagcg
atctgtctatttcgttcatccatagttgcctgactcggggggggggggcgctgaggtctgcctcgtgaagaaggtgttgctgactcataccag
gcctgaatcgccccatcatccagccagaaagtgagggagccacggttgatgagagctttgttgtaggtggaccagttggtgattttgaacttt
tgctttgccacggaacggtctgcgttgtcgggaagatgcgtgatctgatccttcaactcagcaaaagttcgatttattcaacaaagccgccgt
cccgtcaagtcagcgtaatgctctgccagtgttacaaccaattaaccaattctgattagaaaaactcatcgagcatcaaatgaaactgcaattt
attcatatcaggattatcaataccatatttttgaaaaagccgtttctgtaatgaaggagaaaactcaccgaggcagttccataggatggcaaga
tcctggtatcggtctgcgattccgactcgtccaacatcaatacaacctattaatttcccctcgtcaaaaataaggttatcaagtgagaaatcacc
atgagtgacgactgaatccggtgagaatggcaaaagcttatgcatttctttccagacttgttcaacaggccagccattacgctcgtcatcaaa
atcactcgcatcaaccaaaccgttattcattcgtgattgcgcctgagcgagacgaaatacgcgatcgctgttaaaaggacaattacaaacag
gaatcgaatgcaaccggcgcaggaacactgccagcgcatcaacaatattttcacctgaatcaggatattcttctaatacctggaatgctgtttt
cccggggatcgcagtggtgagtaaccatgcatcatcaggagtacggataaaatgcttgatggtcggaagaggcataaattccgtcagcca
gtttagtctgaccatctcatctgtaacatcattggcaacgctacctttgccatgtttcagaaacaactctggcgcatcgggcttcccatacaatc
gatagattgtcgcacctgattgcccgacattatcgcgagcccatttatacccatataaatcagcatccatgttggaatttaatcgcggcctgga
gcaagacgtttcccgttgaatatggctcataacaccccttgtattactgtttatgtaagcagacagttttattgttcatgatgatatatttttatcttgt
gcaatgtaacatcagagattttgagacacaacgtggctttccccccccccccattattgaagcatttatcagggttattgtctcatgagcggata
catatttgaatgtatttagaaaaataaacaaataggggttccgcgcacatttccccgaaaagtgccacctgacgtctaagaaaccattattatc
atgacattaacctataaaaataggcgtatcacgaggccctttcgtctcgcgcgtttcggtgatgacggtgaaaacctctgacacatgcagctc
ccggagacggtcacagcttgtctgtaagcggatgccgggagcagacaagcccgtcagggcgcgtcagcgggtgttggcgggtgtcgg
ggctggcttaactatgcggcatcagagcagattgtactgagagtgcaccatatgcggtgtgaaataccgcacagatgcgtaaggagaaaat
accgcatcagattggctat
SEQ ID NO: 16 = Amino acid sequence of the construct encoded by pMIP3α-SARS-
CoV-2-ΔTSL. The underlined portion shows the amino acid sequence of a modified SARS-
CoV-2 Spike protein.
MCCTKSLLLAALMSVLLLHLCGESEASNFDCCLGYTDRILHPKFIVGFTRQLANEGCDI
NAIIFHTKKKLSVCANPKQTWVKYIVRLLSKKVKNMEFNDAQAPKSLEVNLTTRTQLP
PAYTNSFTRGVYYPDKVFRSSVLHSTQDLFLPFFSNVTWFHAIHVSGTNGTKRFDN
PVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTV
EKGIYQTSNERVQPTESIVRFPNITNLCPFGEVENATRFASVYAWNRKRISNCVADY
SVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYSTGSNVFQT
RAGCLIGAEHVNNSYECDIPIGAGICASYQTQTNSPSGGGGSGGGGSGGGGSSVAS
QSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQI
LPDPSKSGGGGSGGGGSGGGGSSFIEDLLENKVTLADAGFIKQYGDCLGDIAARDL
ICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEI
RASANLAATKMSECVLGQSKRVDFCGKGYHLMSFPQSAPHGVVFLHVTYVPAQE
KNFTTAPAICHDGKAHFPREGVFVSNGTHWFVTQRNFYEPQIITTDNTFVSGNCDV
VIGIVNNTVYDPLQPELDSFKEELDKYFKNHTSPDVDLGDISGINASVVNIQKEIDRL
NEVAKNLNESLIDLQELGKYEQGGRYPYDVPDYA
SEQ ID NO: 17 = human MIP3α Amino Acid Sequence
MCCTKSLLLA ALMSVLLLHL CGESEAASNF DCCLGYTDRI LHPKFIVGET
RQLANEGCDI NAIIFHTKKK LSVCANPKQT WVKYIVRLLS KKVKNM
SEQ ID NO: 18 = Deleted N-terminal sequence from naturally-occuring SEQ ID
NO: 44 and functional fragment SEQ ID NO: 45.
MFVFLVLLPL VSSQC
SEQ ID NO: 19 = DSEPVLKGVKLHYT
SEQ ID NO: 20 = S1/S2 site of SARS-CoV2 spike protein: RRAR
SEQ ID NO: 21 = S2′ site of SARS-CoV2 spike protein: PSKR
SEQ ID NO: 22 = GGGGS
SEQ ID NO: 23 = GGGGSGGGGSGGGGS
SEQ ID NO: 24 = SGGGGSGGGGSGGGGS
SEQ ID NO: 25 = GGGS
SEQ ID NO: 26 = GGGSGGGSGGGS
SEQ ID NO: 27 = SGGGSGGGSGGGS
SEQ ID NO: 28 = Human MIP3α Signal DNA sequence:
atgtgctgtaccaagagtttgctcctggctgctttgatgtcagtgctgctactccacctctgcggcgaatcagaagcaagc
SEQ ID NO: 29 = Human MIP3α Signal amino acid sequence:
MCCTKSLLLAALMSVLLLHLCGESEAS
SEQ ID NO: 30 = HA tag DNA sequence: tacccatacgacgtaccagattacgct
SEQ ID NO: 31 = HA tag amino acid sequence: YPYDVPDYA
SEQ ID NO: 32 = Spacer DNA sequence: aacgacgctcaggcgccgaagagt
SEQ ID NO: 33 = Spacer amino acid sequence: NDAQAPKS
SEQ ID NO: 34 = DNA sequence of S1/S2 cleavage site of SARS-CoV-2 spike protein:
CGGCGGGCACGT
SEQ ID NO: 35 = DNA sequence of S2′ cleavage site of SARS-CoV-2 spike protein:
CCAAGCAAGAGG
SEQ ID NO: 36 = DNA sequence of flexible linker replacing S1/S2 cleavage site in
pSARS-CoV-2-ΔTSL and pMIP3α-SARS-CoV-2-ΔTSL constructs (SEQ ID NO: 36):
GGTGGGGGAGGCTCAGGCGGAGGTGGTAGCGGGGGCGGTGGTTCA
SEQ ID NO: 37 = DNA sequence of flexible linker replacing S2′ cleavage site in
pSARS-CoV-2-ΔTSL and pMIP3α-SARS-CoV-2-ΔTSL constructs:
GGGGGTGGGGGAAGTGGGGGAGGGGGGAGCGGCGGAGGAGGTAGT
SEQ ID NO: 38 = Amino acid sequence of flexible linker replacing S1/S2 cleavage site
in pSARS-CoV-2-ΔTSL and pMIP3α-SARS-CoV-2-ΔTSL constructs:
GGGGSGGGGSGGGGS
SEQ ID NO: 39 = Amino acid sequence of flexible linker replacing S2′ cleavage site in
pSARS-CoV-2-ΔTSL and pMIP3α-SARS-CoV-2-ΔTSL constructs: GGGGSGGGGSGGGGS
SEQ ID NO: 40 = Spike protein transmembrane and cytoplasmic domains
YIKWPWYIWL GFIAGLIAIV MVTIMLCCMT SCCSCLKGCC SCGSCCKEDE
DDSEPVLKGV KLHYT
SEQ ID NO: 41 = amino acid sequence of transmembrane domain of SARS-CoV-2
Spike protein.
YIKWPWYIWL GFIAGLIAIV MVTIMLCCMT SCCSCLKGCC SCGSCC
SEQ ID NO: 42 = amino acid sequence of cytoplasmic domain of SARS-CoV-2 Spike
protein.
KFDEDDSEPV LKGVKLHYT
SEQ ID NO: 43
NFDCCLGYTDRILHPKFIVGFTRQLANEGCDINAIIFHTKKKLSVCANPKQTWVKYIVRL
LSKKVKNMEFNDAQAPKSLE
SEQ ID NO: 44 (SARS-CoV-2 spike protein of NCBI Reference Sequence:
YP_009724390.1)
MFVFLVLLPL VSSQCVNLTT RTQLPPAYTN SFTRGVYYPD KVFRSSVLHS
TQDLFLPFFS NVTWFHAIHV SGTNGTKRED NPVLPENDGV YFASTEKSNI
IRGWIFGTTL DSKTQSLLIV NNATNVVIKV CEFQFCNDPF LGVYYHKNNK
SWMESEFRVY SSANNCTFEY VSQPFLMDLE GKQGNEKNLR EFVFKNIDGY
FKIYSKHTPI NLVRDLPQGF SALEPLVDLP IGINITRFQT LLALHRSYLT
PGDSSSGWTA GAAAYYVGYL QPRTELLKYN ENGTITDAVD CALDPLSETK
CTLKSFTVEK GIYQTSNERV QPTESIVRFP NITNLCPFGE VENATRFASV
YAWNRKRISN CVADYSVLYN SASFSTFKCY GVSPTKLNDL CFTNVYADSF
VIRGDEVRQI APGQTGKIAD YNYKLPDDFT GCVIAWNSNN LDSKV9*GGNYN
YLYRLFRKSN LKPFERDIST EIYQAGSTPC NGVEGENCYF PLQSYGFQPT
NGVGYQPYRV VVLSFELLHA PATVCGPKKS TNLVKNKCVN FNFNGLTGTG
VLTESNKKEL PFQQFGRDIA DTTDAVRDPQ TLEILDITPC SFGGVSVITP
GTNTSNQVAV LYQDVNCTEV PVAIHADQLT PTWRVYSTGS NVFQTRAGCL
IGAEHVNNSY ECDIPIGAGI CASYQTQTNS PRRARSVASQ SIIAYTMSLG
AENSVAYSNN SIAIPTNFTI SVTTEILPVS MTKTSVDCTM YICGDSTECS
NLLLQYGSFC TQLNRALTGI AVEQDKNTQE VFAQVKQIYK TPPIKDFGGF
NFSQILPDPS KPSKRSFIED LLENKVTLAD AGFIKQYGDC LGDIAARDLI
CAQKFNGLTV LPPLLTDEMI AQYTSALLAG TITSGWTFGA GAALQIPFAM
QMAYRENGIG VTQNVLYENQ KLIANQFNSA IGKIQDSLSS TASALGKLQD
VVNQNAQALN TLVKQLSSNF GAISSVLNDI LSRLDKVEAE VQIDRLITGR
LQSLQTYVTQ QLIRAAEIRA SANLAATKMS ECVLGQSKRV DFCGKGYHLM
SFPQSAPHGV VFLHVTYVPA QEKNFTTAPA ICHDGKAHFP REGVFVSNGT
HWFVTQRNFY EPQIITTDNT FVSGNCDVVI GIVNNTVYDP LQPELDSFKE
ELDKYFKNHT SPDVDLGDIS GINASVVNIQ KEIDRLNEVA KNLNESLIDL
QELGKYEQYI KWPWYIWLGF IAGLIAIVMV TIMLCCMTSC CSCLKGCCSC
GSCCKFDEDD SEPVLKGVKL HYT
SEQ ID NO: 45-Functional Fragment of SARS-CoV-2 spike protein of SEQ ID
NO: 44
VNLTT RTQLPPAYTN SFTRGVYYPD KVFRSSVLHS TQDLFLPFFS NVTWFHAIHV
SGTNGTKRED NPVLPENDGV YFASTEKSNI IRGWIFGTTL DSKTQSLLIV
NNATNVVIKV CEFQFCNDPF LGVYYHKNNK SWMESEFRVY SSANNCTFEY
VSQPFLMDLE GKQGNEKNLR EFVEKNIDGY FKIYSKHTPI NLVRDLPQGF
SALEPLVDLP IGINITRFQT LLALHRSYLT PGDSSSGWTA GAAAYYVGYL
QPRTFLLKYN ENGTITDAVD CALDPLSETK CTLKSFTVEK GIYQTSNERV
QPTESIVRFP NITNLCPFGE VFNATRFASV YAWNRKRISN CVADYSVLYN
SASFSTFKCY GVSPTKLNDL CFTNVYADSF VIRGDEVRQI APGQTGKIAD
YNYKLPDDFT GCVIAWNSNN LDSKVGGNYN YLYRLFRKSN LKPFERDIST
EIYQAGSTPC NGVEGENCYF PLQSYGFQPT NGVGYQPYRV VVLSFELLHA
PATVCGPKKS TNLVKNKCVN FNFNGLTGTG VLTESNKKEL PFQQFGRDIA
DTTDAVRDPQ TLEILDITPC SFGGVSVITP GTNTSNQVAV LYQDVNCTEV
PVAIHADQLT PTWRVYSTGS NVFQTRAGCL IGAEHVNNSY ECDIPIGAGI
CASYQTQTNS PRRARSVASQ SIIAYTMSLG AENSVAYSNN SIAIPTNFTI
SVTTEILPVS MTKTSVDCTM YICGDSTECS NLLLQYGSFC TQLNRALTGI
AVEQDKNTQE VFAQVKQIYK TPPIKDEGGF NESQILPDPS KPSKRSFIED
LLFNKVTLAD AGFIKQYGDC LGDIAARDLI CAQKENGLTV LPPLLTDEMI
AQYTSALLAG TITSGWTFGA GAALQIPFAM QMAYRENGIG VTQNVLYENQ
KLIANQFNSA IGKIQDSLSS TASALGKLQD VVNQNAQALN TLVKQLSSNF
GAISSVLNDI LSRLDKVEAE VQIDRLITGR LQSLQTYVTQ QLIRAAEIRA
SANLAATKMS ECVLGQSKRV DFCGKGYHLM SFPQSAPHGV VFLHVTYVPA
QEKNFTTAPA ICHDGKAHFP REGVFVSNGT HWFVTQRNFY EPQIITTDNT
FVSGNCDVVI GIVNNTVYDP LQPELDSFKE ELDKYFKNHT SPDVDLGDIS
GINASVVNIQ KEIDRLNEVA KNLNESLIDL QELGKYEQYI KWPWYIWLGF
IAGLIAIVMV TIMLCCMTSC CSCLKGCCSC GSCCKEDEDD SEPVLKGVKL
HYT
SEQ ID NO: 46 = Spike protein before S1/S2 cleavage site.
          VNLTT RTQLPPAYTN SFTRGVYYPD KVERSSVLHS TQDLFLPFFS
NVTWFHAIHV SGTNGTKRED NPVLPENDGV YFASTEKSNI IRGWIEGTTL
DSKTQSLLIV NNATNVVIKV CEFQFCNDPF LGVYYHKNNK SWMESEFRVY
SSANNCTFEY VSQPFLMDLE GKQGNFKNLR EFVFKNIDGY FKIYSKHTPI
NLVRDLPQGF SALEPLVDLP IGINITRFQT LLALHRSYLT PGDSSSGWTA
GAAAYYVGYL QPRTELLKYN ENGTITDAVD CALDPLSETK CTLKSFTVEK
GIYQTSNFRV QPTESIVRFP NITNLCPFGE VENATRFASV YAWNRKRISN
CVADYSVLYN SASFSTFKCY GVSPTKLNDL CFTNVYADSF VIRGDEVRQI
APGQTGKIAD YNYKLPDDFT GCVIAWNSNN LDSKVGGNYN YLYRLFRKSN
LKPFERDIST EIYQAGSTPC NGVEGENCYF PLQSYGFQPT NGVGYQPYRV
VVLSFELLHA PATVCGPKKS TNLVKNKCVN FNENGLTGTG VLTESNKKEL
PFQQFGRDIA DTTDAVRDPQ TLEILDITPC SFGGVSVITP GTNTSNQVAV
LYQDVNCTEV PVAIHADQLT PTWRVYSTGS NVFQTRAGCL IGAEHVNNSY
ECDIPIGAGI CASYQTQTNS P
SEQ ID NO: 47 = Spike protein after S1/S2 cleavage site and before S2′ cleavage site
          SVASQ SIIAYTMSLG AENSVAYSNN SIAIPTNFTI SVTTEILPVS
MTKTSVDCTM YICGDSTECS NLLLQYGSFC TQLNRALTGI AVEQDKNTQE
VFAQVKQIYK TPPIKDFGGF NFSQILPDPS K
SEQ ID NO: 48 = Spike protein after S2′ cleavage site and before transmembrane
domain
          SFIED LLENKVTLAD AGFIKQYGDC LGDIAARDLI CAQKENGLTV
LPPLLTDEMI AQYTSALLAG TITSGWTFGA GAALQIPFAM QMAYRENGIG
VTQNVLYENQ KLIANQFNSA IGKIQDSLSS TASALGKLQD VVNQNAQALN
TLVKQLSSNF GAISSVLNDI LSRLDKVEAE VQIDRLITGR LQSLQTYVTQ
QLIRAAEIRA SANLAATKMS ECVLGQSKRV DECGKGYHLM SFPQSAPHGV
VFLHVTYVPA QEKNFTTAPA ICHDGKAHFP REGVFVSNGT HWFVTQRNFY
EPQIITTDNT FVSGNCDVVI GIVNNTVYDP LQPELDSFKE ELDKYFKNHT
SPDVDLGDIS GINASVVNIQ KEIDRLNEVA KNLNESLIDL QELGKYEQ
```

### REFERENCES

1. Biragyn et al, Science. 2002; 298(5595):1025-9. PubMed PMID: 12411706.
2. Biragyn et al, Nat Biotechnol. 1999; 17(3):253-8. PubMed PMID: 10096292.
3. Qin et al, Blood. 2009; 114(19):4142-9. PubMed PMID: 19749091.
4. Biragyn et al, J Immunol. 2001; 167(11):6644-53. PubMed PMID: 11714836.
5. Thomas et al, BMC Cancer. 2018; 18(1):187. PubMed PMID: 29439670; PubMed Central PMCID: PMC5812202.
6. Kwak et al, N Engl J Med. 1992; 327(17):1209-15. PubMed PMID: 1406793.
7. Bendandi et al, Nat Med. 1999; 5(10):1171-7. PubMed PMID: 10502821.
8. Neelapu et al, Nat Med. 2005; 11(9):986-91. PubMed PMID: 16116429.
9. Schuster et al, J Clin Oncol. 2009; 27(18). PubMed PMID: WOS:000276607000008.
10. Baskar et al, J Clin Invest. 2004; 113(10):1498-510. PubMed PMID: 15146248; PubMed Central PMCID: PMC406527.
11. Neelapu et al, Clin Cancer Res. 2004; 10(24):8309-17. PubMed PMID: 15623607.
12. Neelapu et al, Blood. 2007; 109(12):5160-3. PubMed PMID: 17339422; PubMed Central PMCID: PMC1941785.
13. Neelapu et al, Bone Marrow Transplant. 2005; 36(4):315-23. PubMed PMID: 15968284.
14. Prado et al, J Biol Chem. 1996; 271(32):19186-90. PubMed PMID: 8702597.
15. Signoret et al, J Cell Biol. 1997; 139(3):651-64. PubMed PMID: 9348282; PubMed Central PMCID: PMC2141706.
16. Solari et al, J Biol Chem. 1997; 272(15):9617-20. PubMed PMID: 9092487.
17. Biragyn et al, Blood. 2002; 100(4):1153-9. PubMed PMID: 12149191.
18. Qin et al, J Biomed Biotechnol. 2010; 2010:860160. PubMed PMID: 20454526; PubMed Central PMCID: PMC2864514.
19. Zhu et al, Vaccine. 2007; 25(46):7955-61. PubMed PMID: 17933439.
20. Park et al, Vaccine. 2011; 29(18):3476-82. PubMed PMID: 21382485.
21. Nicholls et al, J Immunol Methods. 1993; 165(1):81-91. PubMed PMID: 8409471.
22. Ruffini et al, J Leukoc Biol. 2004; 76(1):77-85. PubMed PMID: 15075363.
23. Wu et al, Cell Host Microbe. 2020; 27(3):325-8. PubMed PMID: 32035028; PubMed Central PMCID: PMCPMC7154514.
24. Yang et al, Nature. 2004; 428(6982):561-4. PubMed PMID: 15024391; PubMed Central PMCID: PMC7095382.
25. Kliger et al, BMC Microbiol. 2003; 3:20. PubMed PMID: 14499001; PubMed Central PMCID: PMCPMC222911.
26. Phan, Infect. Genet Evol. 2020; 81:104260.
27. Los Alamos National Laboratory. SARS-CoV-2 Sequence Analysis pipeline 2020. Available from: https://cov.lanl.gov/content/index.
28. Buchner et al, Anal Biochem. 1992; 205(2):263-70. PubMed PMID: 1332541.
29. Wrapp et al, Science. 2020; 367(6483):1260-3. PubMed PMID: 32075877; PubMed Central PMCID: PMC7164637.
30. Hoffmann et al, Cell. 2020; 181(2):271-80 e8. PubMed PMID: 32142651; PubMed Central PMCID: PMCPMC7102627.
31. Koyama et al, Pathogens. 2020; 9(5). PubMed PMID: 32357545.
32. Qin et al, Sci Transl Med. 2019; 11(511). PubMed PMID: 31554741; PubMed Central PMCID: PMC7015136.
33. Qin et al, Vaccine. 2010 November; 28(50):7970-7978. DOI: 10.1016/j.vaccine.2010.09.084
34. Lee et al, J Immunother. 2000; 23(3):379-86.
35. Chapman et al, Nucleic Acids Res. 1991; 19(14):3979-86.
36. Buchman et al, Mol Cell Biol. 1988; 8(10):4395-405.
37. Gil et al, Nature. 1984; 312(5993):473-4.
38. Yanisch-Perron et al, Gene. 1985; 33(1):103-19.
39. Nakano et al, J Bacteriol. 1984; 157(1):79-83.

What is claimed is:

1. A protein having at least 85% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

2. The protein of claim 1 having at least 90% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

3. The protein of claim 1 having at least 95% sequence identity to SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

4. The protein of claim 1 having SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

5. The protein of claim 1, wherein the protein does not comprise an HA-tag having SEQ ID NO:31.

6. A nucleic acid encoding the protein of claim 1.

7. A nucleic acid having at least 85% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

8. The nucleic acid of claim 6, having at least 90% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

9. The nucleic acid of claim 6, having at least 95% sequence identity to SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

10. The nucleic acid of claim 7, wherein the nucleic acid does not comprise an HA-tag having SEQ ID NO:30.

11. A protein comprising an amino acid sequence of formula (I):
R¹-L¹-R²-L²-R³-R⁴-R⁵,
wherein:
R¹ is an amino acid sequence having at least 85% sequence identity to SEQ ID NO:46;
L¹ is SEQ ID NO:20, absent, —S(G₄S)ₓ—, -(G₄S)ₓ—, —S(G₃S)ₓ—, or -(G₃S)ₓ—; wherein x is an integer from 1 to 10;
R² is an amino acid sequence having at least 85% sequence identity to SEQ ID NO:47;
L² is SEQ ID NO:21, absent, —S(G₄S)ₓ—, -(G₄S)ₓ—, —S(G₃S)ₓ—, or -(G₃S)ₓ—; wherein x is an integer from 1 to 10;
R³ is an amino acid sequence having at least 85% sequence identity to SEQ ID NO:48;
R⁴ is absent, an amino acid sequence having at least 85% sequence identity to SEQ ID NO:41, or a truncated transmembrane domain; and
R³ is absent or a truncated cytoplasmic domain; provided that R⁵ is absent when R⁴ is absent or a truncated transmembrane domain.

12. The protein of claim 11, wherein R¹ is an amino acid sequence having at least 90% sequence identity to SEQ ID NO:46; R² is an amino acid sequence having at least 90% sequence identity to SEQ ID NO:47; R³ is an amino acid sequence having at least 90% sequence identity to SEQ ID NO:48.

13. The protein of claim 12, wherein R¹ is an amino acid sequence having at least 95% sequence identity to SEQ ID NO:46; R² is an amino acid sequence having at least 95% sequence identity to SEQ ID NO:47; and R³ is an amino acid sequence having at least 95% sequence identity to SEQ ID NO:48.

14. The protein of claim 13, wherein R¹ is an amino acid sequence having SEQ ID NO:46; R² is an amino acid sequence having SEQ ID NO:47; and R³ is an amino acid sequence having SEQ ID NO:48.

15. The protein of claim 11, wherein R⁴ is an amino acid sequence having at least 90% sequence identity to SEQ ID NO:41.

16. The protein of claim 15, wherein R⁴ is an amino acid sequence having at least 95% sequence identity to SEQ ID NO:41.

17. The protein of claim 16, wherein R⁴ is an amino acid sequence having SEQ ID NO:41.

18. The protein of claim 11, wherein R⁵ is absent.

19. The protein of claim 11, wherein R³ is a truncated cytoplasmic domain.

20. The protein of claim 11, wherein R⁴ is absent and R³ is absent.

21. The protein of claim 11, wherein R⁴ is a truncated transmembrane domain and R⁵ is absent.

22. The protein of claim 11, wherein L is absent, —S(G₄S)ₓ—, -(G₄S)ₓ—, —S(G₃S)ₓ—, or -(G₃S)ₓ—; wherein x is an integer from 1 to 10; and L² is absent, —S(G₄S)ₓ—, -(G₄S)ₓ—, —S(G₃S)ₓ—, or -(G₃S)ₓ—; wherein x is an integer from 1 to 10.

23. The protein of claim 22, wherein L¹ is absent and L² is absent.

24. The protein of claim 22, wherein L¹ is —S(G₄S)ₓ—, -(G₄S)ₓ—, —S(G₃S)ₓ—, or -(G₃S)ₓ—; wherein x is an integer from 1 to 6; and L² is —S(G₄S)ₓ—, -(G₄S)ₓ—, —S(G₃S)ₓ—, or -(G₃S)ₓ—; wherein x is an integer from 1 to 6.

25. The protein of claim 24, wherein L¹ is -(G₄S)ₓ— or -(G₃S)ₓ—; where x is an integer from 1 to 3; and L² is -(G₄S)ₓ— or -(G₃S)ₓ—; where x is an integer from 1 to 3.

26. The protein of claim 11, further comprising an amino acid sequence of a human MIP3α protein.

27. The protein of claim 26, wherein the human MIP3α protein has the amino acid sequence of SEQ ID NO:29.

28. The protein of claim 26, wherein the human MIP3α protein is located at position on the N-terminus of R¹.

29. A nucleic acid encoding the protein of claim 11.

30. A SARS-CoV-2 spike (S) protein comprising a truncated cytoplasmic domain.

31. A SARS-CoV-2 spike (S) protein comprising a truncated transmembrane domain; wherein the protein does not comprise the amino acid sequence of SEQ ID NO:42.

32. The SARS-CoV-2 spike (S) protein of claim 30, wherein the SARS-CoV-2 spike (S) protein has at least 85% sequence identity to SEQ ID NO:44 or has at least 85% sequence identity to SEQ ID NO:45.

33. A SARS-CoV-2 spike (S) protein having at least 85% sequence identity to SEQ ID NO:44 or has at least 85% sequence identity to SEQ ID NO:45; provided that SEQ ID NO:44 and SEQ ID NO:45 do not comprise the amino acid sequence of SEQ ID NO:40.

34. The SARS-CoV-2 spike (S) protein of claim 30, wherein the S1/S2 protease cleavage site in the SARS-CoV-2 spike (S) protein is absent or replaced with a peptide linker.

35. The SARS-CoV-2 spike (S) protein of claim 30, wherein the S2′ protease cleavage site in the SARS-CoV-2 spike (S) protein is absent or replaced with a peptide linker.

36. The SARS-CoV-2 spike (S) protein of claim 35, wherein the S1/S2 protease cleavage site is replaced with a peptide linker and the S2′ protease cleavage site is replaced with a peptide linker.

37. The SARS-CoV-2 spike (S) protein of claim 36, wherein the peptide linker is —S(G₄S)ₓ—, -(G₄S)ₓ—, —S(G₃S)ₓ—, or -(G₃S)ₓ—; wherein x is an integer from 1 to 6.

38. The SARS-CoV-2 spike (S) protein of claim 37, where the peptide linker is -(G₄S)₃—.

39. The SARS-CoV-2 spike (S) protein of claim 34, wherein the S1/S2 protease cleavage site in the SARS-CoV-2 spike (S) protein is absent and the S2′ protease cleavage site in the SARS-CoV-2 spike (S) protein is absent.

40. The SARS-CoV-2 spike (S) protein of claim 30, further comprising a human MIP3α protein.

41. A nucleic acid encoding the SARS-CoV-2 spike (S) protein of any one of claims 30 to 41.

42. A plasmid comprising the nucleic acid of claim 6.

43. The plasmid of claim 42, further comprising a nucleic acid encoding a human MIP3α protein.

44. The plasmid of claim 42, further comprising a ubiquitous promoter.

45. The plasmid of claim 44, wherein the ubiquitous promoter is a CMV promoter.

46. The plasmid of claim 42, further comprising a polyadenylation signal located downstream of the nucleic acid encoding the protein.

47. The plasmid of claim 46, wherein the polyadenylation signal is a rabbit β-globulin polyadenylation signal.

48. A pharmaceutical composition comprising: (i) the protein of any one of claims 1-5, 11-28, and 30-40, and a pharmaceutically acceptable excipient, (ii) the nucleic acid of one of claims 6-10, 29, and 41, and a pharmaceutically acceptable excipient; or (iii) the plasmid of any one of claims 42-47.

49. A vaccine comprising: (i) the nucleic acid of one of claims 6-10, 29, and 41 and an adjuvant, or (ii) the plasmid of any one of claims 42-47.

50. A method of increasing immunity to a SARS coronavirus in a human in need thereof, the method comprising administering to the human an effective amount of: (i) the protein of any one of claims 1-5, 11-28, and 30-40; (ii) the nucleic acid of one of claims 6-10, 29, and 41; (iii) the plasmid of any one of claims 42-47; (iv) the pharmaceutical composition of claim 48; or (v) the vaccine of claim 49.

51. A method of providing acquired immunity to a SARS coronavirus in a human in need thereof, the method comprising administering to the human an effective amount of: (i) the protein of any one of claims 1-5, 11-28, and 30-40; (ii) the nucleic acid of one of claims 6-10, 29, and 41; (iii) the plasmid of any one of claims 42-47; (iv) the pharmaceutical composition of claim 48; or (v) the vaccine of claim 49.

52. The method of claim 50 or 51, wherein the SARS coronavirus is SARS-CoV-2.

53. A method of preventing COVID-19 in a human in need thereof, the method comprising administering to the human an effective amount of: (i) the protein of any one of claims 1-5, 11-28, and 30-40; (ii) the nucleic acid of one of claims 6-10, 29, and 41; (iii) the plasmid of any one of claims 42-47; (iv) the pharmaceutical composition of claim 48; or (v) the vaccine of claim 49.

54. The method of any one of claims 50 to 53, comprising administering the effective amount of the nucleic acid via intramuscular injection, subcutaneous injection, or pulmonary administration.

55. The method of any one of claims 50 to 54, comprising administering the effective amount of the nucleic acid via a needle-free injector.

56. An injector comprising the vaccine of claim 49.

57. The injector of claim 56, wherein the injector is a needle-free injector.

58. A syringe comprising the vaccine of claim 49.

59. A vial comprising the vaccine of claim 49.

60. A kit comprising:
(a) the vaccine of claim 49, a needle-free injector, and instructions for use;
(b) the vaccine of claim 49, a syringe, and instructions for use;
(c) the injector of claim 56 or 57 and instructions for use;
(d) the syringe of claim 58 and instructions for use; or
(e) the vial of claim 59 and instructions for use.


SEQUENCE LISTING (48 sequences)

SEQ ID NO: 1 - DNA, 7793 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcgtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccccgacgcgcccgctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaaccctccaaacgatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagtatataaaatggccatggtacatttggttgggattcatcgcaggattgatcgccattgttatggttacaatcatgctctgttgtatgaccagttgctgtagctgcttgaagggctgctgctcatgcggttcttgttgcggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 2 - protein, 1278 aa, Artificial Sequence
  Synthetic Polypeptide
MCCTKSLLLAALMSVLLLHLCGESEASVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCGGRYPYDVPDYA

SEQ ID NO: 3 - DNA, 8033 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtctcgaggtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccccgacgcgcccgctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaaccctccaaacgatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagtatataaaatggccatggtacatttggttgggattcatcgcaggattgatcgccattgttatggttacaatcatgctctgttgtatgaccagttgctgtagctgcttgaagggctgctgctcatgcggttcttgttgcggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 4 - protein, 1358 aa, Artificial Sequence
  Synthetic Polypeptide
MCCTKSLLLAALMSVLLLHLCGESEASNFDCCLGYTDRILHPKFIVGFTRQLANEGCDINAIIFHTKKKLSVCANPKQTWVKYIVRLLSKKVKNMEFNDAQAPKSLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCGGRYPYDVPDYA

SEQ ID NO: 5 - DNA, 7655 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcgtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccccgacgcgcccgctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaaccctccaaacgatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 6 - protein, 1232 aa, Artificial Sequence
  Synthetic Polypeptide
MCCTKSLLLAALMSVLLLHLCGESEASVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 7 - DNA, 7895 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtctcgaggtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccccgacgcgcccgctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaaccctccaaacgatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 8 - protein, 1312 aa, Artificial Sequence
  Synthetic Polypeptide
MCCTKSLLLAALMSVLLLHLCGESEASNFDCCLGYTDRILHPKFIVGFTRQLANEGCDINAIIFHTKKKLSVCANPKQTWVKYIVRLLSKKVKNMEFNDAQAPKSLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 9 - DNA, 7631 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcgtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 10 - protein, 1224 aa, Artificial Sequence
  Synthetic Polypeptide
MCCTKSLLLAALMSVLLLHLCGESEASVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 11 - DNA, 7871 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtctcgaggtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccctctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaatccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 12 - protein, 1304 aa, Artificial Sequence
  Synthetic Polypeptide
MCCTKSLLLAALMSVLLLHLCGESEASNFDCCLGYTDRILHPKFIVGFTRQLANEGCDINAIIFHTKKKLSVCANPKQTWVKYIVRLLSKKVKNMEFNDAQAPKSLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 13 - DNA, 7727 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcgtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccctctggtgggggaggctcaggcggaggtggtagcgggggcggtggttcatctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaatctgggggtgggggaagtgggggaggggggagcggcggaggaggtagttccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 14 - protein, 1256 aa, Artificial Sequence
  Synthetic Polypeptide
MCCTKSLLLAALMSVLLLHLCGESEASVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGGGGSGGGGSGGGGSSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSGGGGSGGGGSGGGGSSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 15 - DNA, 7967 bp, Artificial Sequence
  Synthetic Polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcaggccgccaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtctcgaggtgaatttgaccaccaggacccagcttccgcctgcctataccaattcatttaccagaggtgtatattacccggataaagtgttccgatcctcagttctccattctacacaagacttgttcctccccttcttctcaaatgtgacttggtttcacgcaatccatgtgtctggaacgaatggcaccaagaggtttgacaatccagtcttgccttttaatgatggtgtctatttcgctagcacggaaaaaagcaatataatacggggttggatcttcggcactacacttgacagtaaaacgcagagccttcttatagtgaataacgctaccaacgtggtaataaaagtgtgtgagttccaattctgcaatgatccattcttgggcgtgtattaccataaaaataacaaaagttggatggaaagcgaattccgcgtgtacagctctgccaacaactgcacttttgagtatgtgagccagccatttttgatggatctggaaggaaagcaaggtaactttaaaaacttgcgggagttcgtttttaagaacatcgatgggtatttcaaaatctactcaaaacacactcctattaatttggttcgcgatttgccacagggtttctctgccctcgaacctcttgttgatctccctattggaataaatatcacgagattccagacattgctcgccctgcatcgctcatatctgacacctggtgatagcagcagcggatggacggcaggcgcagccgcgtattacgttggctacttgcaacccagaacatttctccttaagtataatgagaatggtacgatcactgatgcggtcgactgcgcgttggaccctctgagcgagacaaaatgcactctcaagagtttcactgtcgagaaaggcatttaccaaacatctaatttccgagtgcaaccaactgagtccattgttcgattcccaaacattactaacttgtgtcctttcggcgaagtgttcaacgcaacgagatttgcaagtgtatacgcgtggaaccgaaagagaatcagcaactgtgtagccgattattctgttctgtacaatagcgctagcttcagcactttcaaatgctacggtgtaagtcctacaaagctgaatgacctctgtttcacaaatgtgtacgcggatagcttcgtcattaggggagatgaagtacggcaaatcgcccccggtcaaacagggaagatagctgactacaactataaacttccggatgattttacggggtgtgttatagcgtggaattctaacaacctcgattcaaaggtcgggggaaattacaattacctttaccgcctgttccgaaagtcaaatcttaagccattcgaaagggacatatcaacggaaatttatcaagccgggtctactccatgcaatggcgtcgagggttttaattgctattttccactgcaatcttacggtttccaaccaacgaacggagtaggatatcaaccttaccgggttgtagtgttgtctttcgaactgctccacgcgccagctaccgtgtgcggtccgaaaaaaagcacaaatctggttaagaacaagtgtgttaacttcaactttaacggtctcactggaacaggtgtacttacagaatccaataagaaatttcttccgttccaacagttcggtagagacattgcggacaccactgatgctgtgcgagatcctcagacactcgaaatactggacattacaccttgcagcttcggcggtgttagtgtaatcacgccgggcactaatacatccaatcaggtcgctgtgttgtaccaagatgttaactgtacggaggtacctgttgcaatacatgcggaccaactcactcctacttggagggtatattctaccggatccaacgtgttccagactcgggcgggctgcctgataggtgccgagcatgtcaataattcttacgaatgcgatattccaataggggccggcatatgtgcatcttatcaaacccaaaccaactccccctctggtgggggaggctcaggcggaggtggtagcgggggcggtggttcatctgttgcgtcacagagtattatagcctatacgatgtctttgggggcggaaaactccgtcgcttactccaacaacagtatagcaatccctacgaattttaccatctccgtaacaactgaaatccttcctgtgtcaatgaccaaaacttctgtcgattgcacgatgtatatctgcggagacagcacggaatgtagtaacttgctcctccagtacggctctttctgcacacagctcaatcgggcgctgaccgggatagcggtggaacaagacaagaacacccaggaagtcttcgcgcaggtcaagcaaatctacaagacccctcccattaaagattttggtggttttaactttagccaaatactgccagatccgtccaaatctgggggtgggggaagtgggggaggggggagcggcggaggaggtagttccttcatcgaggacctcctttttaacaaagttaccctcgccgacgccgggttcataaaacagtacggcgactgcttgggtgacatcgcagccagagacttgatttgtgcgcaaaaattcaacggccttacggtcctgcctccattgctgaccgatgaaatgattgcccagtatacctctgctttgttggcagggacaataacttctgggtggacgtttggagcaggcgctgccctgcagatcccttttgccatgcagatggcataccggtttaacgggatcggtgttactcagaacgtactctatgagaatcaaaagctgatcgccaaccaatttaatagcgccatcggtaaaatccaggatagtttgagcagcaccgcttctgcacttgggaagcttcaggatgttgtgaatcagaacgcccaagcattgaatactctcgtcaaacaattgagtagtaatttcggagccatctcatctgtgttgaatgacatactcagtcgcctcgataaggtagaggctgaggtgcagatagaccggcttataactggtagattgcagagccttcagacttacgtgacgcaacagcttatccgagcagcagaaatcagggcatcagcaaatcttgccgccaccaaaatgtcagagtgcgtactgggccaaagtaaaagagtggatttttgtggaaaaggataccacttgatgagctttccccagtctgccccgcacggtgtggtcttcctgcacgtaacctacgtgccggcgcaagaaaaaaactttaccaccgctcccgcgatatgtcacgacggaaaggcgcattttccgcgagaaggcgtatttgtatcaaacggaacacactggttcgttacccagcgaaacttctacgagccacagatcattactacggataatacattcgtctctggtaattgcgatgtagtcataggaatagtaaataatacggtttatgaccccctccaacctgagcttgactccttcaaagaggaacttgataaatactttaagaatcatacctctccggatgtcgatctcggggatatttctggtatcaatgcaagcgtcgtgaacatacagaaagaaatcgacagattgaacgaagtcgcgaagaatcttaacgaatctctgatagacttgcaagaattgggcaagtatgagcagggcggccgctacccatacgacgtaccagattacgcttaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 16 - protein, 1336 aa, Artificial Sequence
  Synthetic Polypeptide
MCCTKSLLLAALMSVLLLHLCGESEASNFDCCLGYTDRILHPKFIVGFTRQLANEGCDINAIIFHTKKKLSVCANPKQTWVKYIVRLLSKKVKNMEFNDAQAPKSLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGGGGSGGGGSGGGGSSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSGGGGSGGGGSGGGGSSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGRYPYDVPDYA

SEQ ID NO: 17 - protein, 96 aa, Artificial Sequence
  Synthetic Polypeptide
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 18 - protein, 15 aa, Artificial Sequence
  Synthetic Polypeptide
MFVFLVLLPLVSSQC

SEQ ID NO: 19 - protein, 14 aa, Artificial Sequence
  Synthetic Polypeptide
DSEPVLKGVKLHYT

SEQ ID NO: 20 - protein, 4 aa, Artificial Sequence
  Synthetic Polypeptide
RRAR

SEQ ID NO: 21 - protein, 4 aa, Artificial Sequence
  Synthetic Polypeptide
PSKR

SEQ ID NO: 22 - protein, 5 aa, Artificial Sequence
  Synthetic Polypeptide
GGGGS

SEQ ID NO: 23 - protein, 15 aa, Artificial Sequence
  Synthetic Polypeptide
GGGGSGGGGSGGGGS

SEQ ID NO: 24 - protein, 16 aa, Artificial Sequence
  Synthetic Polypeptide
SGGGGSGGGGSGGGGS

SEQ ID NO: 25 - protein, 4 aa, Artificial Sequence
  Synthetic Polypeptide
GGGS

SEQ ID NO: 26 - protein, 12 aa, Artificial Sequence
  Synthetic Polypeptide
GGGSGGGSGGGS

SEQ ID NO: 27 - protein, 13 aa, Artificial Sequence
  Synthetic Polypeptide
SGGGSGGGSGGGS

SEQ ID NO: 28 - DNA, 81 bp, Artificial Sequence
  Synthetic Polynucleotide
atgtgctgtaccaagagtttgctcctggctgctttgatgtcagtgctgctactccacctctgcggcgaatcagaagcaagc

SEQ ID NO: 29 - protein, 27 aa, Artificial Sequence
  Synthetic Polypeptide
MCCTKSLLLAALMSVLLLHLCGESEAS

SEQ ID NO: 30 - DNA, 27 bp, Artificial Sequence
  Synthetic Polynucleotide
tacccatacgacgtaccagattacgct

SEQ ID NO: 31 - protein, 9 aa, Artificial Sequence
  Synthetic Polypeptide
YPYDVPDYA

SEQ ID NO: 32 - DNA, 24 bp, Artificial Sequence
  Synthetic Polynucleotide
aacgacgctcaggcgccgaagagt

SEQ ID NO: 33 - protein, 8 aa, Artificial Sequence
  Synthetic Polypeptide
NDAQAPKS

SEQ ID NO: 34 - DNA, 12 bp, Artificial Sequence
  Synthetic Polynucleotide
cggcgggcacgt

SEQ ID NO: 35 - DNA, 12 bp, Artificial Sequence
  Synthetic Polynucleotide
ccaagcaagagg

SEQ ID NO: 36 - DNA, 45 bp, Artificial Sequence
  Synthetic Polynucleotide
ggtgggggaggctcaggcggaggtggtagcgggggcggtggttca

SEQ ID NO: 37 - DNA, 45 bp, Artificial Sequence
  Synthetic Polynucleotide
gggggtgggggaagtgggggaggggggagcggcggaggaggtagt

SEQ ID NO: 38 - protein, 15 aa, Artificial Sequence
  Synthetic Polypeptide
GGGGSGGGGSGGGGS

SEQ ID NO: 39 - protein, 15 aa, Artificial Sequence
  Synthetic Polypeptide
GGGGSGGGGSGGGGS

SEQ ID NO: 40 - protein, 65 aa, Artificial Sequence
  Synthetic Polypeptide
YIKWPWYIWLGFIAGLIAIVMVTIMLCCMTSCCSCLKGCCSCGSCCKFDEDDSEPVLKGVKLHYT

SEQ ID NO: 41 - protein, 46 aa, Artificial Sequence
  Synthetic Polypeptide
YIKWPWYIWLGFIAGLIAIVMVTIMLCCMTSCCSCLKGCCSCGSCC

SEQ ID NO: 42 - protein, 19 aa, Artificial Sequence
  Synthetic Polypeptide
KFDEDDSEPVLKGVKLHYT

SEQ ID NO: 43 - protein, 80 aa, Artificial Sequence
  Synthetic Polypeptide
NFDCCLGYTDRILHPKFIVGFTRQLANEGCDINAIIFHTKKKLSVCANPKQTWVKYIVRLLSKKVKNMEFNDAQAPKSLE

SEQ ID NO: 44 - protein, 1273 aa, Artificial Sequence
  Synthetic Polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 45 - protein, 1258 aa, Artificial Sequence
  Synthetic Polypeptide
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 666 aa, Artificial Sequence
  Synthetic Polypeptide
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 47 - protein, 126 aa, Artificial Sequence
  Synthetic Polypeptide
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSK

SEQ ID NO: 48 - protein, 393 aa, Artificial Sequence
  Synthetic Polypeptide
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ